(19) United States
(12) Patent Application Publication
(10) Pub. No.: US 2024/0277828 A1
(43) Pub. Date: Aug. 22, 2024

(54) MULTIVALENT INFLUENZA VACCINES COMPRISING RECOMBINANT HEMAGGLUTININ AND NEURAMINIDASE AND METHODS OF USING THE SAME

(71) Applicant: SANOFI PASTEUR INC, Swiftwater, PA (US)

(72) Inventors: Timothy ALEFANTIS, Spring Brook Township, PA (US); Mario BARRO, Reading, MA (US); Anthony BYERS, Oviedo, FL (US); Guadalupe CORTES-GARCIA, Brookline, MA (US); Philippe-Alexandre GILBERT, Cary, NC (US); Harold KLEANTHOUS, Chelmsford, MA (US); Armaghan NAIK, Natick, MA (US); Konstantin PUGACHEV, Watertown, MA (US); Saranya SRIDHAR, London (GB); Thorsten VOGEL, Melrose, MA (US); William WARREN, Orlando, FL (US)

(21) Appl. No.: 18/653,422

(22) Filed: May 2, 2024

Related U.S. Application Data
(63) Continuation of application No. PCT/US2022/079274, filed on Nov. 4, 2022.
(60) Provisional application No. 63/276,284, filed on Nov. 5, 2021.

(51) Int. Cl. A61K 39/145; A61K 39/00; A61P 37/04

(52) U.S. Cl. CPC A61K 39/145; A61P 37/04; A61K 2039/54; A61K 2039/545; A61K 2039/55555; A61K 2039/55566; A61K 2039/70

(57) ABSTRACT

Disclosed herein are multivalent vaccine or immunogenic compositions comprising one or more recombinant influenza virus hemagglutinin (HA), one or more recombinant influenza virus neuraminidase (NA), and an optional adjuvant. Also disclosed are methods of using the vaccine or immunogenic composition.

### CROSS REFERENCE TO RELATED APPLICATIONS

This application is a continuation application of International Application No. PCT/US2022/079274, filed 4 Nov. 2022, which claims the benefit of, and relies on the filing date of, U.S. provisional patent application No. 63/276,284, filed 5 Nov. 2021, the entire contents of which are incorporated by reference herein.

### SEQUENCE LISTING

This application contains a Sequence Listing which has been submitted electronically in XML format and is hereby incorporated by reference in its entirety. Said XML copy, created on 14 Oct. 2022, is named 0171_0067_PCT_Sequence_Listing.xml and is 3,450 bytes in size.

### FIELD OF THE DISCLOSURE

Disclosed herein are multivalent recombinant influenza vaccine or immunogenic compositions for inducing immunity to both influenza virus hemagglutinin (HA) and influenza virus neuraminidase (NA), and methods of using the multivalent recombinant influenza vaccine or immunogenic compositions.

### BACKGROUND OF THE DISCLOSURE

Influenza is caused by a virus that attacks mainly the upper respiratory tract, including the nose, throat, and bronchi and rarely also the lungs. The infection usually lasts for about a week. It is characterized by sudden onset of high fever, myalgia, headache and severe malaise, non-productive cough, sore throat, and rhinitis. Most people recover within one to two weeks without requiring any medical treatment. However, in the very young, the elderly and people suffering from medical conditions, such as lung diseases, diabetes, cancer, kidney or heart problems, influenza poses a serious risk. In these people, the infection may lead to severe complications of underlying diseases, pneumonia, and death, although even healthy adults and older children can be affected as well. Annual seasonal influenza epidemics are thought to result in between three and five million cases of severe illness and between 250,000 and 500,000 deaths every year around the world.

Influenza virus is a member of the Orthomyxoviridae family. There are three main subtypes of influenza viruses, designated influenza A, influenza B, and influenza C. The influenza virion contains a segmented negative-sense RNA genome, which encodes the following proteins: hemagglutinin (HA), neuraminidase (NA), matrix (M1), proton ion-channel protein (M2), nucleoprotein (NP), polymerase basic protein 1 (PB1), polymerase basic protein 2 (PB2), polymerase acidic protein (PA), and nonstructural protein 2 (NS2). The HA, NA, M1, and M2 are membrane associated, whereas NP, PB1, PB2, PA, and NS2 are nucleocapsid associated proteins. The HA and NA proteins are envelope glycoproteins, primarily responsible for virus attachment and penetration of the viral particles into the cell and release from the cell, respectively.

Certain known licensed influenza vaccine compositions are inactivated vaccines, containing entire virions or virions subjected to treatment with agents that dissolve lipids (“split” vaccines), purified glycoproteins expressed in cell culture (“sub-unit vaccines”), or live attenuated virus vaccines. Other types of vaccines are being developed, such as RNA/DNA based, viral vector based, etc. These vaccines offer protection, in part, by inducing production of antibodies directed against the influenza antigens, such as HA. Antigenic evolution of the influenza virus by mutation, also referred to as antigenic drift, results in modifications in HA and, to a lesser extent, NA. Thus, the amino acid sequences of the major antigens of influenza, including HA and NA, are highly variable across certain groups, subtypes and/or strains.

Accordingly, the available vaccines may only protect against strains having surface glycoproteins that comprise identical or cross-reactive epitopes. To provide a broader antigenic spectrum, conventional vaccines comprise components from several different viral strains, including strains from both Type A and Type B influenza. The choice of strains for use in the current seasonal influenza vaccines is reviewed annually to account for antigenic drift and to match rapidly-evolving viral strains and is predicated on World Health Organization (WHO) recommendations. These recommendations reflect international epidemiological observations.

Current influenza virus seeds for vaccine production must be shown to have the appropriate HA antigen because of the reassortant procedure used to generate high-yielding virus strains used for manufacturing. However, there is currently no requirement for or limit to NA content in influenza vaccines. There is evidence that the NA level in vaccines is quite variable. Kendal et al., Further Studies ofNeuraminidase Content of Inactivated Influenza Vaccines and the Neuraminidase Antibody Responses After Vaccination of Immunologically Primed and Unprimed Populations, INFECTION AND IMMUNITY 1980; 29(3):966-971, reported that the NA specific activity for different lots may range approximately 40-fold. Kendal et al. also noted a rapid decline of NA activity during six months of storage. As a result, the frequency of antibody response to NA was poor (mean seroconversion rate of 18%) compared to the HA response (seroconversion rate of 64%).

Moreover, despite growing evidence showing that NA-specific antibodies correlate with resistance to disease in humans, current vaccination strategies focus almost entirely on the HA antigen or entirely on the HA antigen, as in the case of the FLUBLOK® quadrivalent vaccine comprising recombinant HA proteins. In addition, there is limited data available regarding the immunological response to NA during influenza infection, particularly as compared to the data for HA (Wong et al., Hemagglutinin and Neuraminidase Antibodies Are Induced in Age- and Subtype-Dependent Manner after Influenza Virus Infection, JOURNAL OF VIROLOGY 2020; 94(7):e01385-19). The influenza virus naturally contains about ten times less NA on the viral surface compared to HA, and the established processes to enrich the HA antigen may not be amenable to maintaining NA in its enzymatically active and tetrameric conformation. Thus, while the currently available inactivated influenza virus vaccines may contain NA, the quantity and quality vary widely and are not uniform.

Furthermore, NA has been described to be immunosubdominant when presented to the immune system together with HA (Krammer, The human antibody response to influenza A virus infection and vaccination, NATURE REVIEWS IMMUNOLOGY 2019; 19:383-397). Put another way, HA is known to be immunodominant over NA. Id. This phenomenon of immunodominance, observed in conventional influenza vaccines, remains an obstacle to the development of multivalent vaccines that can successfully achieve polyvalent immune responses against multiple antigens or epitopes, particularly for multivalent vaccines that contain an immunodominant protein, such as HA, and/or as the number of valencies in the vaccine increases (Woodruff et al., B Cell Competition for Restricted T Cell Help Suppresses Rare-Epitope Responses, CELL REPORTS 2018; 25:321-27).

Thus, the ability to supplement influenza virus HA in vaccines with one or more influenza virus NA proteins, which may confer enhanced protection and/or broader breadth of protection against circulating influenza strains by inducing both an HA and a NA immune response, is desirable. However, the ability to combine influenza virus HA and influenza virus NA into a vaccine composition that confers enhanced protection and/or broader breadth of protection against circulating influenza strains, without antigenic competition, can present a challenge, particularly in a multivalent vaccine composition.

### SUMMARY OF THE DISCLOSURE

The present disclosure provides a vaccine or immunogenic composition comprising a plurality of recombinant influenza virus proteins, wherein the plurality of recombinant influenza virus proteins comprises or consists of one or more recombinant influenza virus HA and one or more recombinant influenza virus NA.

In various embodiments, the plurality of recombinant influenza virus proteins comprises one, two, three, four, five, six, seven, eight, or more recombinant influenza virus HA antigens and one, two, three, four, five, six, seven, eight, or more recombinant influenza virus NA antigens. In certain embodiments, the plurality of recombinant influenza virus proteins comprises or consists of four influenza virus HA and four influenza virus NA. In certain embodiments, the plurality of recombinant influenza virus proteins comprises or consists of a first recombinant influenza virus HA wherein the first recombinant influenza virus HA is an H1 HA; a second recombinant influenza virus HA wherein the second recombinant influenza virus HA is an H3 HA; a third recombinant influenza virus HA wherein the third recombinant influenza virus HA is from a B/Victoria lineage; a fourth recombinant influenza virus HA wherein the fourth recombinant influenza virus HA is from a B/Yamagata lineage; a first recombinant influenza virus NA wherein the first recombinant influenza virus NA is an N1 NA; a second recombinant influenza virus NA wherein the second recombinant influenza virus NA is an N2 NA; a third recombinant influenza virus NA wherein the third recombinant influenza virus NA is from a B/Victoria lineage; and a fourth recombinant influenza virus NA wherein the fourth recombinant influenza virus NA is from a B/Yamagata lineage.

In various embodiments of the vaccine or immunogenic composition disclosed herein, each of the first, second, third, and fourth recombinant influenza virus NA is a modified recombinant influenza virus NA. In certain embodiments, the modified recombinant influenza virus NA comprises a modified recombinant tetrameric influenza virus NA comprising four modified recombinant monomeric NA molecules, each comprising a head region of the NA of the influenza virus, but lacking a cytoplasmic tail, a transmembrane region, and all or substantially all of a stalk region of the NA of the influenza virus and wherein the modified monomeric NA molecules form modified recombinant tetrameric NA when expressed in a host cell. In certain embodiments, each modified recombinant monomeric influenza virus NA comprises a heterologous tetramerization domain, and in certain embodiments, the modified recombinant monomeric influenza virus NA does not comprise a heterologous oligomerization domain.

In certain embodiments of the disclosure, the heterologous tetramerization domain is a Staphylothermus marinus tetrabrachion tetramerization domain, a GCN4 leucine zipper tetramerization domain, a tetramerization domain from a paramyxovirus phosphoprotein, or a human vasodilator stimulated phosphoprotein (VASP) tetramerization domain.

In one aspect of the disclosure, each of the recombinant influenza virus HA is produced by a baculovirus expression system, for example a baculovirus expression system in cultured insect cells. In one aspect of the disclosure, each of the recombinant influenza virus NA is produced in Chinese Hamster Ovary (CHO) cells.

In various embodiments disclosed herein, the vaccine or immunogenic composition does not contain inactivated influenza virions or live attenuated influenza virions, and in various embodiments, each of the recombinant influenza virus HAs and/or each of the recombinant influenza virus NAs are from standard of care influenza strains. In certain embodiments, the H1 HA is from an H1N1 influenza virus strain and/or the H3 HA is from an H3N2 influenza virus strain, and in certain embodiments, the N1 NA is from an H1N1 influenza virus strain and/or the N2 NA is from an H3N2 influenza virus strain. In certain embodiments, the H1 HA is from an H1N1 influenza virus strain, the H3 HA is from an H3N2 influenza virus strain, the N1 NA is from an H1N1 influenza virus strain, and the N2 NA is from an H3N2 influenza virus strain. In certain embodiments, the H1 HA and the N1 NA are from the same H1N1 influenza virus strain and the H3 HA and N2 NA are from the same H3N2 influenza virus strain.

In certain embodiments, the vaccine or immunogenic composition disclosed herein further comprises an adjuvant, and in certain embodiments, the adjuvant comprises a squalene-in-water adjuvant, such as AF03, or a liposome-based adjuvant, such as SPA14.

In certain aspects of the disclosure, each of the recombinant influenza virus HAs is present in the vaccine or immunogenic composition in an amount ranging from about 0.1 μg to about 90 μg, optionally from about 1 μg to about 60 μg or from about 5 μg to about 45 μg, and in certain aspects, each of the recombinant influenza virus NAs is present in the vaccine or immunogenic composition in an amount ranging from about 0.1 μg to about 90 μg, optionally from about 1 μg to about 60 μg or from about 5 μg to about 45 μg. In certain embodiments, the composition is formulated for intramuscular injection.

In another aspect, disclosed herein is a vaccine comprising the immunogenic composition disclosed herein and a pharmaceutical carrier.

Also disclosed herein are methods of immunizing a subject against influenza virus comprising administering to the subject an immunologically effective amount of the vaccine as disclosed herein. Also disclosed herein is a vaccine as disclosed herein for use in a method of immunizing a subject against influenza virus. Also disclosed herein is an immunogenic composition as disclosed herein for the manufacture of a vaccine for use in a method of immunizing a subject against influenza virus. In certain embodiments, the method or use prevents influenza virus infection in the subject, and in certain embodiments, the method or use raises a protective immune response, such as an HA antibody response and/or an NA antibody response, in the subject. In certain embodiments, the subject is human, and in certain embodiments, the vaccine is administered or is prepared to be administered intramuscularly, intradermally, subcutaneously, intravenously, intranasally, by inhalation, or intraperitoneally.

Another aspect of the disclosure is directed to a method of reducing one or more symptoms of influenza virus infection, the method comprising administering to a subject a prophylactically effective amount of the vaccine disclosed herein. Also disclosed herein is a vaccine as disclosed herein for use in a method of reducing one or more symptoms of influenza virus infection. Also disclosed herein is an immunogenic composition as disclosed herein for the manufacture of a vaccine for use in a method of reducing one or more symptoms of influenza virus infection

Also disclosed herein is a method of enhancing or broadening a protective immune response in a subject, the method comprising administering to the subject an immunologically effective amount of the vaccine disclosed herein, wherein the vaccine increases the vaccine efficacy of a standard of care influenza virus vaccine composition by an amount ranging from about 5% to about 100%, such as from about 10% to about 25%, or from about 40% to about 80%, or from about 40% to about 60%. Also disclosed herein is a vaccine as disclosed herein for use in a method of enhancing or broadening a protective immune response in a subject, the method comprising administering to the subject an immunologically effective amount of the vaccine disclosed herein, wherein the vaccine increases the vaccine efficacy of a standard of care influenza virus vaccine composition by an amount ranging from about 5% to about 100%, such as at least about 20%, or from about 40% to about 80%, such as from about 40% to about 60%. Also disclosed herein is an immunogenic composition as disclosed herein for the manufacture of a vaccine for use in a method of enhancing or broadening a protective immune response in a subject, the method comprising administering to the subject an immunologically effective amount of the vaccine disclosed herein, wherein the vaccine increases the vaccine efficacy of a standard of care influenza virus vaccine composition by an amount ranging from about 5% to about 100%, such as at least about 20%, or from about 40% to about 80%, such as from about 40% to about 60%. In certain embodiments, the standard of care influenza virus vaccine is an inactivated influenza virus composition comprising inactivated influenza virus from an H1N1 strain, an H3N2 strain, a B/Victoria lineage, and a B/Yamagata lineage. In certain embodiments, the standard of care influenza virus vaccine composition comprises recombinant influenza virus HA from an H1N1 strain, an H3N2 strain, a B/Victoria lineage, and a B/Yamagata lineage.

In various embodiments, the methods or uses and compositions disclosed herein treat or prevent disease caused by either or both a seasonal and a pandemic influenza strain. In certain embodiments of the methods or uses disclosed herein wherein the subject is human, the human is 6 months of age or older, less than 18 years of age, at least 6 months of age and less than 18 years of age, at least 18 years of age and less than 65 years of age, at least 6 months of age and less than 5 years of age, at least 5 years of age and less than 65 years of age, at least 60 years of age, or at least 65 years of age. In certain embodiments, the methods or uses disclosed herein comprise administering to the subject two doses of the vaccine or immunogenic composition with an interval of 2-6 weeks, such as an interval of 4 weeks.

### BRIEF DESCRIPTION OF THE FIGURES

FIG. 1 is a schematic representation and partial amino acid sequence of rTET-NA (SEQ ID NO: 2). SEQ ID NO: 2 represents in order a CD5 signal sequence, a first linker sequence, a tetrabrachion tetramerization domain, and a second linker sequence. SEQ ID NO: 2 does not include the amino acid sequence of the NA head region.

FIG. 2A is a schematic illustrating the experimental design of vaccination in mice, as discussed in Example 2.

FIG. 2B is a plot showing the IC₅₀ of NA inhibition against A/Singapore/INFIMH-16-0019/2016 (N2) for mice vaccinated using rTET-NA, live virus-derived NA (LVNA), or monovalent inactivated influenza vaccine (IIV), both with and without adjuvant (AF03), as described in Example 2. The ∘ symbol represents IC₅₀ NA inhibition titers without AF03 addition and ▪ represents groups with AF03 addition.

FIG. 2C is a plot showing the IC₅₀ of NA inhibition against A/Michigan/45/2015 (N1) for vaccination of mice using rTET-NA or monovalent inactivated influenza vaccine (IIV), both with and without adjuvant (AF03), as described in Example 2. The ∘ symbol represents IC₅₀ NA inhibition titers without AF03 addition and ▪ represents groups with AF03 addition.

FIG. 3A is a schematic illustrating the experimental design of vaccination in naïve ferrets receiving two intramuscular doses of the vaccine samples on days 0 and 21, with final bleed on day 42, as described in Example 3.

FIG. 3B is a schematic illustrating the experimental design of vaccination in pre-immune ferrets (virus-primed intranasally on day 0) as discussed in Example 3.

FIG. 3C is a plot showing NAI titers against A/Singapore/Infimh/16/2016 (N2) in naïve ferrets after 1 or 2 immunizations with the following dosages of rTET-NA: diluent only (mock), 5 μg+AF03; 45 μg+AF03, and 45 μg, as described in Example 3. The ∘ symbol represents NAI titers after a first dose and ▪ represents NAI titers after a second dose.

FIG. 3D is a plot showing NAI titers against A/Singapore/Infimh/16/2016 (N2) in pre-immune ferrets after a single dose of diluent (mock), rTET-NA 1.8 μg, 9 μg, and 45 μg, and IIV 1.8 μg and 9 μg, as described in Example 3. The ∘ symbol represents NAI titers after intranasal virus prime and ▪ represents NAI titers after a single intramuscular vaccine boost.

FIG. 3E is a graph showing the NAI ratio of boost/prime against A/Singapore/Infimh/16/2016 (N2) in pre-immune ferrets after a single dose of diluent (mock), rTET-NA 1.8 μg, 9 μg, and 45 μg, and IIV 1.8 μg and 9 μg, as described in Example 3.

FIG. 3F is a plot showing NAI titers against A/Michigan/45/2015 (N1) in naïve ferrets after 1 or 2 immunizations with the following dosages of rTET-NA: diluent (mock), 5 μg+AF03; 45 μg+AF03, and 45 μg, as described in Example 3. The ∘ symbol represents NAI titers after a first dose and ▪ represents NAI titers after a second dose.

FIG. 3G is a plot showing NAI titers against A/Michigan/45/2015 (N1) in pre-immune ferrets after a single dose of diluent (mock), rTET-NA 1.8 μg, 9 μg, and 45 μg, and IIV 1.8 μg and 9 μg, as described in Example 3. The ∘ symbol represents NAI titers after intranasal virus prime and ▪ represents NAI titers after a single intramuscular vaccine boost.

FIG. 3H is a graph showing the NAI ratio of boost/prime against A/Michigan/45/2015 (N1) in pre-immune ferrets after a single dose of diluent (mock), rTET-NA 0.36 μg, 1.8 μg, 9 μg, and 45 μg, and IIV 1.8 μg and 9 μg, as described in Example 3.

FIG. 4 is a plot showing NAI titers against A/Perth/16/2009 (N2) in naïve ferrets after 2 immunizations with the following dosages of rTET-NA: diluent+AF03 (mock), 0.2 μg, 3 μg, 45 μg, 0.2 μg+AF03, 3 μg+AF03, and 45 μg+AF03, as described in Example 4. NAI titers after infection with A/Perth/16/2009 H3N2 influenza virus are also shown (A/PE/09 pre-infected).

FIG. 5 are graphs showing post-challenge body weight change (daily and AUC), temperature rise (peak), and virus shedding (AUC) in ferrets previously immunized with the following dosages of rTET-NA: diluent+AF03 (mock), 0.2 μg, 3 μg, 45 μg, 0.2 μg+AF-03, 3 μg+AF03, 45 μg+AF03, as well as after infection with A/Perth/16/2009 H3N2 influenza virus (A/PE/09 pre-infected), as described in Example 4.

FIG. 6A is a graph showing an inverse correlation between disease severity and NAI titers in vaccinated ferrets, as described in Example 4, wherein NAI titers in ferrets with non-severe disease are shown on the left, and NAI titers in ferrets with severe disease are shown on the right.

FIG. 6B is a graph showing a receiver operating characteristics (ROC) curve model illustrating the area under the curve (AUC) of the ROC curve, wherein the AUC is significantly higher than chance, as discussed in Example 4.

FIG. 6C is a graph showing the inverse correlation between disease severity and NAI titers in vaccinated ferrets on Day 42, as described in Example 4.

FIG. 7A is a schematic illustrating the experimental design of vaccination in ferrets, as discussed in Example 5.

FIG. 7B is a chart showing the influenza virus strains used in the 4× rNA and 4× rHA vaccine strain selection in ferrets as discussed in Example 5.

FIG. 7C (left column) are plots showing NAI titers against A/Singapore/Infimh/16/2016 (N2) (top row); A/Michigan/45/2015 (N1) (second row); B/Colorado/06/2017 (third row); and B/Phuket/3073/2013 (bottom row) after vaccination with (1) one dose of 45 μg/antigen or 5 μg/antigen+adjuvant of an octavalent (4× rHA+4× rNA) recombinant vaccine composition, (2) one dose of a quadrivalent (4× rNA) recombinant vaccine composition, or (3) one dose of a quadrivalent (4× rHA) recombinant vaccine composition, as described in Example 5.

FIG. 7C (right column) is a plot showing NAI titers against A/Singapore/Infimh/16/2016 (N2) (top row); A/Michigan/45/2015 (N1) (second row); B/Colorado/06/2017 (third row); and B/Phuket/3073/2013 (bottom row) after vaccination with (1) a booster dose of 45 μg/antigen or 5 μg/antigen+adjuvant of an octavalent (4×rHA+4×rNA) recombinant vaccine composition, (2) a booster dose of a quadrivalent (4×rNA) recombinant vaccine composition, or (3) a booster dose of a quadrivalent (4×rHA) recombinant vaccine, as described in Example 5. The open squares represent NAI titers after receiving the octavalent recombinant composition, closed squares represent NAI titers after receiving the quadrivalent (4×rNA) recombinant composition, and triangles represent NAI titers after receiving the quadrivalent (4×rHA) recombinant composition.

FIG. 8A is a plot showing HAI titers against A/Singapore/Infimh/16/2016 H3N2 virus after vaccination with either 45 μg/antigen or 5 μg/antigen+adjuvant of (1) quadrivalent rNA (closed squares); (2) quadrivalent rHA (triangles); or (3) octavalent rHA+rNA (open squares), as described in Example 5.

FIG. 8B is a plot showing IgG titers measured by Antibody Forensics against H3 rHA bead panel after vaccination with 45 μg/antigen of (1) octavalent rHA+rNA (Y axis) or (2) quadrivalent rHA (X axis), as described in Example 6.

FIG. 8C is a plot showing IgG titers measured by Antibody Forensics against H3 rHA bead panel after vaccination with 5 μg/antigen+adjuvant of (1) octavalent rHA+rNA (Y axis) or (2) quadrivalent rHA (X axis), as described in Example 6.

FIG. 8D is a plot showing HAI titers against A/Michigan/45/2015 H1N1 virus after vaccination with either 45 μg/antigen or 5 μg/antigen+adjuvant of (1) quadrivalent rNA (closed black squares); (2) quadrivalent rHA (triangles); or (3) octavalent rHA+rNA (open squares), as described in Example 5.

FIG. 8E is a plot showing IgG titers measured by Antibody Forensics against H1 rHA bead panel after vaccination with 45 μg/antigen of (1) octavalent rHA+rNA (Y axis) or (2) quadrivalent rHA (X axis), as described in Example 6.

FIG. 8F is a plot showing IgG titers measured by Antibody Forensics against H1 rHA bead panel after vaccination with 5 μg/antigen+adjuvant of (1) octavalent rHA+rNA (Y axis) or (2) quadrivalent rHA (X axis), as described in Example 6.

### DETAILED DESCRIPTION OF THE DISCLOSURE

Some viruses are capable of substantial variation in the structure of their envelope glycoprotein components. Influenza virus, for example, constantly changes the amino acid sequence of its envelope glycoproteins. Either major amino acid variations (antigenic shift) or minor variations (antigenic drift) can give rise to new epitopes, allowing the virus to evade the immune system. The antigenic variation is the major cause of repeated influenza outbreaks. Antigenic variants within a subtype (e.g., H1 or H3) emerge and are gradually selected as predominant virus while the preceding virus is suppressed by specific antibody arising in the population. Neutralizing antibody to one variant generally becomes less and less effective as sequential variants arise. The immune response to variants within a subtype may depend on the prior experience of the host.

HA and NA evolve quite differently. For example, the rate of silent nucleotide substitution has been shown to be higher than the rate of coding nucleotide substitutions for all genes of influenza virus, including the gene for HA (Webster, R. G., et al., Evolution and ecology of influenza A viruses, MICROBIOL. REVS. 1992; 56(1):152-179). However, HA has a much higher rate of coding changes than the internal proteins. The elevated rate of coding nucleotide changes in the HA gene as compared with other genes has been taken as evidence that immune selection is an important factor in its evolution (Palese, P., et al., Variation of Influenza A, B, and C Viruses, SCIENCE 1982; 215(4539):1468-74). Using reassorted antigens to eliminate any nonspecific steric hindrance, Kilbourne et al. studied the rate of evolution of epidemiologically important HA and NA antigens isolated from humans over a 10-year period and determined that the HA evolved more rapidly than the NA (Kilbourne, E. D., et al., Independent and disparate evolution in nature of influenza virus A hemagglutinin and neuraminidase glycoproteins, PNAS 1990; 87(2):786-790). This was shown with both Type A H1N1 and H3N2 viruses and has been confirmed by subsequent experiments with more recent strains. The reason for the apparently different rates of evolution is unknown but may be due to the fact that antibody to HA neutralizes virus and prevents infection. This places more selective pressure on the HA to maintain itself in a partially immune population. Thus, because NA undergoes more gradual antigenic drift as compared with HA, a vaccine or immunogenic composition comprising both HA and NA may offer a broader protection (in the form of NA antibodies) against strains of influenza containing antigenically-drifted HA antigen.

Because the influenza virus naturally contains about ten times less NA on the viral surface compared to HA and because the established process to enrich the HA antigen may not be amenable to maintaining NA in its enzymatically active and tetrameric conformation, the amount of NA detectable in vaccines compositions, such as inactivated viral vaccines, may by quite variable. Therefore, the addition of recombinant NA to a vaccine or immunogenic composition as disclosed herein may allow for better control over the amount of NA contained in a vaccine or immunogenic composition. Producing stable NA recombinantly and adding it to HA antigen, such as recombinantly-produced HA antigen, may allow for better balancing of both the HA and NA immune responses in subjects receiving the vaccine or immunogenic composition, and, in turn, enhanced protection and/or broader breadth of protection against circulating influenza strains, as compared to currently available vaccines.

Accordingly, disclosed herein are multivalent vaccine or immunogenic compositions comprising a plurality of recombinant influenza virus proteins, including a plurality of recombinant influenza virus HA and a plurality of recombinant influenza virus NA.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Adjuvant: As used herein, the term “adjuvant” refers to a substance or combination of substances that may be used to enhance an immune response to an antigen component of a vaccine.

Antigen: As used herein, the term “antigen” refers to an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, ferrets, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity, including those induced by heterologous immunogens. Antigens include the NA and HA forms as described herein.

Approximately: As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Epitope: As used herein, the term “epitope” includes any moiety that is specifically recognized by an immunoglobulin (e.g., antibody or T-cell receptor) binding component in whole or in part. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms or groups are physically separated from one another when the antigen adopts an alternative conformation (e.g., is linearized).

Excipient: As used herein, the term “excipient” refers to a non-therapeutic agent that may be included in a pharmaceutical composition, for example to provide or contribute to a desired consistency or stabilizing effect. Suitable pharmaceutical excipients include, for example, starch, glucose, lactose, sucrose, sorbitol, gelatin, malt, rice, flour, chalk, silica gel, sodium stearate, glycerol monostearate, talc, sodium chloride, dried skim milk, glycerol, propylene, glycol, water, ethanol and the like.

H1: As used herein, “1-” refers to an influenza virus subtype 1 hemagglutinin (HA). Type A influenza viruses are divided into Groups 1 and 2. Groups 1 and 2 are further divided into subtypes, which refers to classification of a virus based on the sequences of two proteins on the surface of the virus HA and neuraminidase (NA). Currently, there are 18 recognized HA subtypes (H1-H18). H1 is thus distinct from the other HA subtypes, including 1H2-H18.

H3: As used herein, “H3” refers to an influenza virus subtype 3 HA. H3 is thus distinct from the other H A subtypes, including H1, H2 and H4-H18.

Immune response: As used herein, the term “immune response” refers to a response of a cell of the immune system, such as a B cell, T cell, dendritic cell, macrophage or polymorphonucleocyte, to a stimulus such as an antigen, immunogen, or vaccine. An immune response can include any cell of the body involved in a host defense response, including for example, an epithelial cell that secretes an interferon or a cytokine. An immune response includes, but is not limited to, an innate and/or adaptive immune response. Methods of measuring immune responses are well known in the art and include, for example, measuring proliferation and/or activity of lymphocytes (such as B or T cells), secretion of cytokines or chemokines, inflammation, antibody production and the like. An antibody response or humoral response is an immune response in which antibodies are produced. A “cellular immune response” is one mediated by T cells and/or other white blood cells.

Immunogen: As used herein, the term “immunogen” or “immunogenic” refers to a compound, composition, or substance which is capable, under appropriate conditions, of stimulating an immune response, such as the production of antibodies or a T cell response in an animal, including compositions that are injected or absorbed into an animal. As used herein, the term “immunogenic composition” refers to a composition that generates an immune response that may or may not be a protective immune response. As used herein, “immunize” means to induce in a subject a protective immune response against an infectious disease (e.g., influenza).

Immunologically effective amount: As used herein, the term “immunologically effective amount” means an amount sufficient to immunize a subject.

In some embodiments: As used herein, the term “in some embodiments” refers to embodiments of all aspects of the disclosure, unless the context clearly indicates otherwise.

Machine learning: As used herein, the term “machine learning” refers to the use of algorithms that improve automatically through experience and/or by the use of data. Machine learning may involve construction of a predictive model, such as a model of influenza antigenicity, to allow prediction of data, including the use of an algorithm designed to select candidate antigens through the predictive model. Target strains may be identified and a selection algorithm may then be constructed. Examples of machine learning algorithms and methods can be found, for example, in PCT Application Nos. WO 2021/080990 A1, entitled Systems and Methods for Designing Vaccines, and WO 2021/080999 A1, entitled Systems and Methods for Predicting Biological Responses, both of which are incorporated by reference in their entireties herein. Machine learning, as used herein, may also include the application of computation tools to analyze and interpret data, for example, bioinformatics analyses, such as phylogenetic analysis. Likewise, a “machine learning influenza virus HA” indicates an influenza virus HA that has been identified or designed by machine learning, and a “machine learning influenza virus NA” indicates an influenza virus NA that has been identified or designed by machine learning. A “machine learning model” indicates a model that uses algorithms that improve automatically through experience and/or by the use of data in order to predict data, such as a candidate antigen.

Modified: As used herein, the term “modified” refers to any protein or nucleic acid that has a different amino acid or nucleic acid sequence as compared to a wild-type form of the protein or nucleic acid. For example, a modified influenza NA or HA refers to an influenza NA or HA that has an amino acid or nucleic acid sequence that differs from a wild type NA protein or nucleic acid sequence. The modified influenza NA or HA may comprise one or more amino acid deletions and/or substitutions relative to a wild type influenza NA or HA.

Monomeric influenza virus neuraminidase: Wild-type influenza virus neuraminidase (NA) is a tetramer of four identical monomers. Each NA monomer in the wild-type influenza NA consists of four distinct structural domains: the enzymatic head region, the stalk region, the transmembrane region, and the cytoplasmic tail. As used herein, the term “monomeric influenza virus neuraminidase” refers to a NA monomer that can combine with three other NA monomers to form tetrameric NA. As described herein, a modified monomeric influenza virus neuraminidase may include a head region of an influenza virus NA but include a heterologous tetramerization domain or fraction thereof and/or lack at least a portion of one or more of the cytoplasmic tail, the transmembrane region, and the stalk region.

N1: As used herein, “N1” refers to an influenza virus subtype 1 neuraminidase (NA). Type A influenza viruses are divided into Groups 1 and 2. Groups 1 and 2 are further divided into subtypes, which refers to classification of a virus based on the sequences of two proteins on the surface of the virus HA and neuraninidase (NA). Currently, there are I1 recognized NA subtypes (N1-N11). N1 is thus distinct from the other NA subtypes, including N2-N11. N2: As used herein, “N2” refers to an influenza virus subtype 2 neuraminidase (NA). N2 is thus distinct from the other NA subtypes, including N1 and N3-N11.

Influenza B strains are classified into two lineages: B/Yamagata and B/Victoria.

Pandemic strain: A “pandemic” influenza strain is one that has caused or has capacity to cause pandemic infection of subject populations, such as human populations. In some embodiments, a pandemic strain has caused pandemic infection. In some embodiments, such pandemic infection involves epidemic infection across multiple territories; in some embodiments, pandemic infection involves infection across territories that are separated from one another (e.g., by mountains, bodies of water, as part of distinct continents, etc.) such that infections ordinarily do not pass between them.

Prevention: The term “prevention”, as used herein, refers to prophylaxis, avoidance of disease manifestation, a delay of onset, and/or reduction in frequency and/or severity of one or more symptoms of a particular disease, disorder or condition (e.g., infection for example with influenza virus). In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

Recombinant: As used herein, the term “recombinant” is intended to refer to polypeptides (e.g., HA and/or NA polypeptides as described herein) that are designed, engineered, prepared, expressed, created or isolated by recombinant means, such as polypeptides expressed using a recombinant expression vector transfected into a host cell, polypeptides isolated from a recombinant, combinatorial polypeptide library or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more of such selected sequence elements results from mutagenesis (e.g., in vivo or in vitro) of a known sequence element, e.g., from a natural or synthetic source. In some embodiments, one or more of such selected sequence elements results from the combination of multiple (e.g., two or more) known sequence elements that are not naturally present in the same polypeptide (e.g., two epitopes from two separate HA polypeptides or two separate NA polypeptides). Recombinant HA is rHA, and recombinant NA is rNA.

Seasonal strain: A “seasonal” influenza strain is one that has caused or has capacity to cause a seasonal infection (e.g., annual epidemic) of subject populations, such as human populations. In some embodiments, a seasonal strain has caused seasonal infection.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. “Sequence identity” between two nucleic acid sequences indicates the percentage of nucleotides that are identical between the sequences. “Sequence identity” between two amino acid sequences indicates the percentage of amino acids that are identical between the sequences. Homologs or variants of a given gene or protein will possess a relatively high degree of sequence identity when aligned using standard methods.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90%, at least about 95%, at least about 96%, at least about 97%, at least about 98%, at least about 99%, or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional and/or structural property of said given sequence, e.g., and in some instances, are functionally and/or structurally equivalent to said given sequence. In some embodiments, a nucleic acid sequence or amino acid sequence having a particular degree of identity to a given nucleic acid sequence or amino acid sequence is functionally and/or structurally equivalent to said given sequence.

Standard of Care Strain: Each year, based on intensive surveillance efforts, the World Health Organization (WHO) selects influenza strains to be included in the seasonal vaccine preparations. As used herein, the term “standard of care strain” or “SOC strain” refers to an influenza strain that is selected by the World Health Organization (WHO) to be included in the seasonal vaccine preparations. A standard of care strain can include a historical standard of care strain, a current standard of care strain or a future standard of care strain.

Subject: As used herein, the term “subject” means any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In some embodiments, the non-human subject is a mammal (e.g., a rodent, a mouse, a rat, a rabbit, a ferret, a monkey, a dog, a cat, a sheep, cattle, a primate, and/or a pig). In some embodiments, a subject may be a transgenic animal, genetically-engineered animal, and/or a clone. In some embodiments, the subject is an adult, an adolescent or an infant. In some embodiments, terms “individual” or “patient” are used and are intended to be interchangeable with “subject.”

Tetrameric NA molecule: As used herein, the term “tetrameric NA molecule” refers to a compound that includes four NA monomeric polypeptide units. In some embodiments, each monomeric NA molecule in a given tetrameric NA compound includes a globular head domain, a stalk region, a hydrophobic transmembrane domain, and a short, N-terminal cytoplasmic domain. In some embodiments, one or more of these domains or regions of a given monomeric NA molecule are truncated, altogether absent, or modified relative to a reference wild-type monomeric NA molecule.

Tetramerization domain: As used herein, the term “tetramerization domain” refers to an amino acid sequence encoding a domain that causes the tetrameric assembly of a polypeptide or protein. A tetramerization domain that is not native to a particular protein may be termed an artificial or a heterologous tetramerization domain. Exemplary tetramerization domains include, but are not limited to, sequences from Tetrabrachion, GCN4 leucine zippers, or vasodilator-stimulated phosphoprotein (VASP).

Vaccine composition: As used herein, the term “vaccine composition” or “vaccine” refers to a composition that generates a protective immune response in a subject. As used herein, a “protective immune response” refers to an immune response that protects a subject from infection (prevents infection or prevents the development of disease associated with infection) or reduces the symptoms of infection (for instance an infection by an influenza virus). Vaccines may elicit both prophylactic (preventative) and therapeutic responses. Methods of administration vary according to the vaccine, but may include inoculation, ingestion, inhalation or other forms of administration. Inoculations can be delivered by any of a number of routes, including parenteral, such as intravenous, subcutaneous, intraperitoneal, intradermal, or intramuscular. Vaccines may be administered with an adjuvant to boost the immune response.

Vaccinate: As used herein, the term “vaccinate” or the like refers to the administration of a vaccine composition to generate a protective immune response in a subject, for example to a disease-causing agent such as an influenza virus. Vaccination can occur before, during, and/or after exposure to a disease-causing agent, and/or to the development of one or more symptoms, and in some embodiments, before, during, and/or shortly after exposure to the agent. In some embodiments, vaccination includes multiple administrations, appropriately spaced in time, of a vaccine composition.

Vaccine Efficacy: As used herein, the term “vaccine efficacy” or “vaccine effectiveness” refers to a measurement in terms of percentage of reduction in evidence of disease among subjects who have been administered a vaccine. For example, a vaccine efficacy of 50% indicates a 50% decrease in the number of disease cases among a group of vaccinated subjects as compared to a group of unvaccinated subjects or a group of subjects administered a different vaccine.

Wild type (WT): As is understood in the art, the term “wild type” generally refers to a normal form of a protein or nucleic acid, as is found in nature. For example, wild type HA and NA polypeptides are found in natural isolates of influenza virus. A variety of different wild type HA and NA sequences can be found in the NCBI influenza virus sequence database.

### Nomenclature for Influenza Virus

All nomenclature used to classify influenza virus is that commonly used by those skilled in the art. Thus, a Type, or Group, of influenza virus refers to the three main types of influenza: influenza Type A, influenza Type B or influenza Type C that infect humans. Influenza A and B cause significant morbidity and mortality each year. It is understood by those skilled in the art that the designation of a virus as a specific Type relates to sequence difference in the respective Ml (matrix) protein or P (nucleoprotein). Type A influenza viruses are further divided into group 1 and group 2. These groups are further divided into subtypes, which refers to classification of a virus based on the sequences of two proteins on the surface of the virus HA and NA. Currently, there are 18 recognized HA subtypes (H1-H18) and 11 recognized NA subtypes (N1-N11). Group 1 contains N1, N4, N5, and N8 and H1, H2, H5, H6, H8, H9, H11, H12, H13, H16, H17 and H18. Group 2 contains N2, N3, N6, N7, and N9 and H3, H4, H7, H10, H14, and H15. N10 and N11 have been identified in influenza-like genomes isolated from bats (Wu et al., Bat-derived influenza-like viruses 117N1O and H18N11, TRENDS IN MICROBIOLOGY, 2014, 22(4):183-91). While there are potentially 198 different influenza A subtype combinations, only about 131 subtypes have been detected in nature. Current subtypes of influenza A viruses that commonly circulate in the human population, giving rise to seasonal outbreaks, include A(H1N1) and A(H3N2).

Influenza A subtypes can be further broken down into different genetic “clades” and “sub-clades.” For example, A subtype A(H1N1) contains clade 6B.1 and sub-clade 6B.1A. A subtype A(H3N2) contains clades 3C.2A and 3C.3A and sub-clades 3C.2A1, 3C.2A2, 3C2A3, and 3C.2A4. Likewise, B subtype Victoria contains clade VIA and sub-clades V1A.1, V1A.2, and V1A.3, while B subtype Yamagata contains clades Y1, Y2, and Y3. Finally, the term strain refers to viruses within a subtype that differ from one another in that they have small, genetic variations in their genome.

For convenience, certain abbreviations can be used to refer to protein constructs, and portions thereof, described herein. For example, HA can refer to an influenza hemagglutinin protein. H1 refers to HA from an influenza subtype 1 strain. H3 refers to HA from an influenza subtype 3 strain. Likewise, NA can refer to influenza neuraminidase protein, or a portion thereof. N2 refers to neuraminidase from an influenza subtype 2 strain. The term tet-NA or rTET-NA refers to a recombinant NA comprising a heterologous tetramerization domain that forms tetrameric NA when expressed in cells, HA refers to hemagglutinin or a portion thereof.

### Hemagglutinin (HA)

Hemagglutinin (HA), along with NA, is one of the two major influenza surface proteins. The functions of both NA and HA involve interactions with sialic acid, a terminal molecule bound to sugar moieties on glycoproteins or glycolipids expressed on the surface of cells. The binding of HA to sialic acid on the cell surface induces endocytosis of the virus by the cell, allowing the virus to gain entry and infect cells. Sialic acid is also added to HA and NA as part of the glycosylation process that occurs within infected cells.

HA is believed to mediate attachment of the influenza virus to the host cell and viral-cell membrane fusion during penetration of the virus into the cell. Antigenic variation in the HA molecule is responsible for frequent outbreaks to influenza and for limited control of infection by immunization.

HA is present in mature influenza virus as trimers. Each HA monomer consists of two polypeptides (HA1 and HA2) linked by a disulfide bond. These polypeptides are derived by cleavage of a single precursor protein, HA0, during maturation of the influenza virus. In part, because these molecules are tightly folded, the HA0 and the mature HA1 and HA2 differ slightly in their conformation and antigenic characteristics. Furthermore, the HA0 is more stable and resistant to denaturation and to proteolysis.

Isolation, propagation and purification of influenza viral strains in order to clone the desired HA genes may be performed by any method known in the art, including, for example, those disclosed in U.S. Pat. No. 5,762,939, incorporated by reference herein.

Recombinant HA antigens may be expressed in an appropriate host cell. For example, the recombinant HA can be expressed in microalgal cells, as disclosed in U.S. Patent Publication No. 2011/0189228, which is hereby incorporated by reference in its entirety. Alternatively, the recombinant HA can be expressed in insect cells. Other suitable host cells can be used to express recombinant HA, including, for example, mammalian cells, plant cells, or yeast cells.

In certain embodiments, the recombinant HA is expressed in insect cells infected with a viral-HA vectors, such as a baculovirus vector, as disclosed, for example, in U.S. Pat. No. 5,976,552, which is hereby incorporated by reference in its entirety. Baculovirus/insect cell cultures derived recombinant HA0 is known to confer protective immunity to influenza. Baculoviruses are DNA viruses in the family Baculoviridae. These viruses are known to have a narrow host-range that is limited primarily to the Lepidopteran species of insects (e.g., butterflies and moths). For example, the baculovirus Autographa californica Nuclear Polyhedrosis Virus (AcNPV) replicates efficiently in susceptible cultured insect cells. AcNPV has a double-stranded closed circular DNA genome of about 130,000 base pairs and is well-characterized with regard to host range, molecular biology, and genetics.

Many baculoviruses, including AcNPV, form large protein crystalline occlusions within the nucleus of infected cells. A single polypeptide, referred to as a polyhedrin, accounts for approximately 95% of the protein mass of these occlusion bodies. The gene for polyhedrin is present as a single copy in the AcNPV viral genome. Because the polyhedrin gene is not needed for virus replication in culture cells, it can be readily modified to express foreign genes. The foreign gene sequence may be inserted into the AcNPV gene just 3′ to the polyhedrin promotor sequence such that it is under the transcriptional control of the polyhedrin promoter. Recombinant baculoviruses, including recombinant baculoviruses encoding recombinant HA proteins, may then replicate in a variety of insect cell lines. Recombinant HA proteins may also be expressed in other expression vectors, including, for example, Entomopox viruses (the poxviruses of insects), cytoplasmic polyhedrosis viruses (CPV), and transformation of insect cells with the recombinant HA gene or genes.

The primary gene product is unprocessed, full-length HA (rHA0) and is not secreted but remains associated with peripheral membranes of infected cells. In insect cells, this rHA0 is glycosylated with N-linked, high-mannose type glycans, and there is evidence that rHA0 forms trimers post-translationally, which then accumulate in cytoplasmic cell membranes.

rHA0 can be selectively extracted from the peripheral membranes with a non-denaturing, non-ionic detergent or other methods known in the art for the purification of recombinant proteins from cells, e.g., insect cells, including, for example, affinity or gel chromatography, antigen binding, DEAE ion exchange, or lentil lectin affinity chromatography. The purified rHA0 may then be resuspended in an isotonic, buffered solution. In certain embodiments, the rHA0 is purified to at least about 80%, such as at least about 85%, at least about 90%, at least about 95%, at least about 96%, at least about 97%, at least about 98%, or at least about 99%.

The recombinant influenza virus HA disclosed herein can be formulated and packaged, alone or in combination with other recombinant influenza virus HA antigens and/or with recombinant influenza virus NA as discussed below. In certain embodiments, the recombinant influenza virus HA is formulated with one, two, three, four, five, six, or seven additional recombinant influenza virus HA antigens, and in certain embodiments, the recombinant influenza virus HA is formulated with one, two, three, four, five, six, or seven additional recombinant influenza virus NA antigens. In certain embodiments, the recombinant influenza virus HA is formulated with three additional recombinant influenza virus HA antigens to produce a quadrivalent vaccine or immunogenic composition. In certain embodiments, the recombinant influenza virus HA is formulated with three additional recombinant influenza virus HA antigens and four additional recombinant influenza virus NA antigens to produce an octavalent vaccine or immunogenic composition.

The recombinant influenza virus HAs present in the vaccine or immunogenic compositions disclosed herein may include any combination of recombinant influenza virus HA from standard of care influenza virus strains and/or machine learning influenza virus HA as disclosed herein. For example, in certain embodiments the recombinant influenza virus HA may be wild-type influenza HA, modified influenza HA, HA from seasonal or pandemic influenza virus strains, and/or influenza HA in any other form known in the art. In certain embodiments, disclosed herein is a recombinant influenza virus HA, wherein the HA is selected from an H1 HA from a standard of care influenza virus, an H3 HA from a standard of care influenza virus, an HA from a standard of care influenza virus strain from the B/Victoria lineage, or an HA from a standard of care influenza virus from the B/Yamagata lineage.

In certain embodiments disclosed herein, the recombinant influenza virus HA is from a pandemic strain or a strain with pandemic potential, including, for example, H1, H2, H3, H5, H7, H9, and/or H10.

In certain embodiments disclosed herein, the recombinant influenza virus HA is one or more machine learning recombinant influenza virus HA having a molecular sequence identified or designed from a machine learning model. In certain embodiments, the machine learning recombinant influenza virus HA may be selected from one or more of H1 HA, H3 HA, HA from a B/Victoria lineage, HA from a B/Yamagata lineage, or combinations thereof.

When selecting one or more machine learning influenza virus HAs, any machine learning algorithm may be used. For example, envisioned herein are any of the machine learning algorithms and methods disclosed in PCT Application Nos. WO 2021/080990 A1, entitled Systems and Methods for Designing Vaccines, and WO 2021/080999 A1, entitled Systems and Methods for Predicting Biological Responses, U.S. Provisional Application No. 63/319,692, entitled Machine-Learning Techniques in Protein Design for Vaccine Generation, and U.S. Provisional Application No. 63/319,700, entitled Machine-Learning Techniques in Protein Design for Vaccine Generation, all of which are incorporated by reference in their entireties herein.

In certain embodiments, a predictive machine learning model of influenza antigenicity may be constructed, allowing prediction of antibody titer in animal models and/or humans. In certain embodiments, a machine learning model may extract feature values from input data of a training set, the features being variables deemed potentially relevant to whether or not the input data items have the associated property or properties. An ordered list of the features for the input data may be referred to as the feature vector for the input data. In certain embodiments, the machine learning model applies dimensionality reduction (e.g., via linear discrimination analysis (LDA), principal component analysis (PCA), learned deep features from a neural network, or the like) to reduce the amount of data in the feature vectors for the input data to a smaller, more representative set of data. A set of influenza sequences to be protected against (e.g., target strains) may then be identified and a selection algorithm constructed.

Hemagglutinin activity may be measured using techniques known in the art, including, for example, hemagglutinin inhibition assay (HAI). An HAI applies the process of hemagglutination, in which sialic acid receptors on the surface of red blood cells (RBCs) bind to a hemagglutinin glycoprotein found on the surface of an influenza virus (and several other viruses) and create a network, or lattice structure, of interconnected RBCs and virus particles, referred to as hemagglutination, which occurs in a concentration dependent manner on the virus particles. This is a physical measurement taken as a proxy as to the facility of a virus to bind to similar sialic acid receptors on pathogen-targeted cells in the body. The introduction of anti-viral antibodies raised in a human or animal immune response to another virus (which may be genetically similar or different to the virus used to bind to the RBCs in the assay) interfere with the virus-RBC interaction and change the concentration of virus sufficient to alter the concentration at which hemagglutination is observed in the assay. One goal of an HAI can be to characterize the concentration of antibodies in the antiserum or other samples containing antibodies relative to their ability to inhibit hemagglutination in the assay. The highest dilution of antibody that prevents hemagglutination is called the HAI titer (i.e., the measured response).

Another approach to measuring a HA antibody response is to measure a potentially larger set of antibodies elicited by a human or animal immune response, which are not necessarily capable of affecting hemagglutination in the HAI assay. A common approach for this leverages enzyme-linked immunosorbent assay (ELISA) techniques, in which a viral antigen (e.g., hemagglutinin) is immobilized to a solid surface, and then antibodies from the antisera are allowed to bind to the antigen. The readout measures the catalysis of a substrate of an exogenous enzyme complexed to either the antibodies from the antisera, or to other antibodies which themselves bind to the antibodies of the antisera. Catalysis of the substrate gives rise to easily detectable products. There are many variations of this sort of in vitro assay. One such variation is called antibody forensics (AF), which is a multiplexed bead array technique that allowed a single sample of serum to be measured against many antigens simultaneously. These measurements characterize the concentration and total antibody recognition, as compared to HAI titers, which are taken to be more specifically related to interference with sialic acid binding by hemagglutinin molecules. Therefore, an antisera's antibodies may in some cases have proportionally higher or lower measurements than the corresponding HAI titer for one virus's hemagglutinin molecules relative to another virus's hemagglutinin molecules; in other words, these two measurements, AF and HAI, may not be linearly related.

Another method of measuring HA antibody response includes a viral neutralization assay (e.g., microneutralization assay), wherein an antibody titer is measured by a reduction in plaques, foci, and/or fluorescent signal, depending on the specific neutralization assay technique, in permissive cultured cells following incubation of virus with serial dilutions of an antibody/serum sample.

### Neuraminidase (NA)

Neuraminidase (NA), along with HA, is the second major influenza surface protein. NA removes sialic acid from cellular glycoproteins and glycolipids and from newly synthesized HA and NA on nascent virions. The removal of sialic acid by NA promotes the efficient release of viral particles from the surface of infected cells by preventing aggregation of viral particles. It also prevents virus from binding via HA to dying cells that have already been infected, promoting the further spread of the viral infection. When NA is present in immunogenic form either in a traditional vaccine or on the intact virion, it is a minority component and therefore subservient to continuing antigenic competition with the immunodominant HA. Due to competitive mechanisms, the immunogenic response to NA appears to be partially suppressed in favor of the more frequently occurring HA antigen (Johanssen et al., Immunologic response to influenza virus neuraminidase is influenced by prior experience with the associated viral hemagglutinin, J. IMMUNOL. 1987; 139(6):2010-2014; and Kilbourne, Comparative Efficacy of Neuraminidase-Specific and Conventional Influenza Virus Vaccines in Induction of Antibody to Neuraminidase in Humans, J. INFECT. Dis. 1976; 134(4):384-94). As a result, the effect of NA immunity is generally overshadowed by the neutralizing HA antibodies.

a. Wild-Type Influenza Virus Neuraminidase

The compositions and methods disclosed herein may, in certain embodiments, involve the use of tetrameric NA polypeptides that comprise four copies of a wild-type monomeric NA molecule. NA is a type II transmembrane glycoprotein that assembles on the virus surface as a tetramer of four identical monomers. The molecular mass of the wild-type monomer is typically about 55-72 kDa, depending on the influenza subtype; the molecular mass of the tetramer is typically about 240-260 kDa, depending on the influenza subtype. Each monomer consists of four distinct structural domains: the enzymatic head region, the stalk region, the transmembrane region, and the cytoplasmic tail. The largest domain is the head region, which is tethered to the viral membrane by a stalk region connected to the transmembrane region and finally the N-terminal cytoplasmic domain.

The stalk region among different influenza A virus subtypes, including N1 and N2, can vary significantly in size and amino acid structure (Blok et al., Variation in the membrane-insertion and ‘stalk’ sequences in eight subtypes of influenza type A virus neuraminidase, BIOCHEMISTRY 1982, 21(17):4001-4007). The differences in stalk length are thought to regulate the distance of the enzymatic head region and impact the ability of NA to access sialic acid on cell surface receptors, with shorter stalk regions correlating with reduced sialidase activity (Da Silva et al., Assembly of Subtype 1 Influenza Neuraminidase is Driven by Both the Transmembrane and Head Domains, J BIOL CHEM 2013, 288(1):644-53; and McAuley et al., Influenza Virus Neuraminidase Structure and Functions, FRONTIERS IN MICROBIOLOGY 2019, 10(39)). Notwithstanding the variability among stalk regions of different subtypes, NA stalk regions also share some structural features, including at least one cysteine residue and a potential glycosylation site. The cysteine residue(s) may be involved in the formation of disulfide bonds between NA monomers and assist in the formation of a stabilized NA tetramer, while the glycosylation site may contribute to tetramer stabilization (McAuley et al., 2019). For example, a conserved cysteine residue at amino acid position 78 of N2 NA is believed to play a role in the tetramer assembly mechanism (Shtyrya et al., Influenza virus neuraminidase: structure and function, ACTA NATURAE 2009; 1(2): 26-32).

The enzymatic head region is comprised of four monomers. Each monomer in the head forms a conserved six-bladed propeller structure. Each blade has four anti-parallel β-sheets that are stabilized by disulfide bonds and connected by loops of varying length (McAuley et al., 2019). Tetramerization of the monomers is important for the formation of the active site and synthesis of the enzymatically active NA (Dai et al., Identification of Residues That Affect Oligomerization and/or Enzymatic Activity of Influenza Virus H5N1 Neuraminidase Proteins, J. VIROLOGY 2016, 90(20):9457-70).

Although the amino acid sequence and length of NA can vary significantly between different influenza A virus NA subtypes, such as N1 and N2, and particularly the NA stalk regions of different influenza A virus NA subtypes, the amino acid sequence length of N2 from different influenza strains is typically about 469 amino acids, with a few strains having about one or two (or more) amino acid insertions or deletions, typically in the head region. When referring to specific amino acid residues in a wild type N2, the specific amino acid residue numbers are based on N2 numbering, as understood in the art. The N-terminal cytoplasmic tail typically corresponds to amino acid 1-6 of the wild type N2 sequence, while the transmembrane domain typically corresponds to amino acids 7-35 of the wild type N2 sequence. For example, in the wild type NA sequence of the strain A/PERTH/16/2009 (SEQ ID NO: 1), the cytoplasmic region corresponds to amino acids 1-6 of SEQ ID NO: 1, while the transmembrane region corresponds to amino acids 7-35 of SEQ ID NO: 1. The length of the N2 stalk region is typically about 46 amino acids in length, starting at about amino acid 36 and ending at about amino acid 82 of the wild type N2 sequence. For example, in the wild type NA sequence of the strain A/PERTH/16/2009 (SEQ ID NO: 1), the stalk region corresponds to amino acid 36 to about amino acid 82 of SEQ ID NO: 1. However, the precise boundary between the end of the N2 stalk region and the start of the N2 head region has not been resolved by x-ray crystallography.

b. Recombinant and/or Modified Influenza Virus Neuraminidase

The present methods and compositions of the disclosure involve the use of recombinant NA. In certain embodiments, the recombinant NA comprises four copies of a modified monomeric NA molecule that forms soluble, tetrameric NA when expressed in a host cell. In one aspect, the modified monomeric NA molecule includes a head region of an influenza virus NA and a heterologous oligomerization domain, but lacks at least a portion of one or more of a cytoplasmic tail, a transmembrane region, and a stalk region of the influenza virus NA.

For example, the modified monomeric NA may include a heterologous tetramerization domain that replaces one or more of a cytoplasmic tail, a transmembrane region, and a stalk region of the influenza virus NA or that replaces the cytoplasmic tail, the transmembrane region, and all or substantially all of the stalk region of the influenza virus NA. In certain embodiments, the heterologous tetramerization domain is a tetramerization domain, as disclosed, for example, in U.S. Patent Publication No. 2013/0034578, which is hereby incorporated by reference in its entirety. See also, Schmidt et al., PLos ONE, 2011, 6(2):e16284; Da Silva et al., J Biol Chem, 2013, 288(1):644-53; Dai et al., 2016, J. Virology, 90(20):9457-70; Bosch et al., 2010, J. Virology, 84(19):10366-74; Prevato et al., 2015, PLos ONE, 10(8): e0135474. In other embodiments, the heterologous tetramerization domain is a peptide found at the extreme C-terminus of lamprey VLR-B antibodies (i.e., the domain named “C-TERM” in FIG. 11C of PCT Publication No. WO 2008/016854, which is hereby incorporated by reference in its entirety) as described in PCT Publication No. WO 2016/097769, such as SEQ ID NO:1 or SEQ ID NO:2 of WO 2016/097769, which is hereby incorporated by reference in its entirety).

In certain embodiments, the modified monomeric influenza virus NA comprises a signal peptide, heterologous tetramerization domain, and a head region of an influenza virus NA, wherein expression of the modified monomeric influenza virus NA in a host cell results in the secretion of a tetrameric NA.

The wild type NA protein is a membrane bound protein that includes a transmembrane domain. To make a soluble NA protein, it is possible to delete the transmembrane domain and add a signal peptide. The signal peptide targets the recombinant NA protein to the secretory pathway so that the recombinant NA protein is secreted from the host cell in which the recombinant NA is expressed. When the modified monomeric NA nucleic acid is translated into a polypeptide inside the host cell, the polypeptide contains the signal peptide. However, during post-translational processing, the signal peptide is cleaved, such that the secreted polypeptide no longer contains the signal peptide. As such, although the modified monomeric NA may include a signal peptide following translation to target the modified monomeric NA to the secretory pathway, the signal peptide is removed through post-translational processing, such that soluble tetrameric NA obtained from host cells that express the modified monomeric NA are made up of four modified NA monomers that no longer contain the signal peptide.

In certain embodiments, for example, a tetrameric NA comprises four copies of a modified monomeric influenza virus NA, wherein the modified monomeric influenza virus NA comprises a head region of an influenza virus NA and a heterologous tetramerization domain.

In certain embodiments, the cytoplasmic tail, the transmembrane region and all or substantially all of the stalk region of the influenza virus NA may be replaced by the signal peptide and the heterologous tetramerization domain. The modified NA comprising a heterologous tetramerization domain can lack the entire NA stalk region, or it can lack substantially all of the NA stalk region, i.e., the modified NA construct can include a C-terminal portion of the NA stalk region. For example, the modified NA comprising a heterologous tetramerization domain can include about 1-13 of the most C-terminal amino acids of the NA stalk region. As understood in the art, the most C-terminal amino acids of the stalk region are those residues that are immediately adjacent to the NA head region. By way of further example, the modified NA comprising a heterologous tetramerization domain construct can include 1-10, 1-9, 1-8, 1-7, 1-6, 1-5, 1-4, 1-3, or 1-2 of the most C-terminal amino acids of the NA stalk region. By way of further example, the modified NA comprising a heterologous tetramerization domain construct can include about 8 of the most C-terminal amino acids of the NA stalk region.

In certain embodiments, the heterologous tetramerization domain is a Staphylothermus marinus tetrabrachion tetramerization domain, a GCN4 leucine zipper tetramerization domain, a tetramerization domain from a paramyxovirus phosphoprotein, or a human vasodilator stimulated phosphoprotein (VASP) tetramerization domain.

By way of further example, it has been discovered that modified monomeric influenza virus subtype 2 neuraminidase (N2) lacking all or substantially all of the stalk domain can form soluble tetrameric NA when expressed in cells, even without the addition of a heterologous tetramerization domain, as disclosed in International PCT Application No. PCT/US2022/039980, which is hereby incorporated by reference in its entirety. Although not all N2 strains lacking all or substantially all of the stalk domain produced soluble tetrameric NA in detectable amounts, the majority of N2 strains tested produced detectable amounts of soluble tetrameric NA, showing that a truncated stalk design strategy can be broadly applied to the NA protein from various N2 influenza strains. Depending on the N2 strain used, this modified monomeric NA design strategy may result in the production of predominately tetrameric NA or a mixture of monomeric NA and tetrameric when expressed in a host cell. Thus, certain N2 strains and certain stalk-deleted variants of specific N2 strains produce higher yields of soluble, tetrameric NA when expressed in cells. In either instance, it may be desirable to purify the tetrameric NA produced when such modified NA constructs are expressed in host cells.

As used herein “substantially all of a stalk region” of an influenza virus subtype 2 neuraminidase (N2) refers to amino acid 36 to at least amino acid 69 of the stalk region of an influenza virus N2. Thus, a modified N2 lacking the cytoplasmic tail, the transmembrane region, and substantially all of the stalk region may lack amino acids 1-70, 1-71, 1-72, 1-73, 1-74, 1-75, 1-76, 1-77, 1-78, 1-79, 1-80, or 1-81 of an influenza virus subtype 2 NA. Put another way, the modified N2 described herein can include up to 13 of the most C-terminal amino acids of the stalk region of the influenza virus subtype 2 NA, where the most C-terminal amino acids of the stalk region typically refer to amino acids 70-82 of the N2. In certain embodiments, the cytoplasmic tail, the transmembrane region, and the entire stalk region (e.g., amino acids 1-82) have been removed from the modified N2.

In some embodiments, for example, a tetrameric NA comprises four copies of a modified influenza virus subtype 2 neuraminidase in which the modified influenza virus neuraminidase comprises a head region of an influenza virus neuraminidase and lacks the cytoplasmic tail, the transmembrane region, and all or substantially all of the stalk region of the influenza virus neuraminidase, and wherein the tetrameric NA does not contain a heterologous tetramerization domain. In some of these embodiments, the cytoplasmic tail, transmembrane region and all or substantially all of the stalk region of the influenza virus neuraminidase have been replaced by the signal peptide. The signal peptide is normally cleaved during post-translational processing such that the secreted, NA polypeptide typically does not contain the signal peptide. In some of these embodiments, for example, amino acid 1 to at least amino acid 70-82 of a wild-type N2 influenza virus NA have been replaced by the signal peptide. These modified N2 constructs in which the cytoplasmic domain, the transmembrane domain and all or substantially all of the stalk region are replaced by a signal peptide and which form tetrameric NA when expressed in cells are also described in further detail in International PCT Application No. PCT/US2022/039980, which is hereby incorporated by reference in its entirety.

Tetrameric NA molecules formed by these modified monomeric NA are generally substantially soluble in fluidic samples and are also typically catalytically active (e.g., capable of enzymatically cleaving glycosidic linkages of neuraminic acids). However, tetrameric NA molecules may also be catalytically inactive, for example, due to a mutation.

Neuraminidase activity can be measured using techniques known in the art, including, for example, a MUNANA assay, ELLA assay, or an NA-Star® assay (ThermoFisher Scientific, Waltham, MA). In the MUNANA assay, 2′-(4-methylumbelliferyl)-alpha-D-N-acetylneuraminic acid (MUNANA) is used as a substrate. Any enzymatically active neuraminidase contained in the sample cleaves the MUNANA substrate, releasing 4-Methylumbelliferone (4-MU), a fluorescent compound. Thus, the amount of neuraminidase activity in a test sample correlates with the amount of 4-MU released, which can be measured using the fluorescence intensity (RFU, Relative Fluorescence Unit).

For purposes of determining the neuraminidase activity of a soluble tetrameric NA of the present disclosure, a MUNANA assay should be performed using the following conditions: mix soluble tetrameric NA with buffer [33.3 mM 2-(N-morpholino) ethanesulfonic acid (MES, pH 6.5), 4 mM CaCl₂, 50 mM BSA] and substrate (100 μM MUNANA) and incubate for 1 hour at 37° C. with shaking; stop the reaction by adding an alkaline pH solution (0.2M Na₂CO₃); measure fluorescence intensity, using excitation and emission wavelengths of 355 and 460 nm, respectively; and calculate enzymatic activity against a 4MU reference. If necessary, an equivalent assay can be used to measure neuraminidase enzymatic activity.

The recombinant influenza virus NAs present in the vaccine or immunogenic compositions disclosed herein may include any combination of recombinant influenza virus NA from standard of care influenza virus strains and/or machine learning influenza virus NA as disclosed herein. For example, in certain embodiments the recombinant influenza virus NA may be wild-type influenza NA, non-wild type influenza NA, NA from seasonal or pandemic influenza virus strains, and/or influenza NA in any other form known in the art. In certain embodiments, disclosed herein is a recombinant influenza virus NA, wherein the NA is selected from an N1 NA from a standard of care influenza virus, an N2 NA from a standard of care influenza virus, an NA from a standard of care influenza virus strain from the B/Victoria lineage, or an NA from a standard of care influenza virus from the B/Yamagata lineage.

In certain embodiments disclosed herein, the recombinant influenza virus NA is from a pandemic strain or a strain with pandemic potential, including, for example, N1, N2, N7, and/or N9.

In certain embodiments disclosed herein, the one or more recombinant influenza virus NA is identified or designed using a machine learning model (“recombinant machine learning influenza virus NA”). In certain embodiments, the machine learning recombinant influenza virus NA may be selected from one or more of N1 NA, N2 NA, NA from a B/Victoria lineage, NA from a B/Yamagata lineage, or combinations thereof.

As disclosed above for machine learning recombinant influenza virus HA, when selecting one or more machine learning recombinant influenza virus NAs, any machine learning algorithm may be used. For example, envisioned herein are any of the machine learning algorithms and methods disclosed in PCT Application Nos. WO 2021/080990 A1, entitled Systems and Methods for Designing Vaccines, and WO 2021/080999 A1, entitled Systems and Methods for Predicting Biological Responses, both of which are incorporated by reference in their entireties herein.

### Vaccine or Immunogenic Compositions

In certain aspects, disclosed herein is a vaccine or immunogenic composition comprising a plurality of recombinant influenza virus proteins comprising one or more (such as two, three, or four) recombinant influenza virus HA and one or more (such as two, three, or four) recombinant influenza virus NA. In certain embodiments, the one or more (such as two, three, or four) recombinant influenza virus HA are selected from an H1 HA, an H3 HA, an HA from the B/Victoria lineage, an HA from the B/Yamagata lineage, or a combination thereof. In certain embodiments, the one or more (such as two, three, or four) recombinant influenza virus NA comprising a heterologous tetramerization domain are selected from an N1 NA, an N2 NA, an NA from the B/Victoria lineage, an NA from the B/Yamagata lineage, or a combination thereof. In certain embodiments, the one or more recombinant influenza virus NA that lack the cytoplasmic tail, transmembrane region and all or substantially all of the stalk region of the influenza virus neuraminidase and that do not contain a heterologous tetramerization domain is an N2 NA.

In certain aspects, disclosed herein is a vaccine or immunogenic composition comprising a plurality of recombinant influenza virus proteins, wherein the plurality of recombinant influenza virus proteins comprises (1) a first recombinant influenza virus hemagglutinin (HA), wherein the first recombinant influenza virus HA is an H1 HA; (2) a second recombinant influenza virus HA, wherein the second recombinant influenza virus HA is an H3 HA; (3) a third recombinant influenza virus HA, wherein the third recombinant influenza virus HA is from a B/Victoria lineage; (4) a fourth recombinant influenza virus HA, wherein the fourth recombinant influenza virus HA is from a B/Yamagata lineage; (5) a first recombinant influenza virus neuraminidase (NA), wherein the first recombinant influenza virus NA is an N1 NA; (6) a second recombinant influenza virus NA, wherein the second recombinant influenza virus NA is an N2 NA; (7) a third recombinant influenza virus NA, wherein the third recombinant influenza virus NA is from a B/Victoria lineage; and (8) a fourth recombinant influenza virus NA, wherein the fourth recombinant influenza virus NA is from a B/Yamagata lineage. In various embodiments, one of more of the recombinant NA is modified as described herein.

Further disclosed herein are vaccine or immunogenic compositions comprising a plurality of recombinant influenza virus proteins, wherein the plurality of recombinant influenza virus proteins consists of (1) a first recombinant influenza virus hemagglutinin (HA), wherein the first recombinant influenza virus HA is an H1 HA; (2) a second recombinant influenza virus HA, wherein the second recombinant influenza virus HA is an H3 HA; (3) a third recombinant influenza virus HA, wherein the third recombinant influenza virus HA is from a B/Victoria lineage; (4) a fourth recombinant influenza virus HA, wherein the fourth recombinant influenza virus HA is from a B/Yamagata lineage; (5) a first recombinant influenza virus neuraminidase (NA), wherein the first recombinant influenza virus NA is an N1 NA; (6) a second recombinant influenza virus NA, wherein the second recombinant influenza virus NA is an N2 NA; (7) a third recombinant influenza virus NA, wherein the third recombinant influenza virus NA is from a B/Victoria lineage; and (8) a fourth recombinant influenza virus NA, wherein the fourth recombinant influenza virus NA is from a B/Yamagata lineage. In various embodiments, one of more of the recombinant NA is modified as described herein.

In certain embodiments, one or more (such as one, two, three, or four) of the recombinant influenza virus HA in the vaccine or immunogenic composition are from standard of care influenza strains, and in certain embodiments, each of the recombinant influenza virus HA in the vaccine or immunogenic composition is from a standard of care influenza strain. In certain embodiments, one or more (such as one, two, three, or four) of the recombinant influenza virus NA in the vaccine or immunogenic composition are from standard of care influenza strains, and in certain embodiments, each of the recombinant influenza virus NA in the vaccine or immunogenic composition is from a standard of care influenza strain.

In certain embodiments, the vaccine or immunogenic composition comprises an H1 HA from an H1N1 influenza virus strain. In certain embodiments, the vaccine or immunogenic composition comprises an H3 HA from an H3N2 influenza virus strain. In certain embodiments, the vaccine or immunogenic composition comprises an N1 NA from an H1N1 influenza virus strain. In certain embodiments, the vaccine or immunogenic composition comprises an N2 NA from an N3N2 influenza virus strain. In certain embodiments, the vaccine or immunogenic composition comprises an H1 HA and a N1 NA from the same H1N1 influenza virus strain, and in certain embodiments, the vaccine or immunogenic composition comprises an H3 HA and an N2 NA from the same 1-13N2 influenza virus strain. In certain embodiments, the vaccine or immunogenic composition comprises an H1 HA and a N1 NA from different H1N1 influenza virus strains, and in certain embodiments, the vaccine or immunogenic composition comprises an H3 HA and an N2 NA from different H3N2 influenza virus strains.

In certain embodiments, the vaccine or immunogenic composition comprises an H1 HA from an H1N1 influenza virus strain, an H3 HA from an H3N2 influenza virus strain, an N1 NA from an H1N1 influenza virus strain, and a N2 NA from an H3N2 influenza virus strain. In certain embodiments, the vaccine or immunogenic composition comprises an H1 HA and a N1 NA from the same H1N1 influenza virus strain, and in certain embodiments, the vaccine or immunogenic composition comprises an H3 HA and an N2 NA from the same H3N2 influenza virus strain. In certain embodiments, the vaccine or immunogenic composition comprises an H1 HA and a N1 NA from different H1N1 influenza virus strains, and in certain embodiments, the vaccine or immunogenic composition comprises an H3 HA and an N2 NA from different 1-3N2 influenza virus strains.

One or more of the recombinant influenza virus HA and one or more of the recombinant influenza virus NA in the multivalent vaccine or immunogenic composition may be formulated and packaged alone or in combination with other recombinant HA and/or NA antigens. In certain embodiments, the recombinant influenza virus HA is formulated with one, two, or three additional recombinant influenza virus HA antigens, such as one, two, or three additional recombinant antigens from standard of care influenza virus strains. In certain embodiments, the recombinant influenza virus HA is formulated with three additional recombinant influenza virus HA antigens to produce a quadrivalent vaccine or immunogenic composition.

In certain embodiments, the recombinant influenza virus NA is formulated with one, two, or three additional recombinant influenza virus NA antigens, such as one, two, or three additional recombinant antigens from standard of care influenza virus strains. In certain embodiments, the recombinant influenza virus NA is formulated with three additional recombinant influenza virus NA antigens to produce a quadrivalent vaccine or immunogenic composition.

In certain embodiments, the one or more, such as one, two, or three recombinant influenza virus NA is formulated with one or more, such as one, two, three, or four of the recombinant influenza virus HA. In certain embodiments, the vaccine or immunogenic composition may contain four recombinant influenza virus HA antigens and four recombinant influenza virus NA antigens to produce an octavalent vaccine or immunogenic composition. In certain embodiments, the four recombinant influenza virus HA antigens and the four recombinant influenza virus NA antigens may each be from a standard of care influenza virus strain. In certain embodiments, the octavalent vaccine or immunogenic composition comprising four recombinant influenza virus HA antigens and four recombinant influenza virus NA antigens further comprises one or more machine learning influenza virus HA and/or one or more machine learning influenza virus NA.

In certain embodiments, the recombinant influenza virus H1 HA, the recombinant influenza virus H3 HA, the recombinant influenza virus HA from the B/Victoria lineage, the recombinant influenza virus HA from the B/Yamagata lineage, the recombinant influenza virus N1 NA, the recombinant influenza virus N2 NA, the recombinant influenza virus NA from the B/Victoria lineage, and/or the recombinant influenza virus NA from the B/Yamagata lineage has a molecular sequence identified or designed from a machine learning model.

In certain embodiments, the vaccine or immunogenic composition is a pentavalent vaccine or immunogenic composition comprising one or more recombinant HA and one or more recombinant NA. In certain embodiments, the vaccine or immunogenic composition is a hexavalent vaccine or immunogenic composition comprising one or more recombinant HA and one or more recombinant NA. In certain embodiments, the vaccine or immunogenic composition is a heptavalent vaccine or immunogenic composition comprising one or more recombinant HA and one or more recombinant NA. In certain embodiments, the vaccine or immunogenic composition is an octavalent vaccine or immunogenic composition comprising one or more recombinant HA and one or more recombinant NA, such as four recombinant HA and four recombinant NA. In certain embodiments, the vaccine or immunogenic composition is a multivalent vaccine or immunogenic composition comprising more than 8 different HA and NA molecules.

Each recombinant HA may be present in the compositions disclosed herein in an amount effective to induce an immune response in a subject to which the composition is administered. In certain embodiments, each recombinant HA may be present in the vaccine or immunogenic compositions disclosed herein in an amount ranging, for example, from about 0.1 μg to about 500 μg, such as from about 5 μg to about 120 μg, from about 1 μg to about 60 μg, from about 10 μg to about 60 μg, from about 15 μg to about 60 μg, from about 40 μg to about 50 μg, from about 42 μg to about 47 μg, from about 5 μg to about 45 μg, from about 15 μg to about 45 μg, from about 0.1 μg to about 90 μg, from about 5 μg to about 90 μg, from about 10 μg to about 90 μg, or from about 15 μg to about 90 μg. In certain embodiments, each recombinant HA may be present in the vaccine or immunogenic compositions disclosed herein in an amount of about 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, or about 90 μg.

Each recombinant NA may be present in the compositions disclosed herein in an amount effective to induce an immune response in a subject to which the composition is administered. In certain embodiments, each recombinant NA may be present in the vaccine or immunogenic compositions disclosed herein in an amount ranging, for example, from about 1 μg to about 500 μg, such as from about 5 μg to about 120 μg, from about 1 μg to about 60 μg, from about 10 μg to about 60 μg, from about 15 μg to about 60 μg, from about 5 μg to about 45 μg, from about 15 μg to about 45 μg, from about 0.1 μg to about 90 μg, from about 5 μg to about 90 μg, from about 10 μg to about 90 μg, from about 15 μg to about 90 μg, from about 5 μg to about 25 μg, or from about 10 μg to about 20 μg, or from about 12 μg to 18 μg. In certain embodiments, each recombinant NA may be present in the vaccine or immunogenic compositions disclosed herein in an amount of about 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, or about 90 μg.

In certain embodiments, the total amount of recombinant influenza HA and NA present in the vaccine or immunogenic compositions disclosed herein may range from about 150 μg to about 400 μg, from about 150 μg to about 300 μg, from about 200 μg to about 300 μg, from about 200 μg to about 250 μg, or from about 225 μg to about 245 μg. In certain embodiments, the total amount of recombinant influenza HA and NA present in the vaccine or immunogenic compositions disclosed herein is no more than about 500 μg, 400 μg, 350 μg, 300 μg, 250 μg, 200 μg, or 150 μg. In certain embodiments, the total amount of recombinant influenza HA and NA present in the vaccine or immunogenic compositions disclosed herein is about 500 μg, about 400 μg, about 350 μg, about 300 μg, about 290 μg, about 280 μg, about 270 μg, about 260 μg, about 250 μg, about 240 μg, about 230 μg, about 220 μg, about 210 μg, about 200 μg, about 190 μg, about 180 μg, about 170 μg, about 160 μg, or about 150 μg.

The vaccine or immunogenic composition can also further comprise an adjuvant. As used herein, the term “adjuvant” refers to a substance or vehicle that non-specifically enhances the immune response to an antigen. Adjuvants can include a suspension of minerals (alum, aluminum salts, including, for example, aluminum hydroxide/oxyhydroxide (AlOOH), aluminum phosphate (AlPO₄), aluminum hydroxyphosphate sulfate (AAHS) and/or potassium aluminum sulfate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as lipids and costimulatory molecules. Exemplary biological adjuvants include AS04 (Didierlaurent, A. M. et al, AS04, an Aluminum Salt-and TLR4 Agonist-Based Adjuvant System, Induces a Transient Localized Innate Immune Response Leading to Enhanced Adaptive Immunity, J. IMMUNOL. 2009, 183: 6186-6197), IL-2, RANTES, GM-CSF, TNF-α, IFN-γ, G-CSF, LFA-3, CD72, B7-1, B7-2, OX-40L and 41 BBL.

In certain embodiments, the adjuvant is a squalene-based adjuvant comprising an oil-in-water adjuvant emulsion comprising at least: squalene, an aqueous solvent, a polyoxyethylene alkyl ether hydrophilic nonionic surfactant, and a hydrophobic nonionic surfactant. In certain embodiments, the emulsion is thermoreversible, optionally wherein 90% of the population by volume of the oil drops has a size less than 200 nm.

In certain embodiments, the polyoxyethylene alkyl ether is of formula CH₃—(CH₂)ₓ—(O—CH₂—CH₂)ₙ—OH, in which n is an integer from 10 to 60, and x is an integer from 11 to 17. In certain embodiments, the polyoxyethylene alkyl ether surfactant is polyoxyethylene(12) cetostearyl ether.

In certain embodiments, 90% of the population by volume of the oil drops has a size less than 160 nm. In certain embodiments, 90% of the population by volume of the oil drops has a size less than 150 nm. In certain embodiments, 50% of the population by volume of the oil drops has a size less than 100 nm. In certain embodiments, 50% of the population by volume of the oil drops has a size less than 90 nm.

In certain embodiments, the adjuvant further comprises at least one alditol, including, but not limited to, glycerol, erythritol, xylitol, sorbitol and mannitol.

In certain embodiments the hydrophilic/lipophilic balance (HLB) of the hydrophilic nonionic surfactant is greater than or equal to 10. In certain embodiments, the HLB of the hydrophobic nonionic surfactant is less than 9. In certain embodiments, the HLB of the hydrophilic nonionic surfactant is greater than or equal to 10 and the HLB of the hydrophobic nonionic surfactant is less than 9.

In certain embodiments, the hydrophobic nonionic surfactant is a sorbitan ester, such as sorbitan monooleate, or a mannide ester surfactant. In certain embodiments, the amount of squalene is between 5 and 45%. In certain embodiments, the amount of polyoxyethylene alkyl ether surfactant is between 0.9 and 9%. In certain embodiments, the amount of hydrophobic nonionic surfactant is between 0.7 and 7%. In certain embodiments, the adjuvant comprises: i) 32.5% of squalene, ii) 6.18% of polyoxyethylene(12) cetostearyl ether, iii) 4.82% of sorbitan monooleate, and iv) 6% of mannitol.

In certain embodiments, the adjuvant further comprises an alkylpolyglycoside and/or a cryoprotective agent, such as a sugar, in particular dodecylmaltoside and/or sucrose.

In certain embodiments, the adjuvant comprises AF03, as described in Klucker et al., AF03, an alternative squalene emulsion-based vaccine adjuvant prepared by a phase inversion temperature method, J. PHARM. Sc1. 2012, 101(12):4490-4500, which is hereby incorporated by reference in its entirety. In certain embodiments, the adjuvant comprises a liposome-based adjuvant, such as SPA14, as described for example in WO 2022/090359, which is hereby incorporated by reference in its entirety. SPA14 is a liposome-based adjuvant containing a toll-like receptor 4 (TLR4) agonist (E6020) and saponin (QS21).

In addition to the recombinant HAs, recombinant NAs, and optional adjuvant, the vaccine or immunogenic composition may also further comprise one or more pharmaceutically acceptable excipients. In general, the nature of the excipient will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, vaccine or immunogenic compositions to be administered can contain minor amounts of non-toxic auxiliary substances, such as wetting or emulsifying agents, pharmaceutically acceptable salts to adjust the osmotic pressure, preservatives, stabilizers, buffers, sugars, amino acids, and pH buffering agents and the like, for example sodium acetate or sorbitan monolaurate.

Typically, the vaccine or immunogenic composition is a sterile, liquid solution formulated for parenteral administration, such as intravenous, subcutaneous, intraperitoneal, intradermal, or intramuscular. The vaccine or immunogenic composition may also be formulated for intranasal or inhalation administration. The vaccine or immunogenic composition can also be formulated for any other intended route of administration.

In some embodiments, a vaccine or immunogenic composition is formulated for intradermal injection, intranasal administration or intramuscular injection. In some embodiments, injectables are prepared in conventional forms, either as liquid solutions or suspensions, solid forms suitable for solution or suspension in liquid prior to injection, or as emulsions. In some embodiments, injection solutions and suspensions are prepared from sterile powders or granules. General considerations in the formulation and manufacture of pharmaceutical agents for administration by these routes may be found, for example, in Remington's Pharmaceutical Sciences, 19^{th} ed., Mack Publishing Co., Easton, P A, 1995; incorporated herein by reference. At present the oral or nasal spray or aerosol route (e.g., by inhalation) are most commonly used to deliver therapeutic agents directly to the lungs and respiratory system. In some embodiments, the vaccine or immunogenic composition is administered using a device that delivers a metered dosage of the vaccine or immunogenic composition. Suitable devices for use in delivering intradermal pharmaceutical compositions described herein include short needle devices such as those described in U.S. Pat. Nos. 4,886,499, 5,190,521, 5,328,483, 5,527,288, 4,270,537, 5,015,235, 5,141,496, 5,417,662 (all of which are incorporated herein by reference). Intradermal compositions may also be administered by devices which limit the effective penetration length of a needle into the skin, such as those described in WO 1999/34850, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Jet injection devices are described for example in U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, WO 1997/37705, and WO 1997/13537 (all of which are incorporated herein by reference). Also suitable are ballistic powder/particle delivery devices which use compressed gas to accelerate vaccine in powder form through the outer layers of the skin to the dermis. Additionally, conventional syringes may be used in the classical Mantoux method of intradermal administration.

Preparations for parenteral administration typically include sterile aqueous or nonaqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

### Kits

Further disclosed herein are kits for the vaccine or immunogenic compositions as disclosed herein. Kits may include a suitable container comprising the vaccine or immunogenic composition or a plurality of containers comprising different components of the vaccine or immunogenic composition, optionally with instructions for use.

In certain embodiments, the kit may comprise a plurality of containers, including, for example, a first container comprising one or more recombinant influenza virus HA as disclosed herein and a second container comprising one or more recombinant influenza virus NA as disclosed herein. For example, in certain embodiments, disclosed herein is a kit comprising (1) a first container comprising a first recombinant influenza virus HA, wherein the first recombinant influenza virus HA is an H1 HA; a second recombinant influenza virus HA, wherein the second recombinant influenza virus HA is an H3 HA; a third recombinant influenza virus HA, wherein the third recombinant influenza virus HA is from a B/Victoria lineage; a fourth recombinant influenza virus HA, wherein the fourth recombinant influenza virus HA is from a B/Yamagata lineage; and (2) a second container comprising a first recombinant influenza virus NA, wherein the first recombinant influenza virus NA is an N1 NA; a second recombinant influenza virus NA, wherein the second recombinant influenza virus NA is an N2 NA; a third recombinant influenza virus NA, wherein the third recombinant influenza virus NA is from a B/Victoria lineage; and a fourth recombinant influenza virus NA, wherein the fourth recombinant influenza virus NA is from a B/Yamagata lineage. In certain embodiments, the kit may further comprise a third container comprising an optional adjuvant, and in certain embodiments, the first and/or second container may comprise an optional adjuvant in addition to the recombinant influenza virus antigens.

In certain embodiments, the kit may comprise a single container comprising each of the one or more recombinant influenza virus HA as disclosed herein and each of the one or more recombinant influenza virus NA as disclosed herein, as well as an optional adjuvant. In certain embodiments, the optional adjuvant may be in a separate container.

The instructions for use may indicate that the contents of the first and second container can be combined prior to administration or that the contents of the first and second container are not combined and are administered separately.

### Nucleic Acids, Cloning, and Expression Systems

The present disclosure further provides artificial nucleic acid molecules encoding the disclosed recombinant HAs and NAs. The nucleic acids may comprise DNA or RNA and may be wholly or partially synthetic or recombinant. Reference to a nucleotide sequence as set out herein encompasses a DNA molecule with the specified sequence and encompasses an RNA molecule with the specified sequence in which U is substituted for T, or a derivative thereof, such as pseudouridine, unless context requires otherwise. Other nucleotide derivatives or modified nucleotides can be incorporated into the artificial nucleic acid molecules encoding the disclosed HAs and NAs.

The present disclosure also provides constructs in the form of a vector (e.g., plasmids, phagemids, cosmids, transcription or expression cassettes, artificial chromosomes, etc.) comprising an artificial nucleic acid molecule encoding a HA or NA as disclosed herein. The disclosure further provides a host cell which comprises one or more constructs as above.

Also provided are methods of making the recombinant HA or recombinant NA polypeptides using recombinant techniques known in the art and as discussed above. The production and expression of recombinant proteins is well known in the art and can be carried out using conventional procedures, such as those disclosed in Sambrook et al., Molecular Cloning: A Laboratory Manual (4th Ed. 2012), Cold Spring Harbor Press. For example, expression of the HA or NA polypeptide may be achieved by culturing under appropriate conditions host cells containing the artificial nucleic acid molecule encoding the HA or NA as disclosed herein. For example, expression of the recombinant HA or NA polypeptide may be achieved by culturing under appropriate conditions host cells containing the nucleic acid molecule encoding the HA or NA as disclosed herein. Following production by expression, the HA or NA may be isolated and/or purified using any suitable technique, then used as appropriate.

Systems for cloning and expression of a polypeptide in a variety of different host cells are well known in the art. Any protein expression system (e.g., stable or transient) compatible with the constructs disclosed in this application may be used to produce the HAs or NAs described herein.

Suitable vectors can be chosen or constructed, so that they contain appropriate regulatory sequences, including promoter sequences, terminator sequences, polyadenylation sequences, enhancer sequences, marker genes and other sequences as appropriate.

For expressing recombinant HA and/or recombinant NA as disclosed herein, nucleic acids encoding HA or nucleic acids encoding NA can be introduced into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. These techniques are well known in the art. (See, e.g., “Current Protocols in Molecular Biology,” Ausubel et al. eds., John Wiley & Sons, 2010). DNA introduction may be followed by a selection method (e.g., antibiotic resistance) to select cells that contain the vector.

The host cell may be a plant cell, a yeast cell, or an animal cell. Animal cells encompass invertebrate (e.g., insect cells), non-mammalian vertebrate (e.g., avian, reptile and amphibian) and mammalian cells. In one embodiment, the host cell is a mammalian cell. Examples of mammalian cells include, but are not limited to COS-7 cells, HEK293 cells; baby hamster kidney (BHK) cells; Chinese hamster ovary (CHO) cells; mouse sertoli cells; African green monkey kidney cells (VERO); human cervical carcinoma cells (e.g., HeLa); canine kidney cells (e.g., MDCK), and the like. In one embodiment, the host cells are CHO cells. In one embodiment, the host cells are insect cells.

### Machine Learning

When selecting one or more machine learning influenza virus HAs and/or NAs, any machine learning algorithm may be used. For example, envisioned herein are any of the machine learning algorithms and methods disclosed in PCT Application Nos. WO 2021/080990 A1, entitled Systems and Methods for Designing Vaccines, and WO 2021/080999 A1, entitled Systems and Methods for Predicting Biological Responses, U.S. Provisional Application No. 63/319,692, entitled Machine-Learning Techniques in Protein Design for Vaccine Generation, and U.S. Provisional Application No. 63/319,700, entitled Machine-Learning Techniques in Protein Design for Vaccine Generation, all of which are incorporated by reference in their entireties herein.

In certain embodiments, a predictive machine learning model of influenza antigenicity may be constructed, allowing prediction of antibody titer in animal models and/or humans. In certain embodiments, a machine learning model may extract feature values from input data of a training set, the features being variables deemed potentially relevant to whether or not the input data items have the associated property or properties. An ordered list of the features for the input data may be referred to as the feature vector for the input data. In certain embodiments, the machine learning model applies dimensionality reduction (e.g., via linear discrimination analysis (LDA), principal component analysis (PCA), learned deep features from a neural network, or the like) to reduce the amount of data in the feature vectors for the input data to a smaller, more representative set of data. A set of influenza sequences to be protected against (e.g., target strains) may then be identified and a selection algorithm constructed.

In certain embodiments, a system for designing vaccines is provided. The system includes one or more processors. The system includes computer storage storing executable computer instructions in which, when executed by one or more processors, cause the one or more processors to perform one or more operations. The one or more operations include applying, to a first temporal sequence data set, a plurality of driver models configured to generate output data representing one or more molecular sequences, the first temporal sequence data set indicating one or more molecular sequences and, for each of the one or more molecular sequences, one or more times of circulation for pathogenic strains including that molecular sequence as a natural antigen. The one or more operations include for each of the plurality of driver models, training the driver model by: i) receiving, from the driver model, output data representing one or more predicted molecular sequences based on the received first temporal sequence data set; ii) applying, to the output data representing the predicted one or more molecular sequences, a translational model configured to predict a biological response to molecular sequences for a plurality of translational axes to generate first translational response data representing one or more first translational responses corresponding to a particular translational axis of the plurality of translational axes based on the one or more predicted molecular sequences of the output data; iii) adjusting one or more parameters of the driver model based on the first translational response data; and iv) repeating steps i-iii for a number of iterations to generate trained translational response data representing one or more trained translational responses corresponding to the particular translational axis. The one or more operations include selecting, based on the one or more trained translational responses, a set of trained driver models of the plurality of driver models. The one or more operations include for each trained driver model of the set of trained driver models: applying, to a second temporal sequence data set, the trained driver model to generate trained output data representing one or more predicted molecular sequences for a particular season; applying, to the final output data, the translational model to generate second translational response data representing, for each translational axis of the plurality of translational axes, one or more second translational responses; and selecting, based on the second translational response data, a subset of trained driver models of the set of trained driver models.

At least one of the plurality of driver models can include a recurrent neural network. At least one of the plurality of driver models includes a long short-term memory recurrent neural network.

The output data representing one or more predicted molecular sequences based on the received first temporal sequence data set can include output data representing an antigen for each of a plurality of pathogenic seasons. The output data representing an antigen for each of a plurality of pathogenic seasons can include an antigen determined by predicting molecular sequences that will generate a maximized aggregate biological response across all pathogenic strains in circulation for a particular season. The output data representing an antigen for each of a plurality of pathogenic seasons can include an antigen determined by predicting molecular sequences that will generate a response that will effectively immunize against a maximized number of viruses in circulation for a particular season.

The plurality of translational axes can include at least one of a: ferret antibody forensics (AF) axis, ferret hemagglutination inhibition assay (HAI) axis, mouse AF axis, mouse HAI axis, human Replica AF axis, human AF axis, or human HAI axis. The number of iterations can be based on a predetermined number of iterations. The number of iterations can be based on a predetermined error value. The one or more first translational responses can include at least one of: a predicted ferret HAI titer, a predicted ferret AF titer, a predicted mouse AF titer, a predicted mouse HAI titer, a predicted human replica AF titer, a predicted human AF titer, or a predicted human HAI titer.

Selecting the set of trained driver models of the plurality of driver models can include assigning each driver model of the plurality of driver models to a class of driver models, wherein each class is associated with the particular translational axis of the plurality of translational axes used to train that driver model. Selecting the set of trained driver models of the plurality of driver models can include comparing, for each driver model of the plurality of driver models, the one or more trained translational responses of that driver model with the one or more trained translational responses of at least one other driver model assigned to the same class as that driver model.

The operations can further include for each trained driver model of the subset of trained driver models: validating that trained driver model by comparing the second translational response data corresponding to that trained driver model with observed experimental response data; and generating, in response to validating that trained driver model, a vaccine that includes the one or more molecular sequences represented by the trained output data corresponding to that trained driver model.

In an aspect, a system is provided. The system includes a computer-readable memory comprising computer-executable instructions. The system includes at least one processor configured to execute executable logic including at least one machine learning model trained to predict one or more molecular sequences, in which when the at least one processor is executing the computer-executable instructions, the at least one processor is configured to carry out one or more operations. The one or more operations include receiving temporal sequence data indicating one or more molecular sequences and, for each of the one or more molecular sequences, one or more times of circulation for pathogenic strains including that molecular sequence as a natural antigen. The one or more operations include processing the temporal sequence data through one or more data structures storing one or more portions of executable logic included in the machine learning model to predict one or more molecular sequences based on the temporal sequence data.

Predicting one or more molecular sequences based on the temporal sequence data can include predicting one or more immunological properties the predicted one or more molecular sequences will confer for use at a future time. Predicting the one or more molecular sequences based on the temporal sequence data can include predicting one or more molecular sequences that will generate a maximized aggregate biological response across all pathogenic strains of the temporal sequence data. Predicting the one or more molecular sequences based on the temporal sequence data can include predicting one or more molecular sequences that will generate a biological response that will effectively cover a maximized number of pathogenic strains of the temporal sequence data. The predicted one or more molecular sequences can be used to design a vaccine for pathogenic strains circulating during a time subsequent to the one or more times of circulation of the temporal sequence data.

The machine learning model can include a recurrent neural network.

In certain embodiments, a data processing system for predicting biological responses is provided. The system includes a computer-readable memory comprising computer-executable instructions. The system includes at least one processor configured to execute executable logic including at least one machine learning model trained to predict biological responses, wherein when the at least one processor is executing the computer-executable instructions, the at least one processor carries out one or more operations. The one or more operations include receiving first sequence data of a first molecular sequence. The one or more operations include receiving second sequence data of a second molecular sequence. The one or more operations include predicting a biological response for the second molecular sequence based at least partly on the received first and second sequence data.

The one or more operations can include receiving non-human biological response data corresponding with the first molecular sequence and the second molecular sequence. The one or more operations can include predicting the biological response is further based at least partly on the non-human biological response data. The one or more operations can include encoding the first sequence data and the second sequence data as amino acid mismatches.

The first molecular sequence can include a candidate antigen. The second molecular sequence can include a known viral strain.

Predicting the biological response can include predicting a human biological response. Predicting the biological response can include predicting at least one human biological response and at least one non-human biological response. The biological response can include an antibody titer. The machine learning model can include a deep neural network.

Machine learning techniques can be used to train a machine learning model to predict biological responses, such that incidences of false positives and false negatives are reduced. At least some of the systems and methods described can be used to, when compared with conventional techniques, efficiently process inherently sparse data, for example, by reducing the dirnensionality of the data. At least some of the described systems and methods can leverage non-linear relationships in received data to increase prediction accuracy relative to traditional techniques. At least some of the described systems and methods described can be used to simultaneously predict human biological responses and non-human biological responses. At least some of the described systems and methods can be used to predict experimentally unobserved outcomes.

In certain embodiments, a system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method for manufacturing a vaccine by using a continuous-data algorithm. The method includes receiving a discrete-data object that may include a plurality of first discrete values, the discrete-data object may include one or more amino acid sequences. The method also includes converting the discrete-data object into a continuous-data object that may include a plurality of first continuous values. The method also includes applying, to the continuous-data object, a continuous-data algorithm to generate a continuous-result object that may include a plurality of second continuous values. The method also includes converting the continuous-result object into a discrete-result object that may include a plurality of second discrete values. The method also includes manufacturing a vaccine that may include at least one of i) a protein defined by the discrete-result object, ii) a nucleic acid capable of producing the protein defined by the discrete-result object, and a iii) delivery vehicle capable of producing the protein defined by the discrete-result object. Other embodiments of this aspect include corresponding computer systems, apparatus, and computer programs recorded on one or more computer storage devices, each configured to perform the actions of the methods.

Implementations may include one or more of the following features. The method where the one or more amino acid sequences may include: a first amino acid sequence and a second amino acid sequence, each of the first and the second amino acid sequences including respective single letters or respective letter strings. Converting the discrete-data object into the continuous-data object may include: generating, for each first discrete value, a weight-vector of weight values, each weight value representing a likelihood that the first discrete value represents a particular amino acid; generating, for each weight value of each weight-vector, a property-vector of property values, each property value representing a physiochemical property of a particular amino acid; and combining the weight-vector and the property-vector to create the first continuous values of the continuous-data object. Each weight-vector has twenty weight values, each weight value corresponding to one of twenty possible amino acids. Converting the continuous-result object into the discrete-result object may include determining, for each second continuous value, a respective single amino acid, where the determined single amino acids form the plurality of second discrete values. The method further may include: generating a plurality of candidate discrete-result objects; and excluding, from the plurality of candidate discrete-result objects, at least one discrete-result object that specifies an amino acid failing a manufacturability test. Applying the continuous-data algorithm to generate the continuous-result object may include applying a gradient descent with a loss function that determines a loss-value based on a plurality of loss criteria, the loss function may include: a first loss criteria based on an immunological response given two amino acid sequences; a second loss criteria that modifies the loss-value for sub-sequences not found in a dataset of wildtype sequences or sub-sequences not predicted to fold correctly; and a third loss criteria that, for each weight-vector, modifies the loss-value based on the greatest value in the second continuous values. Implementations of the described techniques may include hardware, a method or process, or computer software on a computer-accessible medium.

One general aspect includes a system for generating amino acid sequences, which system may include computer memory. The system may also include one or more processors. The system may also include computer-memory storing instructions that, when executed by the processors, cause the processors to perform operations that may include: receiving a discrete-data object comprising a plurality of first discrete values, the discrete-data object comprising one or more amino acid sequences; converting the discrete-data object into a continuous-data object comprising a plurality of first continuous values; applying, to the continuous-data object, a continuous-data algorithm to generate a continuous-result object comprising a plurality of second continuous values; converting the continuous-result object into a discrete-result object comprising a plurality of second discrete values; and manufacturing a vaccine comprising at least one of i) a protein defined by the discrete-result object, ii) a nucleic acid capable of producing the protein defined by the discrete-result object, and iii) a delivery vehicle capable of producing the protein defined by the discrete-result object. Other embodiments of this aspect include corresponding computer systems, apparatus, and computer programs recorded on one or more computer storage devices, each configured to perform the actions of the methods.

Implementations may include one or more of the following features. In one embodiment, there is a system where the one or more amino acid sequences may include: a first amino acid sequence and a second amino acid sequence, each of the first and the second amino acid sequences including respective single letters or respective letter strings. Converting the discrete-data object into the continuous-data object may include: generating, for each first discrete value, a weight-vector of weight values, each weight value representing a likelihood that the first discrete value represents a particular amino acid; generating, for each weight value of each weight-vector, a property-vector of property values, each property value representing a physiochemical property of a particular amino acid; and combining the weight-vector and the property-vector to create the first continuous values of the continuous-data object. Each weight-vector has twenty weight values, each weight value corresponding to one of twenty possible amino acids. Converting the continuous-result object into the discrete-result object may include determining, for each second continuous value, a respective single amino acid, where the determined single amino acids form the plurality of second discrete values. The operations further may include: generating a plurality of candidate discrete-result objects; and excluding, from the plurality of candidate discrete-result objects, at least one discrete-result object that specifies an amino acid failing a manufacturability test. Applying the continuous-data algorithm to generate the continuous-result object may include applying a gradient descent with a loss function that determines a loss-value based on a plurality of loss criteria, wherein the loss function may include: a first loss criteria based on an immunological response given two amino acid sequences; a second loss criteria that modifies the loss-value for sub-sequences not found in a dataset of wildtype sequences or sub-sequences not predicted to fold correctly; and a third loss criteria that, for each weight-vector, modifies the loss-value based on the greatest value in the second continuous values. Implementations of the described techniques may include hardware, a method or process, or computer software on a computer-accessible medium.

One general aspect includes a non-transitory, computer readable media storing instructions that, when executed by one or more processors, cause the one or more processors to perform operations that may include: receiving a discrete-data object comprising a plurality of first discrete values, the discrete-data object comprising one or more amino acid sequences; converting the discrete-data object into a continuous-data object comprising a plurality of first continuous values; applying, to the continuous-data object, a continuous-data algorithm to generate a continuous-result object comprising a plurality of second continuous values; converting the continuous-result object into a discrete-result object comprising a plurality of second discrete values; and manufacturing a vaccine comprising at least one of i) a protein defined by the discrete-result object, ii) a nucleic acid capable of producing the protein defined by the discrete-result object, and iii) a delivery vehicle capable of producing the protein defined by the discrete-result object. Other embodiments of this aspect include corresponding computer systems, apparatus, and computer programs recorded on one or more computer storage devices, each configured to perform the actions of the methods.

Implementations may include one or more of the following features. The media where the one or more amino acid sequences may include: a first amino acid sequence and a second amino acid sequence, each of the first and the second amino acid sequences including respective single letters or respective letter strings. Converting the discrete-data object into the continuous-data object may include: generating, for each first discrete value, a weight-vector of weight values, each weight value representing a likelihood that the first discrete value represents a particular amino acid; generating, for each weight value of each weight-vector, a property-vector of property values, each property value representing a physiochemical property of a particular amino acid; and combining the weight-vector and the property-vector to create the first continuous values of the continuous-data object. Each weight-vector has twenty weight values, each weight value corresponding to one of twenty possible amino acids. Converting the continuous-result object into the discrete-result object may include determining, for each second continuous value, a respective single amino acid, where the determined single amino acids form the plurality of second discrete values. Implementations of the described techniques may include hardware, a method or process, or computer software on a computer-accessible medium.

In certain embodiments, disclosed herein is an algorithm that can generate influenza antigens for use as a vaccine. In one implementation, this can include: 1) Generating a reduced-dimension space for all wildtype hemagglutinin sequences through machine learning (e.g., variational autoencoder architecture) using two steps:
a) Embedding variably into a reduced space, e.g., a model predicts mean and variance from input sequence, with embedded coordinates selected from normal distribution with predicted mean and variance; and
b) Decoding back to original sequence from reduced space location “autoencoder” loss function is then performed, reducing by the similarity of the input and output sequences.

2) Training an immune response prediction model based on location of antigen (vaccine candidate) and readout strains (target sequences) in the reduced dimensional space [input: antigen and readout embedded by model of step 1, output: measure of immune response such as antibody titer].

3) Sampling candidate vaccine component representations from the reduced space, ranking candidate vaccine component representations by predicted performance against target sequences using the model described in step 2, and identifying top candidates.

4) Decoding top candidate representations [using model from step 1b] to emit hemagglutinin sequences that may or may not have been observed in the original wildtype set.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a dimension-reducing method for generating amino acid sequences, the method being performed by a system of one or more computers. The method includes receiving one or more data objects defining a plurality of wild-type amino acid sequences. The method also includes generating, from the one or more data objects, a plurality of reduced-dimension sequences in a reduced-dimension space, where: each reduced-dimension sequence contains data respective of at least one of the wild-type amino acid sequences, the reduced-dimension space is of a lower dimensionality than the wild-type amino acid sequences, and the plurality of reduced-dimension sequences define a distribution of values along each dimension of the reduced-dimension space. The method also includes generating a plurality of candidate sequences in the reduced-dimension space using the plurality of reduced-dimension sequences. The method also includes receiving one or more data objects defining a viral amino acid sequence. The method also includes generating at least one reduced-dimension viral sequences in the reduced-dimension space. The method also includes providing, as input to a titer-predictor, each of the candidate sequences and at least one of the reduced-dimension viral sequences. The method also includes receiving, as output from the titer-predictor, a candidate-score for each of the candidate sequences. The method also includes selecting at least one candidate sequence from among the candidate sequences. The method also includes generating at least one new amino acid sequence for each of the selected candidate sequences. The method also includes providing the generated at least one amino acid sequence. The method also includes operations where each of the generated amino acid sequences is suitable for manufacturing a respective vaccine may include at least one of i) a protein defined by the generated amino acid sequence, ii) a nucleic acid capable of producing the protein defined by the generated amino acid sequence, and iii) a delivery vehicle capable of producing the protein defined by the generated amino acid sequence. Other embodiments of this aspect include corresponding computer systems, apparatus, and computer programs recorded on one or more computer storage devices, each configured to perform the actions of the methods.

Implementations may include one or more of the following features. The method includes operations where generating a plurality of reduced-dimension sequences may include creation of representations of the wild-type amino acid sequences using a variational autoencoder that predicts mean and variance values of input data. Each of the reduced-dimension sequences includes a respective group of values, and generating the plurality of candidate sequences in the reduced-dimension space may include sampling distributions of values of the plurality of reduced-dimension sequences. The titer-predictor is configured to: receive, as input, i) a first sequence in the reduced-dimension space and ii) a second sequence in the reduced-dimension space; and provide, as output, a titer-score as the candidate score, the titer-score defines a measure of biological response between the first sequence and the second sequence. Selecting the at least one candidate sequence as a selected candidate sequence may include selecting n candidate sequences with the highest candidate-scores. The method includes operations where n is a value of 1, such that a single candidate sequence is selected. The method includes operations where n is a value greater than 1, such that a plurality of candidate sequences are selected. Selecting the at least one candidate sequence as a selected candidate sequence may include selecting candidate sequences with respective candidate-scores greater than a threshold value. Each of the generated amino acid sequences is different from any of the wild-type amino acid sequences. At least one of the candidate sequences is in the plurality of reduced-dimension sequences. Implementations of the described techniques may include hardware, a method or process, or computer software on a computer-accessible medium.

One general aspect includes a system for generating amino acid sequences, the system may include computer memory. The system also includes one or more processors. The system also includes computer-memory storing instructions that, when executed by the processors, cause the processors to perform operations that may include: receiving one or more data objects defining a plurality of wild-type amino acid sequences; generating, from the one or more data objects, a plurality of reduced-dimension sequences in a reduced-dimension space, wherein: each reduced-dimension sequence contains data respective of at least one of the wild-type amino acid sequences, the reduced-dimension space is of a lower dimensionality than the wild-type amino acid sequences, and the plurality of reduced-dimension sequences define a distribution of values along each dimension of the reduced-dimension space, generating a plurality of candidate sequences in the reduced-dimension space using the plurality of reduced-dimension sequences; receiving one or more data objects defining a viral amino acid sequence; generating at least one reduced-dimension viral sequences in the reduced-dimension space; providing, as input to a titer-predictor, each of the candidate sequences and at least one of the reduced-dimension viral sequences; receiving, as output from the titer-predictor, a candidate-score for each of the candidate sequences; selecting at least one candidate sequence from among the candidate sequences; generating at least one new amino acid sequence for each of the selected candidate sequences; and providing the generated at least one amino acid sequence, wherein each of the generated amino acid sequences is suitable for manufacturing a respective vaccine comprising at least one of i) a protein defined by the generated amino acid sequence, ii) a nucleic acid capable of producing the protein defined by the generated amino acid sequence, and iii) a delivery vehicle capable of producing the protein defined by the generated amino acid sequence. Other embodiments of this aspect include corresponding computer systems, apparatus, and computer programs recorded on one or more computer storage devices, each configured to perform the actions of the methods.

Implementations may include one or more of the following features. The system where generating a plurality of reduced-dimension sequences may include creation of representations of the wild-type amino acid sequences using a variational autoencoder that predicts mean and variance values of input data. Each of the reduced-dimension sequences includes a respective group of values, and generating the plurality of candidate sequences in the reduced-dimension space may include sampling distributions of values of the plurality of reduced-dimension sequences. The titer-predictor is configured to: receive, as input, i) a first sequence in the reduced-dimension space and ii) a second sequence in the reduced-dimension space; and provide, as output, a titer-score as the candidate score, the titer-score defines a measure of biological response between the first sequence and the second sequence. Selecting the at least one candidate sequence as a selected candidate sequence may include selecting n candidate sequences with the highest candidate-scores. Implementations of the described techniques may include hardware, a method or process, or computer software on a computer-accessible medium.

One general aspect includes a non-transitory, computer readable media storing instructions that, when executed by one or more processors, cause the one or more processors to perform operations including: receiving one or more data objects defining a plurality of wild-type amino acid sequences; generating, from the one or more data objects, a plurality of reduced-dimension sequences in a reduced-dimension space, wherein: each reduced-dimension sequence contains data respective of at least one of the wild-type amino acid sequences, the reduced-dimension space is of a lower dimensionality than the wild-type amino acid sequences, and the plurality of reduced-dimension sequences define a distribution of values along each dimension of the reduced-dimension space, generating a plurality of candidate sequences in the reduced-dimension space using the plurality of reduced-dimension sequences; receiving one or more data objects defining a viral amino acid sequence; generating at least one reduced-dimension viral sequences in the reduced-dimension space; providing, as input to a titer-predictor, each of the candidate sequences and at least one of the reduced-dimension viral sequences; receiving, as output from the titer-predictor, a candidate-score for each of the candidate sequences; selecting at least one candidate sequence from among the candidate sequences; generating at least one new amino acid sequence for each of the selected candidate sequences; and providing the generated at least one amino acid sequence, wherein each of the generated amino acid sequences is suitable for manufacturing a respective vaccine comprising at least one of i) a protein defined by the generated amino acid sequence, ii) a nucleic acid capable of producing the protein defined by the generated amino acid sequence, and iii) a delivery vehicle capable of producing the protein defined by the generated amino acid sequence. Other embodiments of this aspect include corresponding computer systems, apparatus, and computer programs recorded on one or more computer storage devices, each configured to perform the actions of the methods.

Implementations may include one or more of the following features. The media where generating a plurality of reduced-dimension sequences may include creation of representations of the wild-type amino acid sequences using a variational autoencoder that predicts mean and variance values of input data. Each of the reduced-dimension sequences includes a respective group of values, and generating the plurality of candidate sequences in the reduced-dimension space may include sampling distributions of values of the plurality of reduced-dimension sequences. The titer-predictor is configured to: receive, as input, i) a first sequence in the reduced-dimension space and ii) a second sequence in the reduced-dimension space; and provide, as output, a titer-score as the candidate score, the titer-score defines a measure of biological response between the first sequence and the second sequence. Implementations of the described techniques may include hardware, a method or process, or computer software on a computer-accessible medium.

These and other aspects, features, and implementations can be expressed as methods, apparatus, systems, components, program products, methods of doing business, means or steps for performing a function, and in other ways, and will become apparent from the following descriptions, including the claims.

Implementations of the present disclosure can provide the following advantages. When compared with traditional techniques, vaccines can be designed for a future pathogenic season to confer more protection in terms of an amount of biological response for at least one pathogenic strain of that future pathogenic season. When compared with traditional techniques, vaccines can be designed for future pathogenic seasons to confer more protection in terms of breadth of effective coverage for a plurality of pathogenic strains of that future pathogenic season (that is, elicit an effective immunological response for a number of pathogenic strains in a future pathogenic season). Unlike traditional techniques, rarely observed strains that may confer “more protection” because they cross-react with more strains than frequently.

### Methods of Use

The present disclosure provides methods of administering the vaccine described herein to a subject. The methods may be used to vaccinate a subject against an influenza virus. In some embodiments, the vaccination method comprises administering to a subject in need thereof a vaccine comprising one or more recombinant influenza virus HAs as described herein, one or more recombinant influenza virus NAs as described herein, and an optional adjuvant in an amount effective to vaccinate the subject against influenza virus. Likewise, the present disclosure provides a vaccine comprising one or more influenza virus HAs as described herein, one or more NAs as described herein, and an optional adjuvant, for use in vaccinating a subject against an influenza virus. Also disclosed herein is an immunogenic composition comprising one or more recombinant influenza virus HAs as described herein, one or more recombinant influenza virus NAs as described herein, and an optional adjuvant, for the manufacture of a vaccine for use in vaccinating a subject against influenza virus.

The present disclosure also provides methods of immunizing a subject against influenza virus, comprising administering to the subject an immunologically effective amount of a vaccine comprising one or more recombinant influenza virus HAs as described herein, one or more recombinant influenza virus NAs as described herein, and an optional adjuvant. Likewise, the present disclosure provides a vaccine comprising one or more recombinant influenza virus HAs as described herein, one or more recombinant influenza virus NAs as described herein, and an optional adjuvant, for use in immunizing a subject against an influenza virus. Also disclosed herein is an immunogenic composition comprising one or more recombinant influenza virus HAs as described herein, one or more recombinant influenza virus NAs as described herein, and an optional adjuvant, for the manufacture of a vaccine for use in immunizing a subject against influenza virus.

In some embodiments, the method or use prevents influenza virus infection or disease in the subject. In some embodiments, the method or use raises a protective immune response in the subject. In some embodiments, the protective immune response is an antibody response.

The methods of immunizing (or related uses) provided herein can elicit a broadly neutralizing immune response against one or more influenza viruses. Accordingly, in various embodiments, the composition described herein can offer broad cross-protection against different types of influenza viruses. In some embodiments, the composition offers cross-protection against avian, swine, seasonal, and/or pandemic influenza viruses. In some embodiments, the methods of immunizing (or related uses) are capable of eliciting an improved immune response against one or more seasonal influenza strains (e.g., a standard of care strain). For example, the improved immune response may be an improved humoral immune response. In some embodiments, the methods of immunizing (or related uses) are capable of eliciting an improved immune response against one or more pandemic influenza strains. In some embodiments, the methods of immunizing (or related uses) are capable of eliciting an improved immune response against one or more swine influenza strains. In some embodiments, the methods of immunizing (or related uses) are capable of eliciting an improved immune response against one or more avian influenza strains.

In certain embodiments, provided herein are methods of enhancing or broadening a protective immune response in a subject, the method comprising administering to the subject an immunologically effective amount of the vaccine disclosed herein. Likewise, the present disclosure provides any of the vaccine s described herein for use in enhancing or broadening a protective immune response in a subject, including, for example, a vaccine comprising a plurality of recombinant influenza virus proteins, wherein the plurality of recombinant influenza virus proteins comprises or consists of one or more recombinant influenza virus HA and one or more recombinant influenza virus NA. Also disclosed herein is an immunogenic composition as described herein for the manufacture of a vaccine for use in enhancing or broadening a protective immune response in a subject. In certain embodiments, the vaccine disclosed herein increases the vaccine efficacy of a standard of care influenza virus vaccine composition by an amount ranging from about 5% to about 100%, such as from about 10% to about 25%, from about 20% to about 100%, from about 15% to about 75%, from about 15% to about 50%, from about 20% to about 75%, from about 20% to about 50%, or from about 40% to about 80%, such as about 40% to about 60% or about 60% to about 80%. In certain embodiments, the vaccine disclosed herein has a vaccine efficacy that is at least 5% greater than the vaccine efficacy of a standard of care influenza virus vaccine, such as a vaccine efficacy that is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% greater than the vaccine efficacy of a standard of care influenza virus vaccine.

In certain embodiments, the standard of care influenza virus vaccine may be an inactivated influenza vaccine (IIV), such as a trivalent or a quadrivalent IIV. Typically, the standard of care, inactivated influenza virus vaccine composition comprises inactivated influenza virus from an H1N1 strain, an H3N2 strain, a B/Victoria lineage, and a B/Yamagata lineage. In certain embodiments, the standard of care influenza virus vaccine may comprise recombinant influenza virus HA, such as a trivalent or a quadrivalent vaccine composition comprising recombinant influenza virus HA. Typically, the standard of care, recombinant HA vaccine composition comprises rHA from an H1N1 strain, an H3N2 strain, a B/Victoria lineage, and a B/Yamagata lineage. Vaccine efficacy may be expressed as a proportion of reduction in disease between a vaccinated population and an unvaccinated population or a population administered a different vaccine. In certain embodiments, vaccine efficacy can be calculated by subtracting the rate of disease cases in a vaccinated population from the rate of disease cases in an unvaccinated population, and dividing by the rate of disease cases in the unvaccinated population according to the following formula: [(Rate of disease in an unvaccinated population)−(Rate of disease in a vaccinated population)/(Rate of disease in an unvaccinated population)×100].

Also provided are methods of preventing influenza virus disease in a subject, comprising administering to the subject a vaccine comprising one or more recombinant influenza virus HAs as described herein, one or more recombinant influenza virus NAs as described herein, and an optional adjuvant in an amount effective to prevent influenza virus disease in the subject. Likewise, the present disclosure provides a vaccine comprising one or more recombinant influenza virus HAs as described herein, one or more recombinant influenza virus as described herein, and an optional adjuvant, for use in preventing influenza virus disease in a subject. Also disclosed herein is an immunogenic composition comprising one or more recombinant influenza virus HAs as described herein, one or more recombinant influenza virus NAs as described herein, and an optional adjuvant, for the manufacture of a vaccine for use in preventing influenza virus disease in a subject.

Also provided are methods of inducing an immune response against an influenza virus HA and an influenza virus NA in a subject, comprising administering to the subject a vaccine comprising one or more recombinant influenza virus HAs as described herein, one or more recombinant influenza virus NAs as described herein, and an optional adjuvant. Likewise, the present disclosure provides a vaccine comprising one or more recombinant influenza virus HAs as described herein, one or more recombinant influenza virus NAs as described herein, and an optional adjuvant, for use in inducing an immune response against an influenza virus HA and an influenza virus NA in a subject. Also disclosed herein is an immunogenic composition comprising one or more recombinant influenza virus HAs as described herein, one or more recombinant influenza virus NAs as described herein, and an optional adjuvant, for the manufacture of a vaccine for use in inducing an immune response against an influenza virus HA and an influenza virus NA in a subject.

Vaccines comprising one or more recombinant influenza virus HAs as described herein, one or more recombinant influenza virus NAs as described herein, and an optional adjuvant may be administered prior to or after development of one or more symptoms of an influenza infection. That is, in some embodiments, the vaccine described herein may be administered prophylactically to prevent influenza infection or ameliorate the symptoms of a potential influenza infection. In some embodiments, a subject is at risk of influenza virus infection if the subject will be in contact with other individuals or livestock (e.g., swine) known or suspected to have been infected with seasonal or pandemic influenza virus and/or if the subject will be present in a location in which influenza infection is known or thought to be prevalent or endemic. In some embodiments, the vaccine is administered to a subject suffering from an influenza infection, or the subject is displaying one or more symptoms commonly associated with influenza infection. In some embodiments, the subject is known or believed to have been exposed to an influenza virus. In some embodiments, a subject is at risk or susceptible to an influenza infection if the subject is known or believed to have been exposed to the influenza virus. In some embodiments, a subject is known or believed to have been exposed to the influenza virus if the subject has been in contact with other individuals or livestock (e.g., swine) known or suspected to have been infected with pandemic influenza virus and/or if the subject is or has been present in a location in which influenza infection is known or thought to be prevalent or endemic. The vaccine disclosed herein may be used to treat or prevent disease caused by either or both a seasonal and a pandemic influenza strain.

Vaccines in accordance with the disclosure may be administered in any amount or dose appropriate to achieve a desired outcome. In some embodiments, the desired outcome is induction of a lasting adaptive immune response against a broad spectrum of influenza strains, including both seasonal and pandemic strains. In some embodiments, the desired outcome is reduction in intensity, severity, and/or frequency, and/or delay of onset of one or more symptoms of influenza infection. The dose required may vary from subject to subject depending on the species, age, weight and general condition of the subject, the severity of the infection being treated, the particular composition being used and its mode of administration.

In various embodiments, the vaccine or immunogenic compositions described herein are administered to subjects, wherein the subjects can be any member of the animal kingdom. In some embodiments, the subject is a non-human animal. In some embodiments, the non-human subject is an avian (e.g., a chicken or a bird), a reptile, an amphibian, a fish, an insect, and/or a worm. In some embodiments, the non-human subject is a mammal (e.g., a ferret, a rodent, a mouse, a rat, a rabbit, a monkey, a dog, a cat, a sheep, cattle, a primate, and/or a pig).

In some embodiments, the vaccine or immunogenic compositions described herein are administered to a human subject. In particular embodiments, a human subject is 6 months of age or older, 6 months through 35 months of age, at least two years of age, at least 3 years of age, 36 months through 8 years of age, 9 years of age or older, at least 6 months of age and less than 5 years of age, at least 6 months of age and less than 18 years of age, or at least 3 years of age and less than 18 years of age. In some embodiments, the human subject is an infant (less than 36 months). In some embodiments, the human subject is a child or adolescent (less than 18 years of age). In some embodiments, the human subject is a child of at least 6 months of age and less than 5 years of age. In some embodiments, the human subject is at least 5 years of age and less than 60 years of age. In some embodiments, the human subject is at least 5 years of age and less than 65 years of age. In some embodiments, the human subject is elderly (at least 60 years of age or at least 65 years of age). In some embodiments, the human subject is a non-elderly adult (at least 18 years of age and less than 65 years of age or at least 18 years of age and less than 60 years of age).

Typically, the methods and uses of the vaccines described herein include administration of a single dose to a subject (i.e., no booster dose). However, in some embodiments, the methods and uses of the vaccines described herein include prime-boost vaccination strategies. Prime-boost vaccination comprises administering a priming vaccine and then, after a period of time has passed, administering to the subject a boosting vaccine. The immune response is “primed” upon administration of the priming vaccine and is “boosted” upon administration of the boosting vaccine. The priming vaccine can include a vaccine comprising the one or more recombinant influenza virus HAs, the one or more recombinant influenza virus NAs, and an optional adjuvant. Likewise, the boosting vaccine can include a vaccine comprising the one or more recombinant influenza virus HAs, the one or more recombinant influenza virus NAs, and an optional adjuvant. The priming vaccine can be, but need not be, the same as the boosting vaccine. Administration of the boosting vaccine is generally weeks or months after administration of the priming composition, preferably about 2-3 weeks or 4 weeks, or 8 weeks, or 16 weeks, or 20 weeks, or 24 weeks, or 28 weeks, or 32 weeks. In certain embodiments, the recipient of the prime-boost vaccination is a naïve subject, typically a naïve infant or child.

The vaccine can be administered using any suitable route of administration, including, for example, parenteral delivery, as discussed above.

Typically, the one or more recombinant influenza virus HAs as described herein, the one or more recombinant influenza virus NAs as described herein, and the optional adjuvant are administered together as components of the same vaccine. However, it is not necessary for the one or more recombinant influenza virus HAs, the one or more recombinant influenza virus NAs, and/or the optional adjuvant to be administered as part of the same vaccine. That is, if desired, the one or more recombinant influenza virus HAs, the one or more recombinant influenza virus NAs, and/or the optional adjuvant can be administered to the subject separately. For example, a first vaccine comprising the one or more recombinant influenza virus HAs, may be administered to a subject separately from a second vaccine comprising the one or more recombinant influenza virus NAs. When the first and second vaccines are administered separately, the first and second vaccines may be administered to the subject at different sites.

### Representative Embodiments of the Disclosure

1. An immunogenic composition comprising a plurality of recombinant influenza virus proteins, wherein the plurality of recombinant influenza virus proteins comprises:
a first recombinant influenza virus hemagglutinin (HA), wherein the first recombinant influenza virus HA is an H1 HA;
a second recombinant influenza virus HA, wherein the second recombinant influenza virus HA is an H3 HA;
a third recombinant influenza virus HA, wherein the third recombinant influenza virus HA is from a B/Victoria lineage;
a fourth recombinant influenza virus HA, wherein the fourth recombinant influenza virus HA is from a B/Yamagata lineage;
a first recombinant influenza virus neuraminidase (NA), wherein the first recombinant influenza virus NA is an N1 NA;
a second recombinant influenza virus NA, wherein the second recombinant influenza virus NA is an N2 NA;
a third recombinant influenza virus NA, wherein the third recombinant influenza virus NA is from a B/Victoria lineage; and
   a fourth recombinant influenza virus NA, wherein the fourth recombinant influenza virus NA is from a B/Yamagata lineage.

2. The immunogenic composition of embodiment 1, wherein each of the first, second, third, and fourth recombinant influenza virus NA is a modified recombinant influenza virus NA.

3. The immunogenic composition of embodiment 2, wherein the modified recombinant influenza virus NA comprises a modified recombinant tetrameric influenza virus NA comprising four modified monomeric NA molecules, each comprising a head region of the NA of the influenza virus, but lacking a cytoplasmic tail, a transmembrane region, and all or substantially all of a stalk region of the NA of the influenza virus and wherein the modified monomeric NA molecules form modified recombinant tetrameric NA when expressed in a host cell.

4. The immunogenic composition of embodiment 3, wherein each modified recombinant monomeric influenza virus NA comprises a heterologous tetramerization domain.

5. The immunogenic composition of embodiment 3, wherein each modified recombinant monomeric influenza virus NA does not comprise a heterologous oligomerization domain.

6. The immunogenic composition of embodiment 4, wherein the heterologous tetramerization domain is a Staphylothermus marinus tetrabrachion tetramerization domain, a GCN4 leucine zipper tetramerization domain, a tetramerization domain from a paramyxovirus phosphoprotein, or a human vasodilator stimulated phosphoprotein (VASP) tetramerization domain.

7. The immunogenic composition according to any of the preceding embodiments, wherein each of the recombinant influenza virus HA is produced by a baculovirus expression system in cultured insect cells.

8. The immunogenic composition according to any of the preceding embodiments, wherein each of the recombinant influenza virus NA is produced in Chinese Hamster Ovary (CHO) cells.

9. The immunogenic composition according to any of the preceding embodiments, wherein the immunogenic composition does not contain inactivated influenza virions or live attenuated influenza virions.

10. The immunogenic composition according to any of the preceding claims, wherein each of the recombinant influenza virus HAs and/or each of the recombinant influenza virus NAs are from standard of care influenza strains.

11. The immunogenic composition according to any of the preceding embodiments, wherein the H1 HA is from an H1N1 influenza virus strain and/or the H3 HA is from an H3N2 influenza virus strain.

12. The immunogenic composition according to any of the preceding embodiments, wherein the N1 NA is from an H1N1 influenza virus strain and/or the N2 NA is from an H3N2 influenza virus strain.

13. The immunogenic composition according to any of the preceding embodiments, wherein the H1 HA is from an H1N1 influenza virus strain, the H3 HA is from an H3N2 influenza virus strain, the N1 NA is from an H1N1 influenza virus strain, and the N2 NA is from an H3N2 influenza virus strain.

14. The immunogenic composition of embodiment 13, wherein the H1 HA and the N1 NA are from the same H1N1 influenza virus strain and the H3 HA and N2 NA are from the same H3N2 influenza virus strain.

15. The immunogenic composition according to any of the preceding embodiments, wherein the plurality of recombinant influenza virus proteins consists of:
a first recombinant influenza virus HA, wherein the first recombinant influenza virus HA is an H1 HA;
a second recombinant influenza virus HA, wherein the second recombinant influenza virus HA is an H3 HA;
a third recombinant influenza virus HA, wherein the third recombinant influenza virus HA is from a B/Victoria lineage;
a fourth recombinant influenza virus HA, wherein the fourth recombinant influenza virus HA is from a B/Yamagata lineage;
a first recombinant influenza virus NA, wherein the first recombinant influenza virus NA is an N1 NA;
a second recombinant influenza virus NA, wherein the second recombinant influenza virus NA is an N2 NA;
a third recombinant influenza virus NA, wherein the third recombinant influenza virus NA is from a B/Victoria lineage; and
a fourth recombinant influenza virus NA, wherein the fourth recombinant influenza virus NA is from a B/Yamagata lineage.

16. The immunogenic composition according to any of the preceding embodiments, wherein the composition further comprises an adjuvant.

17. The immunogenic composition according to embodiment 16, wherein the adjuvant comprises a squalene-in-water adjuvant or a liposome-based adjuvant.

18. The immunogenic composition according to embodiment 17, wherein the squalene-in-water adjuvant comprises AF03.

19. The immunogenic composition according to embodiment 17, wherein the liposome-based adjuvant comprises SPA14.

20. The immunogenic composition according to any of the preceding embodiments, wherein each of the recombinant influenza virus HAs is present in the composition in an amount ranging from about 0.1 μg to about 90 μg, optionally about 1 μg to about 60 μg or 5 μg to about 45 μg.

21. The immunogenic composition according to any of the preceding embodiments, wherein each of the recombinant influenza virus NAs is present in the composition in an amount ranging from about 0.1 μg to about 90 μg, optionally about 1 μg to about 60 μg or about 5 μg to about 45 μg.

22. The immunogenic composition according to any of the preceding embodiments, wherein the composition is formulated for intramuscular injection.

23. A vaccine comprising the immunogenic composition according to any of the preceding claims and a pharmaceutical carrier.

24. A method of immunizing a subject against influenza virus, the method comprising administering to the subject an immunologically effective amount of the vaccine of claim 23.

25. The method of embodiment 24, wherein the method prevents influenza virus infection in the subject.

26. The method of embodiments 24 or 25, wherein the method raises a protective immune response in the subject.

27. The method of embodiment 26, wherein the protective immune response comprises an HA antibody response and/or an NA antibody response.

28. The method of any one of embodiments 24-27, wherein the subject is human.

29. The method of any one of embodiments 24-28, wherein the vaccine is administered intramuscularly, intradermally, subcutaneously, intravenously, intranasally, by inhalation, or intraperitoneally.

30. The method of any one of embodiments 24-29, wherein the method treats or prevents disease caused by either or both a seasonal and a pandemic influenza strain.

31. The method of any one of embodiments 24-30, wherein the subject is human and the human is 6 months of age or older, less than 18 years of age, at least 6 months of age and less than 18 years of age, at least 18 years of age and less than 65 years of age, at least 6 months of age and less than 5 years of age, at least 5 years of age and less than 65 years of age, at least 60 years of age, or at least 65 years of age.

32. A method of reducing one or more symptoms of influenza virus infection, the method comprising administering to a subject a prophylactically effective amount of the vaccine of embodiment 23.

33. A method of enhancing or broadening a protective immune response in a subject, the method comprising administering to the subject an immunologically effective amount of the vaccine of embodiment 23, wherein the vaccine increases the vaccine efficacy of a standard of care influenza virus vaccine composition by an amount ranging from about 5% to about 100%, such as at least about 20%, or from about 40% to about 80%, such as from about 40% to about 60%.

34. The method according to embodiment 33, wherein the standard of care influenza virus vaccine composition is an inactivated influenza virus composition comprising inactivated influenza virus from an H1N1 strain, an H3N2 strain, a B/Victoria lineage, and a B/Yamagata lineage.

35. The method according to embodiment 33, wherein the standard of care influenza virus vaccine comprises recombinant influenza virus HA from an H1N1 strain, an H3N2 strain, a B/Victoria lineage, and a B/Yamagata lineage.

36. The method of any one of embodiments 24-35, comprising administering to the subject two doses of the vaccine with an interval of 2-6 weeks, optionally 4 weeks.

The present disclosure will be more fully understood by reference to the following Examples.

### Examples

The following examples are to be considered illustrative and not limiting on the scope of the disclosure described above.

Animal experiments were carried out in compliance with the Public Health Service (PHS) Policy on Humane Care and Use of Laboratory Animals and the Guide for the Care and Use of Laboratory Animals, and were conducted with approved animal protocols from the Sanofi Institutional Animal Care and Use Committee (IACUC). All animals were housed under specified pathogen-free conditions with food and water ad libitum.

Influenza viruses: Reassortant H6 viruses used in enzyme-linked lectin assay (ELLA) were generated by reverse genetics, with each reassortant expressing the targeted NA antigen, the HA from A/mallard/Sweden/81/2002 H6N1, and internal genes from A/Puerto Rico/8/1934 H1N1 (“PR8”). HA and NA segments including non-coding regions were generated by custom gene synthesis (Geneart AG), and PR8 segments were derived from viral isolates. All segments were cloned into a bi-directional transcription plasmid derived from pUC57 (Genscript) through the incorporation of polymerase (Pol) I and Pol II promoters. Briefly, 293FT cells (Thermo Fisher Scientific) were transfected with a total of eight plasmids representing each influenza virus segment using Lipofectamine 2000 CD (Thermo Fisher Scientific). After 24 hours, MDCK-ATL cells (ATCC) were added to the transfected cells in the presence of TPCK-treated trypsin (Sigma) to allow influenza virus propagation. Cell culture supernatants containing influenza virus were harvested 7 days post-MDCK addition and passaged in 8-to-10-day-old embryonated chicken eggs (Charles River Laboratories, Inc.). Inoculated eggs were incubated at 37° C. for 48 h, then cooled to 4° C. for 12 h, harvested, and clarified by low-speed centrifugation (3,000 rpm, 20 min). Virus titers were determined by plaque assay on MDCK cells.

Egg-grown stocks of A/Michigan/45/2015 (H1N1), A/Singapore/INFIMH-16-0019/2016 (H3N2), B/Colorado/06/2017 (Victoria lineage), B/Maryland/15/2016 (Victoria lineage), and B/Phuket/3073/2013 (Yamagata lineage) included in HAI testing were provided by Sanofi Paster Global Clinical Immunology (Swiftwater, PA). Wild-type influenza A/Perth/16/2009 (H3N2) used in ferret studies was provided by IIT Research Institute (Chicago, IL). All viruses were stored below −65° C. until use.

Vaccine antigens: Constructs were designed for the expression of recombinant, soluble influenza NA. Both tetrameric and monomeric NA construct design includes an N-terminal CD5 secretion signal peptide, an optional 6HIS tag (for purification) and the globular neuraminidase head domain. The tetrameric design (rTET-NA) also contains a tetrabrachion domain between the HIS tag and the globular head for multimerization. Using a defined amino acid sequence, a codon optimized synthetic gene was assembled from oligonucleotides and/or PCR products and the fragment was inserted into pcDNA3.4-TOPO (ThermoFisher). The plasmid DNA was purified from transformed bacteria and scaled to achieve appropriate concentration for transfection. Protein expression was performed in CHO-S cells using the ExpiCHO™ Expression System Max Titer Protocol (ThermoFisher). A clarification step was performed to separate secreted proteins from cells. NA protein was purified from host cell proteins by affinity (HisTrap™ HP Column—GE Healthcare) followed by anion exchange chromatography (HiTrap™ Q HP—GE HealthCare), dialysis into 10 mM phosphate buffered saline (pH 7.2) and a 0.2 m sterile filtration. The NA vaccine preparations were produced in compliance with the current good research practices (cGRP).

Enzyme-Linked Lectin Assay (ELLA) Assessment of NAI Responses: NAI antibody responses were measured against H6 reassortant viruses containing NA derived from strains of interest by ELLA as previous described in Couzens, An optimized enzyme-linked lectin assay to measure influenza A virus neuraminidase inhibition antibody titers in human sera, J. VIROLOGICAL METHODS 2014, 210:7-14. Briefly, a H6 reassortant virus containing the NA derived from a strain of interest was titrated in fetuin-coated 96-well plates to determine the standard amount of virus that provides 70% of maximum NA enzymatic activity. Titration of NAI antibodies present in the sera was achieved by performing two-fold serial dilutions of heat inactivated sera. A total of 50 μL of diluted sera was then added to 50 μL of diluted virus corresponding to 70% of maximum NA enzymatic activity in a fetuin-coated plate. The serum-virus mixture was incubated at 37° C. overnight. The plate was washed four times, incubated with horseradish peroxidase- (HRP-) conjugated peanut agglutinin (PNA) and washed again prior developing by addition of o-phenylenediamine dihydrochloride (OPD). Low or no signal relative to a virus control indicates inhibition of NA activity due to the presence of NA-specific antibodies. NAI titers were approximated with non-linear four parameter logistic (4PL) curve using GraphPad Prism software and the 50% maximal inhibitory concentration (IC₅₀) calculated.

Hemagglutinin-Inhibition (HAI) Assay: Sera were treated with receptor-destroying enzyme (RDE; Denka Seiken, Co., Japan) to inactivate nonspecific inhibitors prior to HAI assay. RDE-treated sera were serially diluted (2-fold dilutions) in v-bottom microtiter plates. An equal volume of each virus from the HAI readout panel was added to each well (4 hemagglutinating units (HAU) per well). For the present Examples, unless otherwise indicated the homologous virus panel included A/Michigan/45/2015 (H1N1), A/Singapore/INFIMH-16-0019/2016 (H3N2), B/Colorado/06/2017 or B/Maryland/15/2016 (Victoria lineage) and B/Phuket/3073/2013 (Yamagata lineage) viruses grown in eggs. The plates were covered and incubated at room temperature for 20 minutes (or 45 to 60 min), followed by the addition of 1% mixture of chicken erythrocytes (red blood cells; CRBC) or 0.5% mixture of turkey red blood cells (TRBC) (Lampire Biologicals) in PBS. The plates were mixed by agitation and covered, and the RBCs were allowed to settle for approximately 30 min to 1 hour at room temperature. The HAI titer was determined by the reciprocal dilution of the last well which contained non-agglutinated RBCs.

Antibody Forensics Assay: Antibody forensics methods (AFs) were used to measure strain-specific rHA antibodies in ferret sera using magnetic bead array (MagPlex® Microspheres) with fluorescent dyes. The strength of antibody binding to strain-specific rHA was presented in normalized mean fluorescent intensity units (nMFI), calculated from raw fluorescent intensity signal multiplied by the serum dilution. The rHAs coupled to the magnetic beads were selected based on antigenicity data published in the annual and interim reports on the composition of influenza vaccines by the Francis Crick Institute. In addition to 2018-2019 northern hemisphere recommended strains, rH3 panel included strains for 2013 through 2016 seasons, while H1 panel encompassed strains from 2009 through 2016 seasons. Individual ferret sera were analyzed and the resultant antibody forensics data for 40 H3 and 18 H1 strains was evaluated.

HINT mNT Influenza Protocol: Neutralization titers against influenza strains were measured as adapted from Jorquera, P. A. et al, Insights into the antigenic advancement of influenza A (H3N2) viruses, 2011-2018, Sci. Reports 9, 2676 (2019). Briefly, serial 2-fold dilutions of RDE treated sera from 1:20 to 1:2,560 were mixed with an equal volume of virus, about 1000 focus forming units (FFU), and incubated for 60 minutes at 37° C. After incubation, an MDCK-SIAT1 cell suspension was added to the virus:sera mixture and incubated for about 22 hrs. The monolayers were fixed with methanol and prepared for staining. Wells were then incubated with anti-influenza monoclonal antibody against nucleoprotein (NP), followed by an Alexa Fluor® 488-conjugated secondary antibody. Cells were washed and plates scanned on CTL ImmunoSpot® Cell Imaging v2. Counts from plate were transferred into Graphpad Prism software to calculate neutralization titers that achieves 50% foci reduction from sigmoidal curve. The assay does not include trypsin and measures inhibition of virus entry as compared to virus input control wells with no sera. The counts were individual infected cells, and the assay is suitable for all live virus subtypes, including H1, H3, BVic, and BYam.

The following rHAs were used in the antibody forensics assay disclosed herein: H3 AF panel: A/URUGUAY/716/2007, A/VICTORIA/361/2011, A/HONGKONG/4801/2014, A/SINGAPORE/INFIMH-16-0019/2016, A/BRISBANE/1059/2017, A/ETHIOPIA/1877/2017, A/KENYA/105/2017, A/MISSOURI/37/2017, A/MIYAZAKI/89/2017, A/OSORNO/60580/2017, A/SAPPORO/46/2017, A/SHANGHAIXUHUI/1373/2017, A/SYDNEY/1093/2017, A/SYDNEY/1013/2017, A/AKSARAY/4048/2016, A/ALBANIA/7165/2016, A/ANKARA/4110/2016, A/BRETAGNE/2836/2016, A/BRISBANE/1009/2016, A/CALIFORNIA/168/2016, A/CHIBA/33/2016, A/CHRISTCHURCH/513/2016, A/GUANGXIQIXIN/328/2016, A/HAWAII/67/2016, A/JORDAN/J16420301NT/2016, A/KAWASAKI/142/2016, A/KHMELNITSKY/719/2016, A/LAOS/F2884/2016, A/LINKOU/0051/2016, A/LISBOA/NIEVA063/2016, A/MARTINIQUE/531/2016, A/MARYLAND/24/2016, A/MEKNES/168/2016, A/MICHIGAN/84/2016, A/PORTUGAL/MS68/2016, A/SAUDIARABIA/192150/2016, A/SHANDONGLAICHENG/1763/2016, A/SINGAPORE/GP2366/2016, A/TASMANIA/97/2016, A/TOWNSVILLE/51/2016.

H1 AF panel: A/CALIFORNIA/07/2009, A/BAYERN/69/2009, A/HONGKONG/34079/2009, A/HONGKONG/33597/2009, A/LVIV/N6/2009, A/MONTPELLIER/2051/2009, A/CHRISTCHURCH/16/2010, A/ANKARA/TR40/2011, A/ASTRAKHAN/1/2011, A/HONGKONG/3934/2011, A/GOTEBORG/1/2011, A/MEXICO/2208/2011, A/HONG/KONG/5659/2012, A/STOCKHOLM/25/2012, A/ISRAEL/Q504/2015, A/MICHIGAN/45/2015, A/HUNGARY/12/2016, A/BRATISLAVA/342/2016.

For the Examples that follow, recombinant HA proteins were obtained from Protein Sciences. Briefly, purified HA proteins were produced in a continuous insect cell line (EXPRESSF+®) derived from Sf9 cells and grown in serum-free medium. IIV was prepared from influenza virus propagated in embryonated chicken eggs, inactivated with formaldehyde, concentrated, and purified by zonal centrifugation on a sucrose gradient, split with Triton® X-100, further purified and then suspended in sodium phosphate-buffered isotonic sodium chloride solution. Preparations were sterile filtered using 0.2 m syringe filter. Live influenza virus-derived neuraminidase (LVNA) was isolated from influenza virus propagated in embryonated chicken eggs. Virus was purified by sucrose gradient ultracentrifugation and NA was extracted by detergent solubilization, further purified by column chromatography, and suspended in sodium phosphate-buffered isotonic sodium chloride solution. Preparations were sterile filtered using 0.2 μm syringe filter.

### Example 1—Expression, Purification, and Characterization of rTET-NA

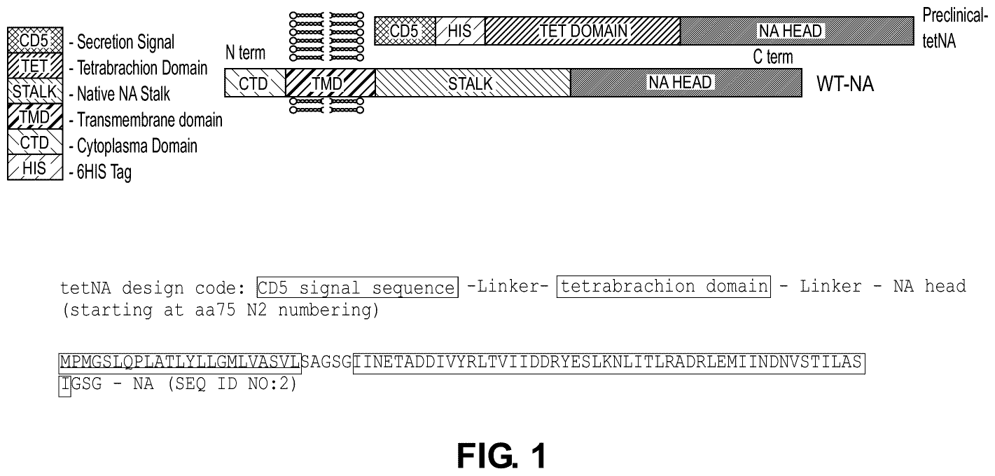

rTET-NA constructs derived from NAs across all four subtypes present in currently circulating seasonal influenza viruses (A/Michigan/45/2015 N1; A/Singapore/2016 N2; B/Colorado/06/2017 Victoria lineage; and B/Phuket/3073/2013 Yamagata lineage) were expressed in CHO-S cells and purified to near homogeneity for further characterization. A schematic representation and partial amino acid sequence of a rTET-NA construct (e.g. derived from A/Singapore/2016 N2) is shown in FIG. 1. Unless otherwise indicated, a heterologous tetrabrachion tetramerization sequence is present in all rTET-NA constructs disclosed in the following examples, independent of the influenza virus strain from which the NA head region was obtained. However, as known in the art, any suitable heterologous tetramerization domain can be used in place of the tetrabrachion tetramerization sequence. In the rTET-NA constructs, the cytoplasmic domain, the transmembrane region, and all or substantially all of the stalk region of the wild type influenza neuraminidase are replaced by a “secretion signal” peptide, a heterologous tetrabrachion tetramerization domain, and an optional histidine tag, which can be used to facilitate purification of the rTET-NA.

Efficient tetramerization of the rTET-NAs was demonstrated by size exclusion chromatography with multi-angle light scattering (SEC-MALS), and it was confirmed that all rTET-NAs were formed as tetramers, with a molecular mass (about 260 kDa) that was about four-fold greater than control (ectodomain) monomers (about 74 kDa) (data not shown). Size exclusion chromatography was undertaken with a TSK-GEL G4000 PWXL (7.8 mm×30 cm) column (Tosoh Bioscience) with a mobile phase of phosphate buffer saline containing 0.02% sodium azide (pH 7.4). All detectors were connected in line downstream of the column. Empower® (Waters Corporation, Milford, MA) software was used to integrate UV peak areas on the chromatogram. The purity of the sample was calculated based on the percentage of the specific peak area/the total peak areas. The molecular weight (MW) of protein in the peaks was determined by ASTRA (Wyatt Technologies, Santa Barbara, CA) software using light scattering signals with a concentration detector (RI or UV).

rTET-NA enzymatic activity was demonstrated with the 2′-(4-methylumbelliferyl)-α-d-N-acetylneuraminic acid (MUNANA) assay. Two-fold serial rTET-NA dilutions were prepared in 96-well plates using buffer (33.3 mM 2-[N-morpholino] ethanesulfonic acid [MES, pH 6.5], 4 mM CaCl₂), 50 mM BSA) and mixed with MUNANA substrate (100 μM) and incubate for 1 hour at 37° C. with shaking. The reaction was stopped by addition of alkaline solution (0.2M Na₂CO₃). The fluorescence intensity (RFU, relative fluorescence unit) from the rTET-NA and MUNANA substrate mixture was measured using excitation and emission wavelengths of 355 nm and 460 nm, respectively. A standard curve was generated using 4-methylumbelliferone (4-MU) diluted in enzyme buffer at various concentrations; rTET-NA enzymatic activity was determined against a 4MU reference with the results expressed in μM/60 min for total NA activity and nmole/min/μg for specific NA activity.

Additionally, rTET was shown to bind to oseltamivir-phosphate. In contrast, monomeric N2 ectodomain variants that were enzymatically inactive did not bind to oseltamivir-phosphate. SEC-MALS analysis was also performed on rTET-NA derived from different influenza subtypes and 34 different N2 strains, wherein tetramerization of the rTET-NAs and binding to oseltamivir-phosphate was demonstrated in all cases. The oseltamivir-binding assay was performed using the following conditions: capture an oseltamivir-phosphate-biotin conjugate (5-10 μg/ml in 1×KB buffer (1% BSA and 0.02% Tween in PBS) on the surface of streptavidin-coated biosensors; dip the biosensors into wells containing serial 2-fold dilutions of a sample of recombinant NA (0.16-10 μg/ml in 1×KB); and measure the binding kinetics of the recombinant NA to oseltamivir-phosphate using the bio-layer interferometry (BLI) technique on an Octet instrument (ForteBio, Molecular Devices, LLC).

### Example 2—Evaluation of rTET-NA Immunogenicity in Mice

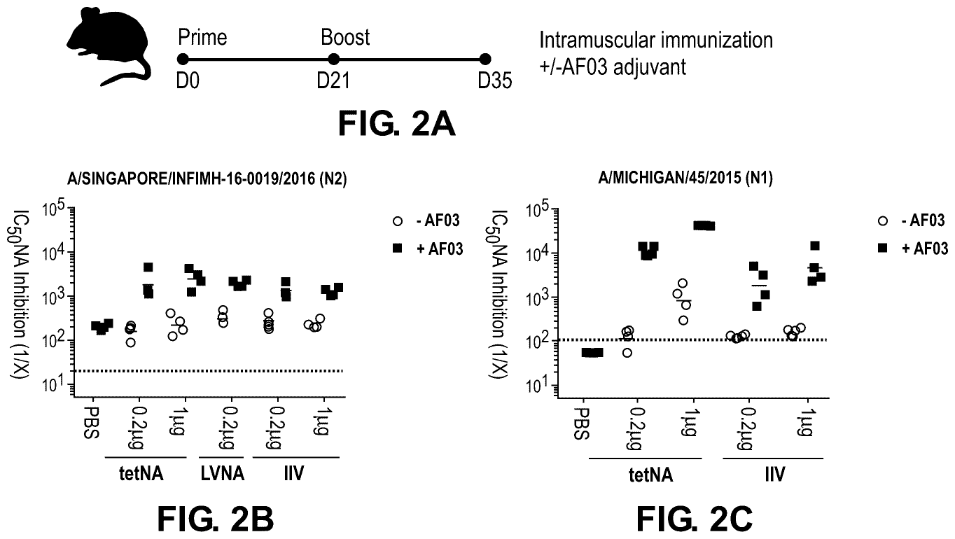

Mouse models were used to assess the immunogenicity of rTET-NA. Female BALB/c mice (8 per group) aged 6-8 weeks were vaccinated twice with either 0.2 μg or 1 μg of N2 rTET-NA derived from A/Singapore/INFIMH-16-0019/2016, N1 rTET-NA derived from A/Michigan/45/2015, monovalent inactivated influenza vaccine (IIV), or 0.2 μg live virus-derived NA (LVNA) with or without AF03 (squalene-in-water) adjuvant (all doses were 50 μL). As shown in FIG. 2A, the first dose was administered intramuscularly on Day 0, with a booster dose administered intramuscularly on Day 21. NAI antibody titers were measured in sera two weeks after the last dose (Day 35). Sera pools from two animals were created (stored at −20° C. until required), resulting in a total of 4 samples per group. The sera were tested by ELLA to assess NAI activity or via ELISA to derive NA-binding antibodies.

It was demonstrated that rTET-NA had comparable immunogenicity to other NA-containing viral preparations, which was markedly enhanced with AF03 adjuvant. See FIGS. 2B and 2C. As shown in FIG. 2C, the immunogenicity of the rTET-NA was higher than IIV for the N1 subtype, strain A/Michigan45/2015, which was similarly tested in mice. While not wishing to be bound by theory, it is possible that this subtype specific difference may be due to the split inactivation process for the later, resulting in reduced enzymatic activity and loss of immunogenicity as compared to N2.

### Example 3—Evaluation of rTET-NA Immunogenicity in Ferrets

The immunogenicity of rTET-NA was evaluated in influenza naïve and pre-immune ferrets. Ferrets are widely used in influenza research, based on their shared lung physiology and susceptibility to influenza with humans.

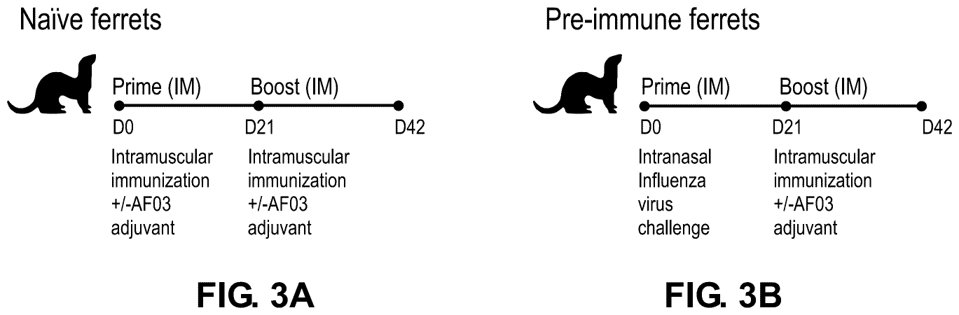

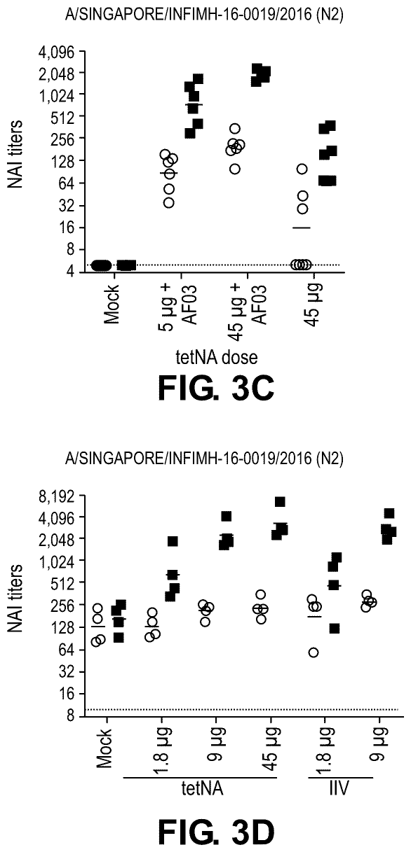

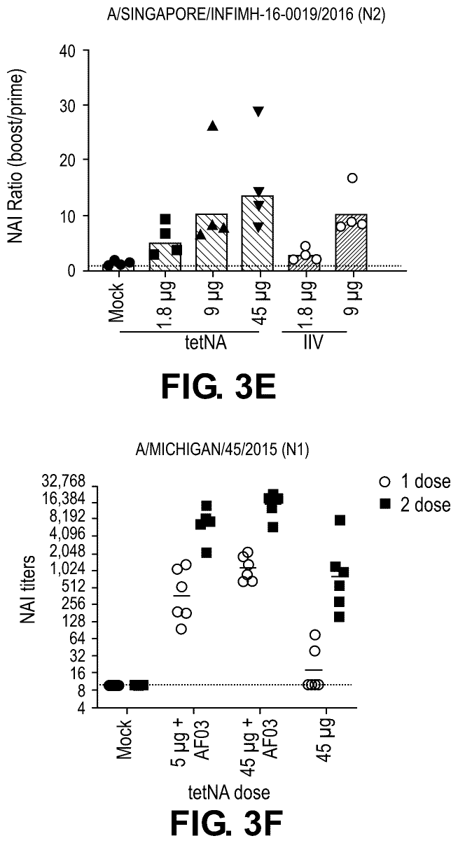

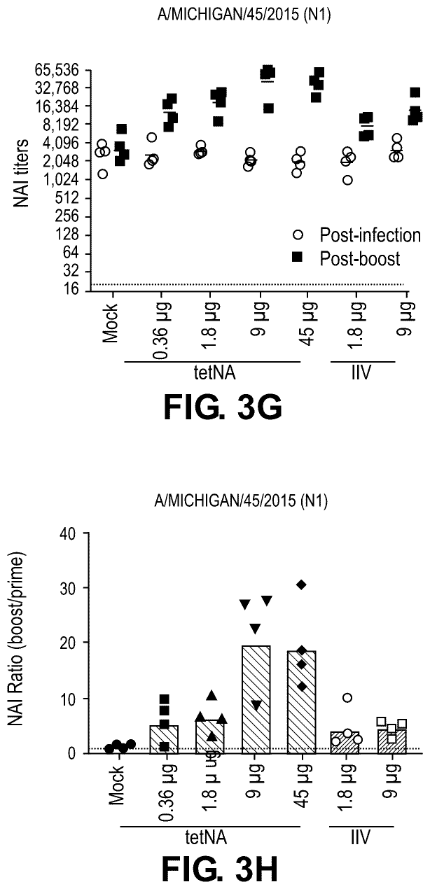

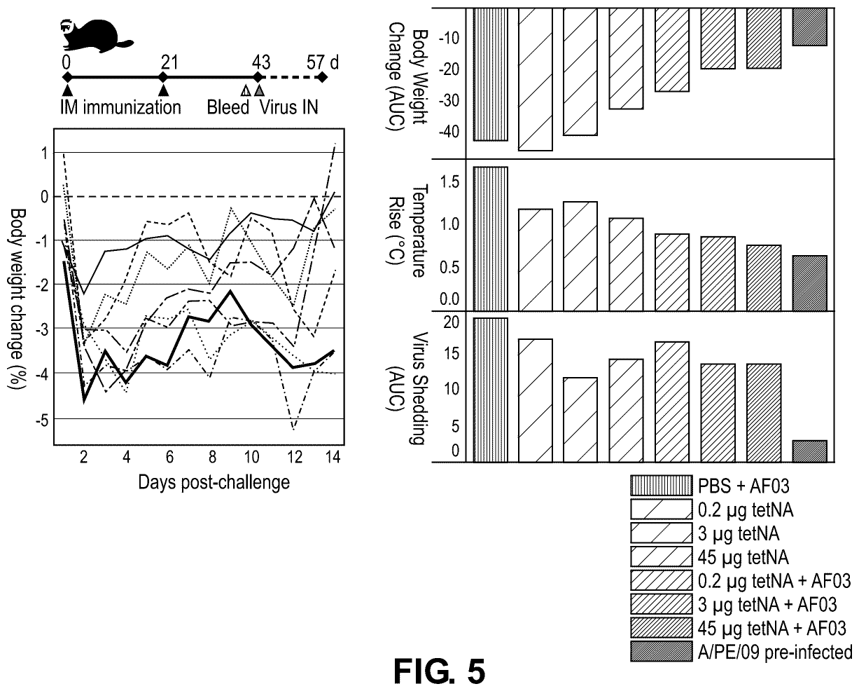

Naïve outbred male and female Fitch ferrets (6 per group) aged 17-21 weeks old were vaccinated intramuscularly twice 21 days apart (the study outline is illustrated in FIG. 3A) with either 5 μg+AF03, 45 μg+AF03, or 45 μg of N2 rTET-NA derived from A/Singapore/INFIMH-16-0019/2016 (FIG. 3C) or 5 μg+AF03, 45 μg+AF03, or 45 μg of N1 rTET-NA derived from A/Michigan/45/2015 (FIG. 3F) (500 μL/dose). As shown in FIG. 3B, in the pre-immune ferret model, ferrets were initially primed by intranasally-administered influenza virus (1,000 μL/dose, split evenly between nostrils) on Day 0. Three weeks after pre-immunization (Day 21), the ferrets were vaccinated with N2 rTET-NA derived from A/Singapore/INFIMH-16-0019/2016 (1.8 μg, 9 μg, or 45 μg), with IIV (1.8 μg or 9 μg), or with a vaccine diluent (mock) (FIG. 3D) or with N1 rTET-NA derived from A/Michigan/45/2015 (0.36 μg, 1.8 μg, 9 μg, or 45 μg), with IIV (1.8 μg or 9 μg), or with a vaccine diluent (mock) (FIG. 3G). NAI antibody titers were measured in sera three weeks after prime and boost.

All ferrets were bled under sedation at baseline, one day before or just before booster, and three weeks after booster. Sera samples (stored at −20° C. until required) were tested by ELLA to assess NAI activity.

It was demonstrated that rTET-NA was highly immunogenic in naïve ferrets and as a booster vaccine in pre-immune ferrets (FIGS. 3C-3H). rTET-NA was highly immunogenic in naïve ferrets after a single dose, which could be further boosted with a second dose (FIGS. 3C and 3F). As with the mouse model, NAI titers were enhanced with AF03 relative to the unadjuvanted formulation. The addition of adjuvant is dose sparing (e.g., 5 μg+AF03 is more immunogenic than 45 μg without adjuvant). Vaccination of pre-immune ferrets with a single dose of unadjuvanted A/Singapore/INFIMH-16-0019/2016 N2 rTET-NA or A/Michigan/45/2015 N1 rTET-NA resulted in comparable or superior boosting of NAI responses than matched IIV dose (FIGS. 3D and 3G).

Similar to naïve mice, rTET-N1 showed higher immunogenicity than IIV in the naïve ferret model at both doses tested, demonstrating expanded benefit of the rNA platform for N1 subtype (FIG. 3G). In the pre-immune ferret model, boosting of NAI responses following infection (NAI ratio boost/prime) was also greater with N1 at the two highest doses assessed (9 μg and 45 μg) than N2, but not at the lower doses assessed (1.8 μg). See FIGS. 3E and 3H.

### Example 4—Influenza Virus Challenge Studies in Ferrets

For influenza virus challenge studies, outbred male Fitch ferrets (16 per group) aged 17-21 weeks old naïve (Triple F farms, Sayre, PA) were initially vaccinated twice 21 days apart with the same dose (0.2 μg, 3 μg, or 45 μg) of A/Perth/09 N2 TET-NA (500 μL/dose, intramuscularly) with or without AF03 adjuvant. Three weeks after booster vaccination, the ferrets received, via intranasal administration, 107 PFU of A/Perth/09 H3N2 wild-type influenza A challenge virus (1,000 μL/dose, split evenly between nostrils). The animals were monitored for 14 days post-administration of the virus for clinical symptoms and changes in body weight once daily, and temperature twice daily.

Nasal washes were collected from all challenged animals on days 1, 3, 5 and 7 post-challenge and samples were stored at or below −65° C. for virus assessment. Ferrets were anesthetized with a ketamine (25 mg/kg) and xylazine (2 mg/kg) mixture, and 0.5 mL of sterile PBS containing penicillin (100 U/mL), streptomycin (100 μg/mL), and gentamicin (50 μg/mL) was injected into each nostril, collected and stored at or below −65° C. Virus in the nasal wash specimens was titrated by standard 50% tissue culture infectious dose (TCID₅₀) assay. The nasal washes were thawed and then clarified by centrifugation. The resulting supernatant was serially diluted 10-fold then transferred into respective wells of a 96-well plate which contains a monolayer of Madin-Darby Canine Kidney Cells (MDCK) cells for titration.

Sections of lungs (right and left cranial, and right and left caudal lung lobes) and nasal turbinates were harvested for viral titers on days 1, 3, 6, and 14 post-administration of virus. Tissue sections were weighed then flash frozen in an ethanol/dry ice bath or liquid nitrogen and stored at or below −65° C. for processing and virus titration by standard TCID₅₀ assay as discussed above. Selected ferrets (1-2) from each group were euthanized and necropsied on days 1, 3, 6 and 14 post-challenge. Lungs and nasal turbinates from necropsied ferrets were collected for viral titer and histopathology analyses. Lungs and nasal turbinates were fixed in 10% neutral buffered formalin. Scheduled necropsies for histopathology were supervised by a board-certified veterinary pathologist. Fixed lung lobes and NT sections were embedded in paraffin, processed by routine histologic methods, stained with hematoxylin and eosin, randomized, and graded for presence and severity of pathology by a board-certified veterinary pathologist.

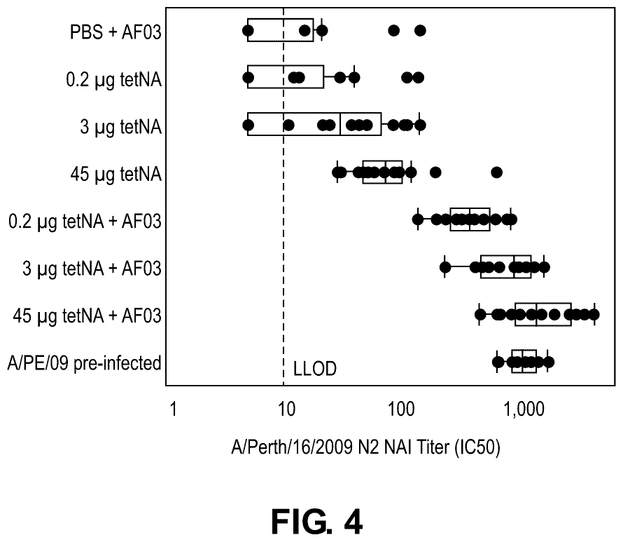

As shown for A/Singapore/2016 above, rTET-NA based on A/Perth/16/2009 N2 elicited dose-dependent NAI titers that were boosted by AF03. The highest rTET-NA dose assessed (45 μg) with AF03 induced superior titers than infection only at time of challenge (FIG. 4).

As shown in the FIG. 5, rTET-NA vaccination protected against disease severity following homologous H3N2 challenge in ferrets by reducing intensity and duration of clinical signs such as body weight loss and fever and overall viral shedding. NA-mediated protection was characterized by dose- and adjuvant-dependent reduction of overall body weight loss and peak temperature rise, comparable to pre-infection, only at high rTET-NA doses with AF03. Only a modest effect on total viral shedding was observed, which did not seem to follow a dose-dependent pattern (FIG. 5).

Individual ferret data analysis showed variability in the development of symptoms in mock AF03 group, not only in terms of intensity of symptoms but also in timing. While overall peak symptoms were observed on day 2 post-challenge, some animals developed late onset symptoms during the second week post-challenge or even a second peak of symptoms, making longitudinal analysis difficult.

Although rTET-NA does not appear to be as efficient as prior infection against viral shedding, this result is expected since infection provides both anti-NA and anti-HA immunity in addition to T-cell immunity against conserved epitopes.

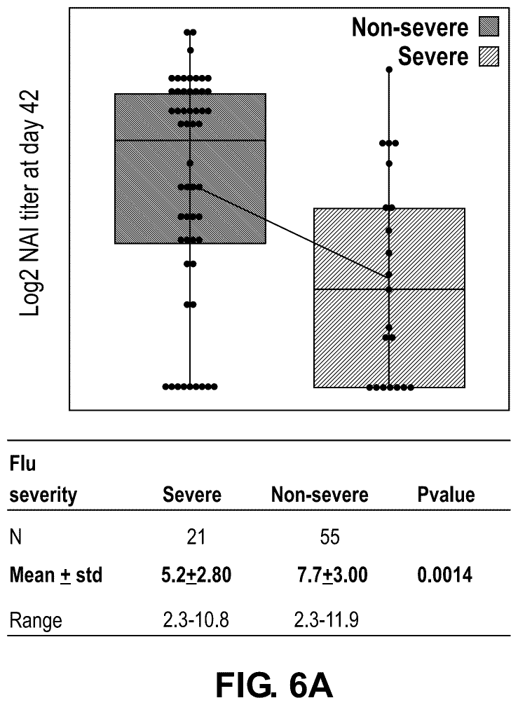

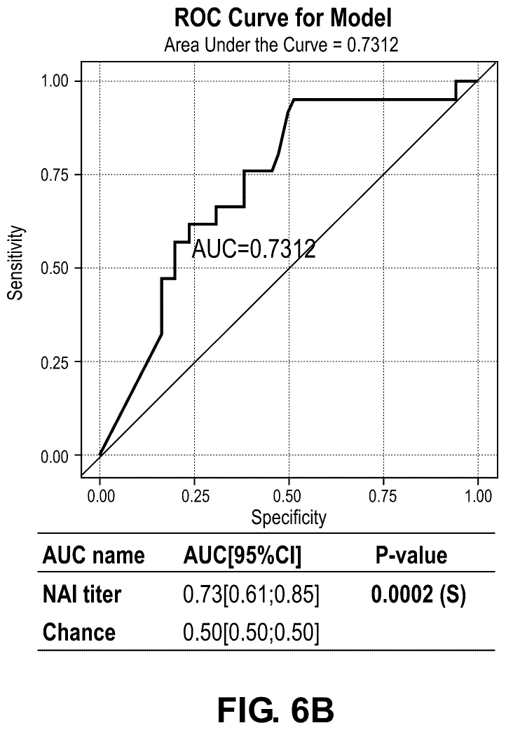

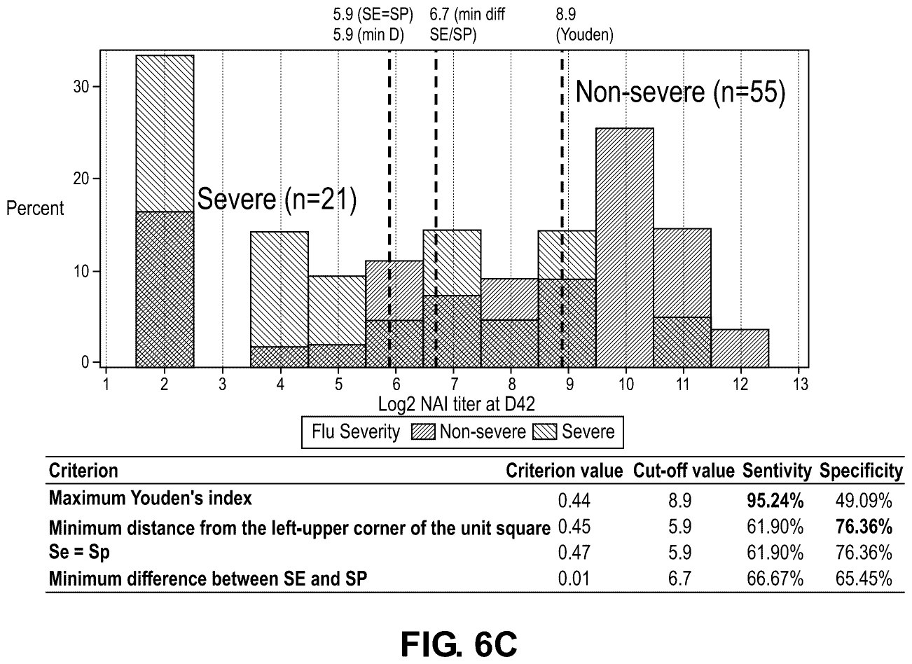

As shown in FIG. 6A-C, post-vaccination NAI titers were inversely correlated with disease severity and had the predictive power to be used as a correlate of protection. Animals exhibiting severe disease (n=21) had a mean Log2 NAI titer at Day 42 of 5.2±2 0.80, while animals exhibiting non-severe disease (n=55) had a mean Log3 NAI titer at Day 42 of 7.7±3.00 (p-value=0.0014) (FIG. 6A).

Association between anti-NA antibody responses and disease severity was examined by developing a disease scoring system based on peak symptoms and overall viral shedding. Ferrets were classified as severe and non-severe based on their combined severity score and distribution of NAI titers according to this binary disease classification was analyzed. Ferrets with severe symptoms showed significantly lower NAI titers than those classified as non-severe, though some non-responder ferrets were also protected (FIG. 6C). To further assess NAI as protective biomarker, receiver operating characteristics (ROC) analysis was developed. Area under the curve (AUC) of the ROC curve was significantly higher than chance (0.73 v. 0.50, p-value=0.0002), confirming that NAI titers could be used to correctly predict disease severity in ferrets (FIG. 6B). ROC analysis was also used to determine NAI titer threshold required for protection.

### Example 5—Evaluation of Multivalent HA and NA Immunogenicity in Mice

Mice were injected with a prime vaccine on Day 0 and a booster vaccine of the same dosage on Day 21. Blood was collected on Days 1, 20, 22, and 35. When AF03 adjuvant was used, it was mixed in a 1:1 ratio with antigens. A quadrivalent vaccine composition containing rTET-NA with each of N1, N2, NA from B/Victoria lineage, and NA from B/Yamagata lineage was used (specifically from strains A/Michigan/45/2015; A/Singapore/Infimh160019/2016; B/Colorado/06/2017; and B/Phuket/3037/2013), and a quadrivalent vaccine composition containing rHA with each of H1, H3, HA from B/Victoria lineage, and HA from B/Yamagata lineage was used (specifically from strains A/Michigan/45/2015; A/Singapore/Infimh160019/2016; B/Maryland/15/2016; and B/Phuket/3037/2013), as shown below in Table 1. For each group, n=6 mice. HAI titers were measured at Day 35 for the following influenza virus strains: A/Michigan/45/2015; A/Singapore/Infimh160019/2016; B/Maryland/15/2016; and B/Phuket/3037/2013. The results are reported below in Table 1:

TABLE 1
HA Immunogenicity for Recombinant Quadrivalent and Octavalent Vaccines

| μg per | μg per | | | | | |
|---|---|---|---|---|---|---|
| strain | strain | | Average HAI titer (n = 6) | | | |
| of tetNA | of rHA | Adjuv. | A/Michigan | A/Singapore | B/Maryland | B/Phuket |
| 0 | 0 | AF03 | <40 | <40 | <40 | <40 |
| — | 1 | — | 140 | <40 | <40 | <40 |
| 0.1 | — | AF03 | <40 | <40 | 40 | 66.7 |
| 1 | — | AF03 | <40 | 30 | 50 | 40 |
| — | 0.1 | AF03 | 2347 | 100 | 30 | 56.7 |
| — | 1 | AF03 | 1067 | 320 | 23.3 | 100 |
| 0.1 | 0.1 | AF03 | 1493 | 100 | 140 | 46.7 |
| 1 | 1 | AF03 | 1493 | 143 | 140 | 70 |

Likewise, NAI titers were similarly evaluated in mice with the following 4 strains of influenza virus: A/Michigan/45/2015; A/Singapore/Infimh160019/2016; B/Colorado/06/2017; and B/Phuket/3037/2013. The results are shown below in Table

TABLE 2
NA Immunogenicity for Recombinant Quadrivalent and Octavalent Vaccines

| μg per | μg per | | | | | |
|---|---|---|---|---|---|---|
| strain | strain | | Average Log2 IC50 NAI titer (n = 6) | | | |
| of tetNA | of rHA | Adjuv. | A/Michigan | A/Singapore | B/Colorado | B/Phuket |
| 0 | 0 | AF03 | 3.3 | 3.5 | 3.3 | 3.3 |
| — | 1 | — | 3.3 | 3.3 | 3.3 | 3.3 |
| 0.1 | — | AF03 | 6.4 | 4.0 | 6.4 | 5.1 |
| 1 | — | AF03 | 12.9 | 10.5 | 12.3 | 10.7 |
| — | 1 | AF03 | 3.3 | 3.3 | 3.3 | 3.3 |
| 0.1 | 0.1 | AF03 | 8.0 | 5.0 | 10.9 | 9.1 |
| 1 | 1 | AF03 | 11.7 | 8.3 | 11.2 | 10.3 |

As shown in Tables 1 and 2, vaccination with octavalent vaccine containing 4 rTET-NAs and 4 rHAs demonstrated HAI that was within 4-fold of the HAI for quadrivalent rHA for 3 out of 4 strains and greater than 4-fold for B/Maryland/15/2016. The octavalent vaccine showed improvement in the HAI as compared to vaccination with quadrivalent rHA alone, for example at a dosage of 1 μg/strain against B/Maryland/15/2016 (140 v. 23.3). Likewise, vaccination with octavalent vaccine containing 4 rTET-NAs and 4 rHAs demonstrated NAI that was within 4-fold of the NAI for quadrivalent rHA for 3 out of 4 strains; although NAI for A/Michigan/45/2015 dropped, the decrease was less than 4-fold.

### Example 6—Evaluation of Multivalent HA and NA Immunogenicity in Ferrets Part 1

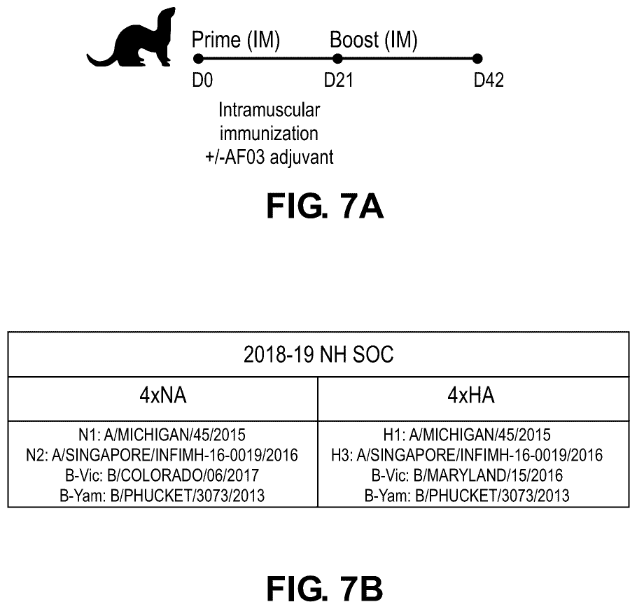

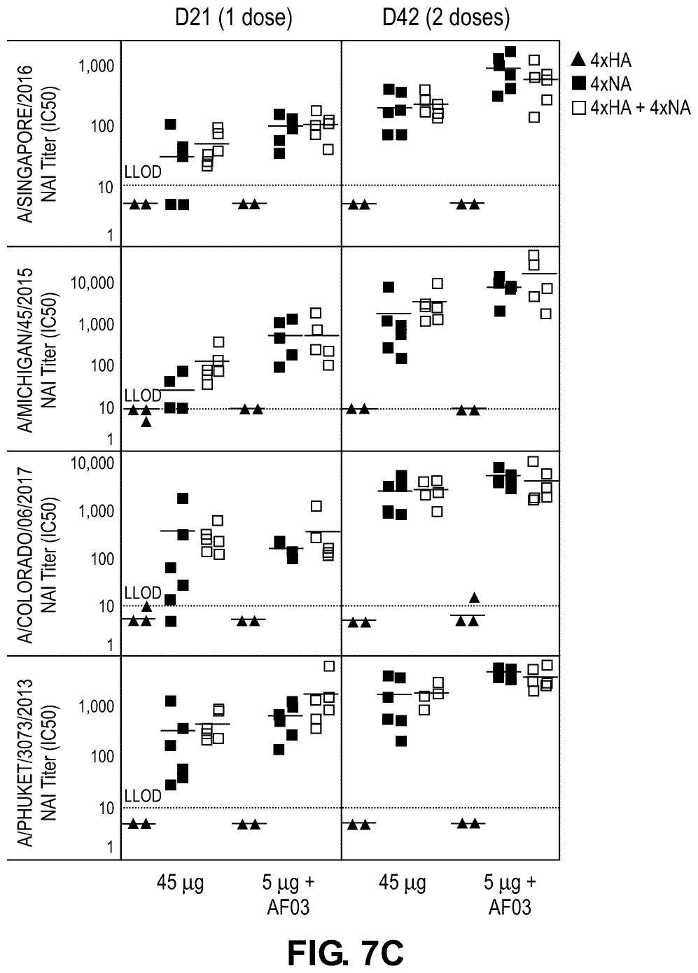

Naïve ferrets used to assess multivalent vaccine immunogenicity were vaccinated twice 21 days apart with an octavalent vaccine composition comprising a mixture of four rTET-NA antigens and/or four recombinant HA antigens, with and without adjuvant, as shown in FIG. 7A. This experiment also evaluated monovalent rTET-NA (i.e., rTET-NA derived from only A/Singapore/Infimh-16-0019/2016). The complete study design is shown in Table 3.

TABLE 3
Administration of Quadrivalent and Octavalent Vaccine Doses

| Group | NA source | HA source | Dose (μg) | Adjuvant |
|---|---|---|---|---|
| 1 (n = 6) | — | — | — | AF03 |
| 2 (n = 6) | rTET-NA | — | 5 μg each NA | AF03 |
| 3 (n = 6) | A/Singapore/Infimh- | | 45 μg each NA | AF03 |
| 4 (n = 6) | 16-0019/2016 (N2) | | 45 μg each NA | None |
| 5 (n = 6) | rTET-NA 2018-19 | — | 5 μg each NA | AF03 |
| 6 (n = 6) | SOC (N2, N1, Yam- | | 45 μg each NA | AF03 |
| 7 (n = 6) | B, Vic-B) | | 45 μg each NA | None |
| 8 (n = 6) | — | Baculo-rHA 2018- | 5 μg each NA | AF03 |
| 9 (n = 6) | | 19 SOC (H1, H3, | 45 μg each NA | None |
| | | Yam-B, Vic-B) | | |
| 10 (n = 6) | rTET-NA 2018-19 | Baculo-rHA 2018- | 5 μg each NA | AF03 |
| | SOC (N2, N1, Yam- | 19 SOC (H1, H3, | 5 μg each HA | |
| 11 (n = 6) | B, Vic-B) | Yam-B, Vic-B) | 45 μg each NA | None |
| | | | 45 μg each HA | |

Each rTET-NA antigen comprises the NA head domain from one of the standard of care strains included in the quadrivalent 2018-19 seasonal influenza vaccine (A/Singapore/INFIMH-16-0019/2016 (N2), A/Michigan/45/2015 (N1), B/Colorado/06/2017 (B/Victoria-lineage), and B/Phuket/3073/2013 (B/Yamagata-lineage)). Likewise, each recombinant HA includes HA from one of the following four strains: A/Michigan/45/2015 (H1); A/Singapore/Infimh-16-0019/2016 (H3); B/Maryland/15/2016 (B/Victoria lineage); and B/Phuket/3073/2013 (B/Yamagata lineage). FIG. 7B.

All ferrets were bled under sedation at baseline, one day before or just before booster, at booster vaccination, and three weeks after booster (day 42). Sera samples (stored at −20° C. until required) were tested by ELLA to assess NAI activity. Additionally, the hemagglutinin inhibition assay (HAI) and antibody forensics were also undertaken to assess antibody responses to hemagglutinin antigens following multivalent vaccination.

It was demonstrated that rTET-NA retains its immunogenicity following octavalent HA and NA vaccination in ferrets. This is the first demonstration of the potential of a fully recombinant octavalent vaccine containing HA and NA. Ferrets immunized with the recombinant octavalent composition developed NAI antibody response of comparable magnitude to animals immunized with a quadrivalent rNA composition alone, in a dose and adjuvant dependent manner (FIG. 7C). As shown in FIG. 7C, no interference was observed between HA and NA antigens, and some synergistic effects were detected for the 45 μg dose with no adjuvant after the first vaccination. Significant NAI titer increases were observed after the second vaccination. NAI titer (and NA ELISA) induction was confirmed across all four NA subtypes.

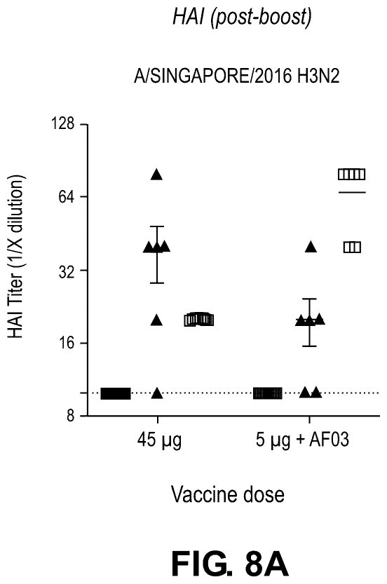

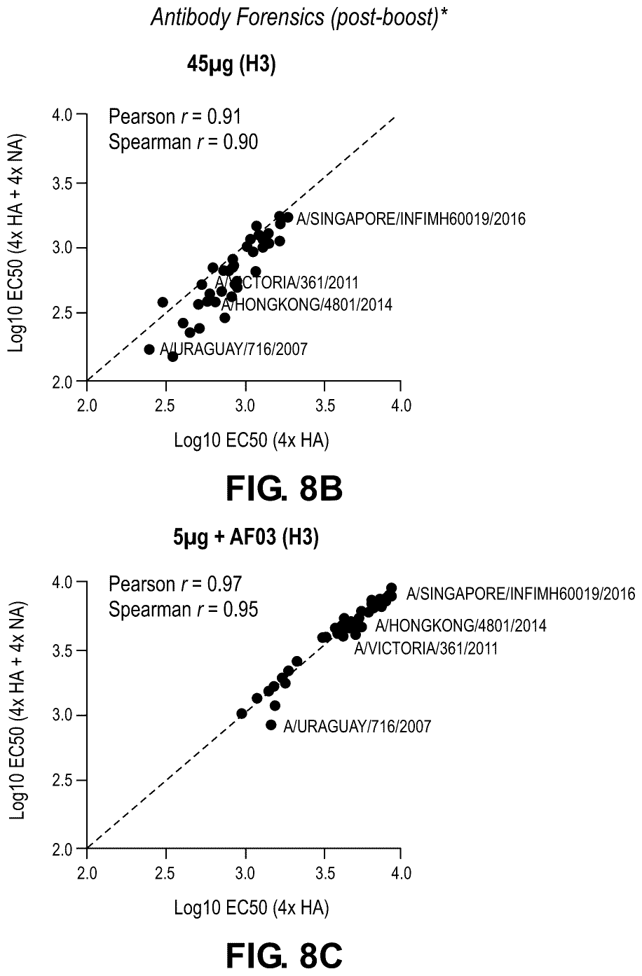

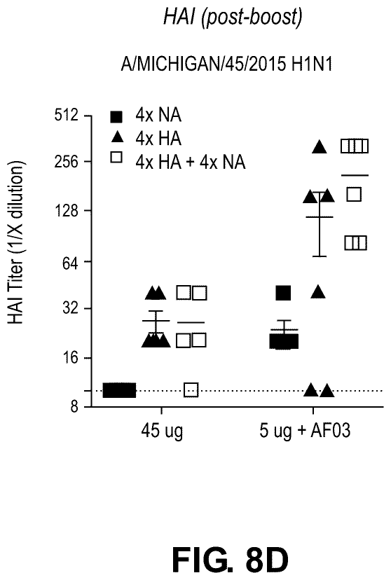

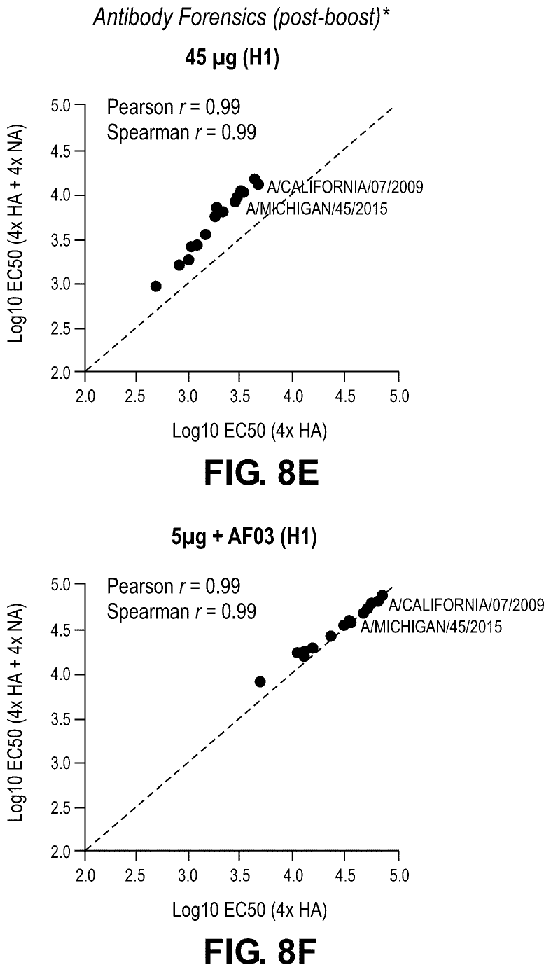

HA-specific antibody responses in octavalent vaccinated ferrets were comparable to ferrets immunized with a quadrivalent rHA vaccine alone, both in terms of quantity and quality as demonstrated by HAI and multiplex antibody-binding ELISA (antibody forensics, AF). As shown in FIG. 8, supplementation of quadrivalent rHA with quadrivalent rTET-NA does not interfere with the magnitude and breadth of the quadrivalent rHA immune response elicited for either H3 (FIG. 8A-C) or H1 (FIG. 8D-F). Results correspond to second immunization (post-boost) only. Similar results to NA above were demonstrated: there was a lack of interference between HA and NA antigens, and an apparent synergistic effect at the 5 μg+AF03 dose, as well as a dose-sparing effect of AF03. AF data demonstrating similar breadth of response elicited by HA antigens in quadrivalent rHA and octavalent formulations (4 rHA+4 rNA), as determined against panels of H1 and H3 HAs is shown in (FIG. 8B, C, E, F).

Thus, the addition of a quadrivalent recombinant influenza NA vaccine to a quadrivalent recombinant influenza HA vaccine induced robust NA-specific humoral immunity in ferrets without interfering with the ability to induce potent, HA-specific immunity.

The impact of an adjuvant (AF03) in this experiment on antibody responses induced by recombinant monovalent NA, quadrivalent rNA and octavalent rHA and rNA protein was also evaluated, and the results are shown below in Table 4.

TABLE 4
NAI response to mono- and multivalent vaccines

| | | Average Log2 IC50 NAI titer (n = 6) | | | | | | | |
|---|---|---|---|---|---|---|---|---|---|
| Vaccine | | A/Singapore | | A/Michigan | | B/Colorado | | B/Phuket | |
| (dose) | Adj. | Day 21 | Day 42 | Day 21 | Day 42 | Day 21 | Day 42 | Day 21 | Day 42 |
| Mock | AF03 | 2.32 | 2.32 | 3.32 | 3.32 | 2.32 | 2.70 | 2.32 | 2.32 |
| Monovalent | AF03 | 7.36 | 11.15 | 3.32 | 3.32 | 2.49 | 2.32 | 2.32 | 2.32 |
| NA (5 μg) | | | | | | | | | |
| Monovalent | AF03 | 8.44 | 11.66 | 3.32 | 3.32 | 2.32 | 2.32 | 2.32 | 2.32 |
| NA (45 μg) | | | | | | | | | |
| Monovalent | None | 4.33 | 7.45 | 3.32 | 3.32 | 2.32 | 2.32 | 2.32 | 2.32 |
| NA (45 μg) | | | | | | | | | |
| Quadrivalent | AF03 | 6.45 | 9.55 | 8.53 | 12.80 | 7.22 | 12.13 | 8.92 | 12.06 |
| NA (5 μg) | | | | | | | | | |
| Quadrivalent | AF03 | 7.64 | 10.91 | 10.10 | 13.91 | 9.59 | 13.81 | 10.15 | 12.74 |
| NA (45 μg) | | | | | | | | | |
| Quadrivalent | None | 3.99 | 7.34 | 4.16 | 9.62 | 5.99 | 10.78 | 7.02 | 9.97 |
| NA (45 μg) | | | | | | | | | |
| Quadrivalent | AF03 | 2.32 | 2.32 | 3.32 | 3.32 | 2.32 | 2.59 | 2.32 | 2.32 |
| HA (5 μg) | | | | | | | | | |
| Quadrivalent | None | 2.32 | 2.32 | 3.16 | 3.32 | 2.49 | 2.32 | 2.32 | 2.32 |
| HA (45 μg) | | | | | | | | | |
| Octavalent | AF03 | 6.54 | 8.89 | 8.25 | 13.15 | 7.91 | 11.56 | 10.05 | 11.56 |
| HA + NA (5 μg) | | | | | | | | | |
| Octavalent | None | 5.38 | 7.71 | 6.58 | 11.36 | 7.84 | 11.21 | 8.53 | 10.67 |
| HA + NA (45 μg) | | | | | | | | | |

AF03 adjuvant increases the NAI responses to mono- and multivalent NA vaccines and demonstrates a dose-sparing effect. A dose effect was observed after the first dose, and a large NAI boost (e.g., greater than 8-fold) was observed after the second dose for both the quadrivalent rNA and the octavalent rHA+rNA vaccines.

Furthermore, the addition of quadrivalent rHA to quadrivalent rNA does not reduce recombinant NA immunogenicity, independent of dose and/or adjuvant, and an apparent synergistic effect was observed after the first dose of octavalent rHA+rNA (45 μg) without an adjuvant as compared to quadrivalent rNA (45 μg) without adjuvant.

### Example 7—Evaluation of Multivalent HA and NA Immunogenicity in Ferrets Part 2

A quadrivalent vaccine composition containing rTET-NA with each of N1, N2, BvNA and ByNA (specifically from strains A/Michigan/45/2015; A/Singapore/Infimh160019/2016; B/Colorado/06/2017; and B/Phuket/3037/2013) was combined with a quadrivalent vaccine composition containing rHA with each of H1, H3, HBv, and HBy (specifically from strains A/Michigan/45/2015; A/Singapore/Infimh160019/2016; B/Maryland/15/2016; and B/Phuket/3037/2013), as shown below in Table 5. A quadrivalent vaccine composition containing rHA with each of H1, H3, HBv, and HBy and no adjuvant was used as a control. For each group, n=6 ferrets. mNT (HINT) titers were measured for the following influenza virus strains: A/Michigan/45/2015; A/Singapore/Infimh160019/2016; B/Iowa/06/2017; and B/Phuket/3037/2013. The results are reported below in Table 5. Ferrets were injected with a prime vaccine on Day 0 and a boost vaccine of the same dosage on Day 21. Blood was collected on Days −7, 1, 20, 22, and 42. When adjuvant was used, it was mixed in a 1:1 ratio with antigens.

TABLE 5
HA Immunogenicity for Recombinant Tetravalent and Octavalent Vaccines

| μg per | μg per | | | | | |
|---|---|---|---|---|---|---|
| strain | strain | | Average HINT mNT titer (n = 6) | | | |
| of tetNA | of rHA | Adjuv. | A/Michigan | A/Singapore | B/Iowa | B/Phuket |
| 0 | 0 | — | <20 | <20 | <20 | <20 |
| 0 | 0 | AF03 | <20 | <20 | <20 | <20 |
| 0 | 0 | SPA14HD | <20 | <20 | <20 | <20 |
| 35 | 35 | — | 2207 | 276 | 26.3 | 69.1 |
| 1 | 1 | AF03 | 2813 | 470 | 116.7 | 203.5 |
| 5 | 5 | AF03 | 2915 | 468 | 71.3 | 333.9 |
| 1 | 1 | SPA14LD | 3000 | 197 | 64.3 | 192.8 |
| 5 | 5 | SPA14LD | 2516 | 321 | 58 | 177.9 |
| 1 | 1 | SPA14HD | 3000 | 1430 | 101.3 | 358 |
| 5 | 5 | SPA14HD | 3000 | 776 | 127.6 | 327.3 |
| — | 35 | — | 1985 | 90 | 11.7 | 77.9 |

As shown above, all three adjuvants (AF03, SPA14LD (low dose), and SPA14HD (high dose)) showed comparable or improved titers over compositions containing no adjuvant. No significant difference was observed between adjuvant responses, even at low doses. The octavalent recombinant vaccine administration with adjuvant elicited comparable (within 4-fold) or increased (greater than 4-fold) responses over the quadrivalent recombinant HA vaccine administration at the highest dose of 35 μg without adjuvant.

Likewise, NAI titers were similarly evaluated in ferrets with the following 4 strains of influenza virus: A/Michigan/45/2015; A/Singapore/Infimh16009/2016; B/Colorado/06/2017; and B/Phuket/3037/2013. The results are shown below in Table 6.

TABLE 6
NAI Response in Ferrets at Day 42

| μg per | μg per | | | | | |
|---|---|---|---|---|---|---|
| strain | strain | | Average Log2 IC50 NAI titer (n = 6) | | | |
| of tetNA | of rHA | Adjuv. | A/Michigan | A/Singapore | B/Colorado | B/Phuket |
| 0 | 0 | — | 2.3 | 2.3 | 2.3 | 2.3 |
| 0 | 0 | AF03 | 2.3 | 2.3 | 2.3 | 2.3 |
| 0 | 0 | SPA14HD | 2.3 | 2.3 | 2.3 | 2.3 |
| 35 | 35 | — | 11.6 | 6.1 | 9.7 | 9.1 |
| 1 | 1 | AF03 | 11.6 | 7.8 | 10.9 | 10.4 |
| 5 | 5 | AF03 | 11.7 | 7.9 | 10.9 | 10.3 |
| 1 | 1 | SPA14LD | 12.7 | 8.5 | 12.2 | 12.0 |
| 5 | 5 | SPA14LD | 12.8 | 8.3 | 11.4 | 10.9 |
| 1 | 1 | SPA14HD | 13.3 | 8.2 | 12.7 | 12.3 |
| 5 | 5 | SPA14HD | 13.0 | 9.1 | 12.6 | 11.9 |
| — | 35 | — | 2.3 | 2.3 | 2.3 | 2.3 |

As shown above, it was observed that all three adjuvants (AF03, SPA1, and SPA14LD) improved NAITresponses significantly over no adjuvant against 3 of the 4 strains tested (A/Singapore/Infimh160019/2016; B/Colorado/06/2017; and B/Phuket/3037/2013).

It was further observed that SPA14HD induced the most significant increases in NAI response over no adjuvant and improved B-strain NAI significantly even at lower dosages after one immunization, as shown in Table 7 below, which provides the NAI response at Day 20, before a booster dose was administered.

TABLE 7
NAI Response in Ferrets at Day 20

| μg per | μg per | | | | | |
|---|---|---|---|---|---|---|
| strain | strain | | Average Log2 IC50 NAI titer (n = 6) | | | |
| of tetNA | of rHA | Adjuv. | A/Michigan | A/Singapore | B/Colorado | B/Phuket |
| 0 | 0 | — | 3.9 | 3.1 | 2.3 | 2.3 |
| 0 | 0 | AF03 | 2.8 | 3.1 | 2.3 | 2.3 |
| 0 | 0 | SPA14HD | 3.6 | 4.1 | 2.3 | 2.3 |
| 35 | 35 | — | 6.5 | 5.4 | 5.9 | 7.5 |
| 1 | 1 | AF03 | 3.5 | 6.1 | 7.4 | 8.5 |
| 5 | 5 | AF03 | 6.4 | 7.3 | 8.1 | 9.8 |
| 1 | 1 | SPA14LD | 5.9 | 6.4 | 8.8 | 11.4 |
| 5 | 5 | SPA14LD | 7.4 | 6.9 | 8.8 | 10.1 |
| 1 | 1 | SPA14HD | 7.9 | 6.8 | 9.8 | 11.6 |
| 5 | 5 | SPA14HD | 8.5 | 7.9 | 9.4 | 10.9 |
| — | 35 | — | 3.0 | 4.3 | 2.4 | 2.3 |

### Example 8—Induction of Broad NAT Immunogenicity with Multivalent HA and NA Vaccine Images in Ferrets

A quadrivalent vaccine composition containing rTET-NA with each of N1, N2, BvNA and ByNA (specifically from strains A/Michigan/45/2015; A/Singapore/Infimh160019/2016; B/Colorado/06/2017; and B/Phuket/3037/2013) was combined with a quadrivalent vaccine composition containing rHA with each of H1, H3, HBv, and HBy (specifically from strains A/Michigan/45/2015; A/Singapore/InfimTh160019/2016; B/Maryland/15/2016; and B/Phuket/3037/2013), as shown below in Table 8. NAI titers were measured for the following influenza virus strains: A/Singapore/Infimh160019/2016; A/Hatay/4990/2016; A/Sweden/3/2017; A/Louisiana/13/2017; A/Townsville51/2016; A/Aksaray/4048/2016; A/Perth/16/2009; and A/Ohio13/2017. The results are reported below in Table 8. Ferrets were injected with a prime vaccine on Day 0 and a boost vaccine of the same dosage on Day 21. Blood was collected on Days 1, 20, 22, and 42.

TABLE 8
NAI Response in Ferrets

| μg per | μg per | | | | | |
|---|---|---|---|---|---|---|
| strain | strain | | Average Log2 IC50 NAI titer (n = 6) | | | |
| of tetNA | of rHA | Adjuv. | A/Singapore | A/Hatay | A/Sweden | A/Louisiana |
| 0 | 0 | SPA14HD | 5 | 6 | 5 | 18 |
| 0 | 0 | — | 5 | 5 | 6 | 6 |
| 0 | 0 | AF03 | 5 | 5 | 6 | 21 |
| 5 | 5 | SPA14HD | 562 | 1191 | 466 | 829 |
| 5 | 5 | SPA14LD | 305 | 898 | 403 | 426 |
| 5 | 5 | AF03 | 236 | 479 | 318 | 353 |
| 35 | 35 | — | 69 | 138 | 91 | 139 |

Positive control

| — | — | — | 1714 | 3257 | 1805 | 1048 |
|---|---|---|---|---|---|---|
| | | | A/Townsville | A/Aksaray | A/Perth | A/Ohio |
| 0 | 0 | SPA14HD | 22 | 55 | 5 | 5 |
| 0 | 0 | — | 20 | 7 | 5 | 5 |
| 0 | 0 | AF03 | 43 | 18 | 5 | 5 |
| 5 | 5 | SPA14HD | 450 | 758 | 266 | 7 |
| 5 | 5 | SPA14LD | 324 | 442 | 241 | 6 |
| 5 | 5 | AF03 | 149 | 143 | 170 | 8 |
| 35 | 35 | — | 114 | 42 | 226 | 5 |

Positive control

| | | | | | | |
|---|---|---|---|---|---|---|
| — | — | — | 2729 | 506 | 2732 | 5852 |

As shown above, broad NAI responses were demonstrated across strains after administration of the octavalent recombinant vaccine combination. A trend of higher titers was observed with the SPA14HD adjuvant against all heterologous N2 strains tested.

### Example 9—Multivalent HA and NA Vaccine Images in a Pre-Immune Ferret Model

After confirmation of flu negative HAI status, pre-immune ferrets were pre-immunized intranasally on Day 0 with a mixture of the following four live virus imprinting strains [1×10⁵ ffu/strain; 0.5 mL per nostril (1 mL total)]: A/NewCaledonia/20/1999; A/Perth/16/2009; B/HongKong330/2001; and B/Florida/4/2006. On Day 21, ferrets were immunized with an octavalent recombinant protein vaccine composition containing rTET-NA with each of N1, N2, BvNA and ByNA (specifically from strains A/Michigan/45/2015; A/Singapore/Infimh160019/2016; B/Colorado/06/2017; and B/Phuket/3037/2013) and rHA with each of H1, H3, HBv, and HBy (specifically from strains A/Michigan/45/2015; A/Singapore/Infimh160019/2016; B/Maryland/15/2016; and B/Phuket/3037/2013), as shown below in Table 9. Blood was drawn on Day 20 to establish baseline titers, and blood was drawn again on Day 42 to measure ELLA antibody responses (Day 21 post recombinant protein immunization). The results are shown in Table 9 below, and the Average IC50 ratio for each group is shown below in Table 10.

TABLE 9
ELLA Antibody Responses in Ferrets

| μg per | μg per | | Average Log2 IC50 ELLA titer (n = 6) | | | | |
|---|---|---|---|---|---|---|---|
| strain | strain | | A/Mich | A/Mich | B/Colo | B/Colo | B/Phu |
| of tetNA | of rHA | Adjuv. | Day 20 | Day 42 | Day 20 | Day 42 | Day 20 |
| 0 | 0 | — | 2.3 | 2.7 | 2.3 | 2.7 | 7.2 |
| 0 | 0 | AF03 | 2.3 | 2.3 | 2.3 | 2.3 | 7.7 |
| 0 | 0 | SPA14HD | 3.5 | 3.6 | 3.5 | 3.6 | 7.8 |
| 35 | 35 | — | 2.3 | 8.8 | 2.3 | 8.8 | 7.1 |
| 5 | 5 | AF03 | 2.3 | 8.6 | 2.3 | 8.6 | 7.3 |
| 5 | 5 | SPA14LD | 2.3 | 8.0 | 2.3 | 8.0 | 7.4 |
| 5 | 0 | SPA14HD | 2.3 | 8.9 | 2.3 | 8.9 | 7.7 |
| 0 | 5 | SPA14HD | 2.2 | 3.0 | 2.2 | 3.0 | 7.5 |
| 5 | 5 | SPA14HD | 2.4 | 8.8 | 2.4 | 8.8 | 7.4 |
| — | 15 (QIV | — | 2.7 | 5.0 | 2.7 | 5.0 | 8.1 |
| | Control) | | | | | | |
| μg per | μg per | | | | | | |
| strain | strain | | B/Phu | A/Sing | A/Sing | A/Perth | A/Perth |
| of tetNA | of rHA | Adjuv. | Day 42 | Day 20 | Day 42 | Day 20 | Day 42 |
| 0 | 0 | — | 8.2 | 4.4 | 5.9 | 8.7 | 9.0 |
| 0 | 0 | AF03 | 7.8 | 4.5 | 6.3 | 8.5 | 9.1 |
| 0 | 0 | SPA14HD | 7.2 | 3.4 | 6.4 | 8.7 | 8.9 |
| 35 | 35 | — | 8.9 | 5.6 | 8.8 | 8.1 | 10.0 |
| 5 | 5 | AF03 | 8.8 | 4.9 | 9.1 | 8.5 | 10.2 |
| 5 | 5 | SPA14LD | 8.7 | 5.3 | 9.0 | 8.4 | 10.5 |
| 5 | 0 | SPA14HD | 9.8 | 4.9 | 9.8 | 8.3 | 10.6 |
| 0 | 5 | SPA14HD | 6.6 | 4.8 | 6.5 | 9.0 | 9.1 |
| 5 | 5 | SPA14HD | 9.2 | 4.3 | 9.4 | 8.5 | 10.5 |
| — | 15 (QIV | — | 8.0 | 5.0 | 8.1 | 8.8 | 9.5 |
| | Control) | | | | | | |

TABLE 10
Average IC50 Ratio of ELLA Antibody Responses in Ferrets

| μg per | μg per | | | | | | |
|---|---|---|---|---|---|---|---|
| strain | strain | | Average IC50 Ratio of ELLA Titers (n = 6) | | | | |
| of tetNA | of rHA | Adjuv. | A/Mich | B/Colo | B/Phu | A/Sing | A/Perth |
| 0 | 0 | — | 1.73 | 1.73 | 2.0 | 4.0 | 1.3 |
| 0 | 0 | AF03 | 1.00 | 1.00 | 1.2 | 5.5 | 1.7 |
| 0 | 0 | SPA14HD | 2.46 | 2.46 | 0.7 | 12.5 | 1.2 |
| 35 | 35 | — | 92.68 | 92.68 | 4.2 | 10.6 | 4.0 |
| 5 | 5 | AF03 | 92.73 | 92.73 | 3.2 | 25.2 | 3.6 |
| 5 | 5 | SPA14LD | 62.50 | 62.50 | 2.6 | 15.4 | 4.7 |
| 5 | 0 | SPA14HD | 96.20 | 96.20 | 4.4 | 57.6 | 6.1 |
| 0 | 5 | SPA14HD | 2.53 | 2.53 | 0.6 | 5.7 | 1.0 |
| 5 | 5 | SPA14HD | 93.14 | 93.14 | 5.1 | 46.9 | 4.4 |
| — | 15 (QIV | — | 7.33 | 7.33 | 1.1 | 12.0 | 1.7 |
| | Control) | | | | | | |

As shown in the Tables 9 and 10 above, the recombinant octavalent vaccine composition elicited a strong ELLA response against A/Michigan/45/2015, B/Colorado/06/2017, and A/Singapore/Infimh160019/2016 regardless of adjuvant used. A weaker (inconclusive) response was observed against A/Perth/16/2009, and the response against B/Phuket/3037/2013 was more difficult to detect because of an initially high baseline. Therefore, it was observed that infection in pre-immune ferrets induced some level of cross-reactive ELLA responses against most 2018/2019 SOC strains that were boosted by a single administration of the octavalent 2018/2019 SOC strain protein vaccine.

Additionally, HA neutralization titers were measured using the HINT mNT protocol. The results are shown below in Table 11.

TABLE 11
HA Neutralization Titers in Ferrets

| μg | | | | | |
|---|---|---|---|---|---|
| per | | | Log2 EC50 HINT Titers (n = 6) | | |
| strain | μg per | | | | A/Singapore |
| of | strain | | A/Singapore | A/Singapore | Average EC50 |
| tetNA | of rHA | Adjuv. | Day 20 | Day 42 | Ratio |
| 0 | 0 | — | 8.1 | 10.2 | 4.8 |
| 0 | 0 | AF03 | 7.5 | 9.8 | 5.4 |
| 0 | 0 | SPA14HD | 7.6 | 9.2 | 4.0 |
| 35 | 35 | — | 7.1 | 10.2 | 11.7 |
| 5 | 5 | AF03 | 7.0 | 11.2 | 21.7 |
| 5 | 5 | SPA14LD | 7.2 | 10.3 | 9.5 |
| 5 | 0 | SPA14HD | 7.0 | 8.5 | 3.0 |
| 0 | 5 | SPA14HD | 6.6 | 10.7 | 19.4 |
| 5 | 5 | SPA14HD | 7.2 | 11.4 | 34.4 |
| — | 15 (QIV | — | 8.0 | 11.1 | 8.9 |
| | Control) | | | | |
| | | | A/Perth | A/Perth | A/Perth Average |
| | | | Day 20 | Day 42 | EC50 Ratio |
| 0 | 0 | — | 11.7 | 11.2 | 0.8 |
| 0 | 0 | AF03 | 11.3 | 10.8 | 0.8 |
| 0 | 0 | SPA14HD | 11.0 | 10.6 | 0.9 |
| 35 | 35 | — | 11.5 | 10.6 | 0.6 |
| 5 | 5 | AF03 | 10.7 | 11.3 | 1.5 |
| 5 | 5 | SPA14LD | 11.1 | 11.2 | 1.2 |
| 5 | 0 | SPA14HD | 11.3 | 10.8 | 0.8 |
| 0 | 5 | SPA14HD | 11.0 | 11.3 | 1.3 |
| 5 | 5 | SPA14HD | 11.5 | 11.9 | 1.7 |
| — | 15 (QIV | — | 11.7 | 11.8 | 1.4 |
| | Control) | | | | |
| | | | A/Phuket | A/Phuket | A/Phuket Average |
| | | | Day 20 | Day 42 | EC50 Ratio |
| 0 | 0 | — | 8.9 | 10.1 | 2.5 |
| 0 | 0 | AF03 | 8.4 | 9.8 | 3.1 |
| 0 | 0 | SPA14HD | 8.6 | 9.8 | 2.4 |
| 35 | 35 | — | 8.2 | 9.8 | 3.3 |
| 5 | 5 | AF03 | 8.7 | 9.9 | 3.7 |
| 5 | 5 | SPA14LD | 8.4 | 9.8 | 2.6 |
| 5 | 0 | SPA14HD | 8.4 | 9.7 | 2.8 |
| 0 | 5 | SPA14HD | 8.4 | 10.3 | 3.7 |
| 5 | 5 | SPA14HD | 8.4 | 10.1 | 3.8 |
| — | 15 (QIV | — | 8.4 | 10.4 | 4.4 |
| | Control) | | | | |

As shown in Table 11, similar HINT titers were observed across all groups post-immunization with the octavalent vaccine composition.

### Example 10—Protection of Multivalent Recombinant Vaccine in Mice Against Homologous and Drifted H1N1

Mice were immunized on Day 0 (prime) and Day 21 (booster) with a recombinant octavalent vaccine composition containing rTET-NA with each of N1, N2, BvNA and ByNA (specifically from strains A/Michigan/45/2015; A/Singapore/Infimh160019/2016; B/Colorado/06/2017; and B/Phuket/3037/2013) and rHA with each of H1, H3, HBv, and HBy (specifically from strains A/Michigan/45/2015; A/Singapore/Infimh160019/2016; B/Maryland/15/2016; and B/Phuket/3037/2013), in a dosage amount of 0.2 μg/strain. Control mice were administered PBS. N=5 for both groups. Blood was drawn three weeks after administration of the prime and three weeks after administration of the booster. Mice were challenged on Day 42 with either 5LD₅₀ (5 times the lethal dose inducing 50% mortality) of A/Belgium/145/2009 or 5LD₅₀ of Wisconsin/588/2019, and change in weight was monitored.

It was demonstrated that mice were well protected from body weight loss during the two week monitoring period post infection with 5LD₅₀ of A/Belgium/145/2009 as compared to control mice. Vaccinated mice had a 100% survival rate, as compared to the control mice having a 100% mortality by the eighth day after infection.

Furthermore, it was demonstrated that mice were better protected from body weight loss for up to two weeks post infection with 5LD₅₀ of Wisconsin/588/2019 as compared to control mice, although both vaccinated and control mice had a 100% survival rate.

It is also noted that, as used in this disclosure and the appended claims, the singular forms “a”, “an”, and “the” include plural referents unless the context clearly dictates otherwise. Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination). Ranges may be expressed herein as from about one particular value, and/or to about another particular value. When such a range is expressed, another aspect includes from the one particular value and/or to the other particular value. Similarly, when values are expressed as approximations, by use of the antecedent about, it will be understood that the particular value forms another aspect. It will be further understood that the endpoints of each of the ranges are significant both in relation to the other endpoint, and independently of the other endpoint. All references cited in this disclosure are hereby incorporated herein in their entirety.

What is claimed is:

1. An immunogenic composition comprising a plurality of recombinant influenza virus proteins, wherein the plurality of recombinant influenza virus proteins comprises:
a first recombinant influenza virus hemagglutinin (HA), wherein the first recombinant influenza virus HA is an H1 HA;
a second recombinant influenza virus HA, wherein the second recombinant influenza virus HA is an H3 HA;
a third recombinant influenza virus HA, wherein the third recombinant influenza virus HA is from a B/Victoria lineage;
a fourth recombinant influenza virus HA, wherein the fourth recombinant influenza virus HA is from a B/Yamagata lineage;
a first recombinant influenza virus neuraminidase (NA), wherein the first recombinant influenza virus NA is an N1 NA;
a second recombinant influenza virus NA, wherein the second recombinant influenza virus NA is an N2 NA;
a third recombinant influenza virus NA, wherein the third recombinant influenza virus NA is from a B/Victoria lineage; and
a fourth recombinant influenza virus NA, wherein the fourth recombinant influenza virus NA is from a B/Yamagata lineage.

2. The immunogenic composition according to claim 1, wherein each of the first, second, third, and fourth recombinant influenza virus NA is a modified recombinant influenza virus NA.

3. The immunogenic composition according to claim 2, wherein the modified recombinant influenza virus NA comprises a modified recombinant tetrameric influenza virus NA comprising four modified monomeric NA molecules, each comprising a head region of the NA of the influenza virus, but lacking a cytoplasmic tail, a transmembrane region, and all or substantially all of a stalk region of the NA of the influenza virus and wherein the modified monomeric NA molecules form modified recombinant tetrameric NA when expressed in a host cell.

4. The immunogenic composition according to claim 3, wherein each modified recombinant monomeric influenza virus NA comprises a heterologous tetramerization domain.

5. The immunogenic composition according to claim 3, wherein each modified recombinant monomeric influenza virus NA does not comprise a heterologous oligomerization domain.

6. The immunogenic composition according to claim 4, wherein the heterologous tetramerization domain is a Staphylothermus marinus tetrabrachion tetramerization domain, a GCN4 leucine zipper tetramerization domain, a tetramerization domain from a paramyxovirus phosphoprotein, or a human vasodilator stimulated phosphoprotein (VASP) tetramerization domain.

7. The immunogenic composition according to any one of claims 1-6, wherein each of the recombinant influenza virus HA is produced by a baculovirus expression system in cultured insect cells.

8. The immunogenic composition according to any one of claims 1-7, wherein each of the recombinant influenza virus NA is produced in Chinese Hamster Ovary (CHO) cells.

9. The immunogenic composition according to any one of claims 1-8, wherein the immunogenic composition does not contain inactivated influenza virions or live attenuated influenza virions.

10. The immunogenic composition according to any one of claims 1-9, wherein each of the recombinant influenza virus HAs and/or each of the recombinant influenza virus NAs are from standard of care influenza strains.

11. The immunogenic composition according to any one of claims 1-10, wherein the H1 HA is from an H1N1 influenza virus strain and/or the H3 HA is from an H3N2 influenza virus strain.

12. The immunogenic composition according to any one of claims 1-11, wherein the N1 NA is from an H1N1 influenza virus strain and/or the N2 NA is from an H3N2 influenza virus strain.

13. The immunogenic composition according to any one of claims 1-12, wherein the H1 HA is from an H1N1 influenza virus strain, the H3 HA is from an H3N2 influenza virus strain, the N1 NA is from an H1N1 influenza virus strain, and the N2 NA is from an H3N2 influenza virus strain.

14. The immunogenic composition of claim 13, wherein the H1 HA and the N1 NA are from the same H1N1 influenza virus strain and the H3 HA and N2 NA are from the same H3N2 influenza virus strain.

15. The immunogenic composition according to any one of claims 1-14, wherein the plurality of recombinant influenza virus proteins consists of:
a first recombinant influenza virus HA, wherein the first recombinant influenza virus HA is an H1 HA;
a second recombinant influenza virus HA, wherein the second recombinant influenza virus HA is an H3 HA;
a third recombinant influenza virus HA, wherein the third recombinant influenza virus HA is from a B/Victoria lineage;
a fourth recombinant influenza virus HA, wherein the fourth recombinant influenza virus HA is from a B/Yamagata lineage;
a first recombinant influenza virus NA, wherein the first recombinant influenza virus NA is an N1 NA;
a second recombinant influenza virus NA, wherein the second recombinant influenza virus NA is an N2 NA;
a third recombinant influenza virus NA, wherein the third recombinant influenza virus NA is from a B/Victoria lineage; and
a fourth recombinant influenza virus NA, wherein the fourth recombinant influenza virus NA is from a B/Yamagata lineage.

16. The immunogenic composition according to any one of claims 1-15, wherein the composition further comprises an adjuvant.

17. The immunogenic composition according to claim 16, wherein the adjuvant comprises a squalene-in-water adjuvant or a liposome-based adjuvant.

18. The immunogenic composition according to claim 17, wherein the squalene-in-water adjuvant comprises AF03.

19. The immunogenic composition according to claim 17, wherein the liposome-based adjuvant comprises SPA14.

20. The immunogenic composition according to any one of claims 1-19, wherein each of the recombinant influenza virus HAs is present in the composition in an amount ranging from about 0.1 μg to about 90 μg, optionally about 1 μg to about 60 μg or 5 μg to about 45 μg.

21. The immunogenic composition according to any one of claims 1-20, wherein each of the recombinant influenza virus NAs is present in the composition in an amount ranging from about 0.1 μg to about 90 μg, optionally about 1 μg to about 60 μg or about 5 μg to about 45 μg.

22. The immunogenic composition according to any one of claims 1-21, wherein the composition is formulated for intramuscular injection.

23. A vaccine comprising the immunogenic composition according to any one of claims 1-22 and a pharmaceutical carrier.

24. A method of immunizing a subject against influenza virus, the method comprising administering to the subject an immunologically effective amount of the vaccine of claim 23.

25. The method of claim 24, wherein the method prevents influenza virus infection in the subject.

26. The method of claim 24 or 25, wherein the method raises a protective immune response in the subject.

27. The method of claim 26, wherein the protective immune response comprises an HA antibody response and/or an NA antibody response.

28. The method of any one of claims 24-27, wherein the subject is human.

29. The method of any one of claims 24-28, wherein the vaccine is administered intramuscularly, intradermally, subcutaneously, intravenously, intranasally, by inhalation, or intraperitoneally.

30. The method of any one of claims 24-29, wherein the method treats or prevents disease caused by either or both a seasonal and a pandemic influenza strain.

31. The method of any one of claims 24-30, wherein the subject is human and the human is 6 months of age or older, less than 18 years of age, at least 6 months of age and less than 18 years of age, at least 18 years of age and less than 65 years of age, at least 6 months of age and less than 5 years of age, at least 5 years of age and less than 65 years of age, at least 60 years of age, or at least 65 years of age.

32. A method of reducing one or more symptoms of influenza virus infection, the method comprising administering to a subject a prophylactically effective amount of the vaccine of claim 23.

33. A method of enhancing or broadening a protective immune response in a subject, the method comprising administering to the subject an immunologically effective amount of the vaccine according to claim 23, wherein the vaccine increases the vaccine efficacy of a standard of care influenza virus vaccine composition by an amount ranging from about 5% to about 100%, such as at least about 20%, or from about 40% to about 80%, such as from about 40% to about 60%.

34. The method according to claim 33, wherein the standard of care influenza virus vaccine composition is an inactivated influenza virus composition comprising inactivated influenza virus from an H1N1 strain, an H3N2 strain, a B/Victoria lineage, and a B/Yamagata lineage.

35. The method according to claim 33, wherein the standard of care influenza virus vaccine composition comprises recombinant influenza virus HA from an H1N1 strain, an H3N2 strain, a B/Victoria lineage, and a B/Yamagata lineage.

36. The method of any one of claims 24-35, comprising administering to the subject two doses of the vaccine with an interval of 2-6 weeks, optionally 4 weeks.


SEQUENCE LISTING (2 sequences)

SEQ ID NO: 1 - protein, 469 aa, Influenza A virus
MNPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTISEKSRLGYETFKVIEGWSNPKSKLQINRQVIVDRGNRSGYSGIFSVEGKSCINRCFYVELIRGRKEETEVLWTSNSIVVFCGTSGTYGTGSWPDGADINLMPI

SEQ ID NO: 2 - protein, 81 aa, Influenza A virus
MPMGSLQPLATLYLLGMLVASVLSAGSGIINETADDIVYRLTVIIDDRYESLKNLITLRADRLEMIINDNVSTILASIGSG